SEQUENCE LISTING (37 sequences)

SEQ ID NO: 1 - DNA, 823 bp, synthetic construct
  source 1..823: mol_type = other DNA
ttaattaaggcgcgccatgtctattgttacttttactcttactgatccttcttctgctcttattgctgaaattatgcaagctattgaaagacataacgtttctgttcttgaaggtcttagagatatttctaagcctactaagaagaagcaacaatctcaacctcaacaactttctagagcttctgctagacctcaacaacttcaacctggtccttctggttatcaagctaagaagcctgctaagcaaaaggctgaagttgttaagcctaagcaaaagcaacttgctcctcctattaataagaaggctgctaaggctaagctttatggtcttgaacaacattgtcctaagtatgctgaagctaagggtcttcaaaagcaaattggtatgacttattataagatttctgaaccttacgctcttcctgattttaaggttatggaagcttctgaagatcttgttgctgtttctgaaaaggatcctatgggttcttttgaaaagagactttattctatgggttttcctaagagacctattaagaacgttgttcctgtttttgaattttctgatcattatattgttgttttttttcctggttctaatgctgaaattgttaagaatgttcctaaggattctgtttctgattatgctgaagctcaacttgctgctcttcttgctgctagacaacaaattaatcaaattcatgaacttggtgatattcttcctactaattatcttaatgttcttgattctggtactcaagatgttgttgtttctgatgaagaagatgattctgattctgctcaataatagcccgggcctagg

SEQ ID NO: 2 - protein, 263 aa, synthetic construct
MSIVTFTLTDPSSALIAEIMQAIERHNVSVPEGLRDISKPTKKKQQSQPQQLSRASARPQQLQPGPSGYQAKKPAKQKAEVVKPKQKQLAPPINKKAAKAKLYGLEQHCPKYAEAKGLQKQIGMTYYKISEPYALPDFKVMEASEDLVAVSEKDPMGSFEKRLYSMGFPKRPIKNVVPVFEFSDHYIVVFFPGSNAEIVKNVPKDSVSDYAEAQLAALLAARQQINQIHELGDILPTNYLNVLDSGTQDVVVSDEEDDSDSAQ

SEQ ID NO: 3 - DNA, 56 bp, synthetic construct
  source 1..56: mol_type = other DNA
acaattaccaacaacaacaaacaacagacaacattacaattactatttacaattac

SEQ ID NO: 4 - DNA, 4851 bp, synthetic construct
  source 1..4851: mol_type = other DNA
atggcatacacacagacagctaccacatcagctttgctggacactgtccgaggaaacaactccttggtcaatgatctagcaaagcgtcgtctttacgacacagcggttgaagagtttaacgctcgtgaccgcaggcccaaggtgaacttttcaaaagtaataagcgaggagcagacgcttattgctacccgggcgtatccagaattccaaattacattttataacacgcaaaatgccgtgcattcgcttgcaggtggattgcgatctttagaactggaatatctgatgatgcaaattccctacggatcattgacttatgacataggcgggaattttgcatcgcatctgttcaagggacgagcatatgtacactgctgcatgcccaacctggacgttcgagacatcatgcggcacgaaggccagaaagacagtattgaactatacctttctaggctagagagaggggggaaaacagtccccaacttccaaaaggaagcatttgacagatacgcagaaattcctgaagacgctgtctgtcacaatactttccagacatgcgaacatcagccgatgcagcaatcaggcagagtgtatgccattgcgctacacagcatatatgacataccagccgatgagttcggggcggcactcttgaggaaaaatgtccatacgtgctatgccgctttccacttctccgagaacctgcttcttgaagattcatgcgtcaatttggacgaaatcaacgcgtgtttttcgcgcgatggagacaagttgaccttttcttttgcatcagagagtactcttaattactgtcatagttattctaatattcttaagtatgtgtgcaaaacttacttcccggcctctaatagagaggtttacatgaaggagtttttagtcaccagagttaatacctggttttgtaagttttctagaatagatacttttcttttgtacaaaggtgtggcccataaaagtgtagatagtgagcagttttatactgcaatggaagacgcatggcattacaaaaagactcttgcaatgtgcaacagcgagagaatcctccttgaggattcatcatcagtcaattactggtttcccaaaatgagggatatggtcatcgtaccattattcgacatttctttggagactagtaagaggacgcgcaaggaagtcttagtgtccaaggatttcgtgtttacagtgcttaaccacattcgaacataccaggcgaaagctcttacatacgcaaatgttttgtccttcgtcgaatcgattcgatcgagggtaatcattaacggtgtgacagcgaggtccgaatgggatgtggacaaatctttgttacaatccttgtccatgacgttttacctgcatactaagcttgccgttctaaaggatgacttactgattagcaagtttagtctcggttcgaaaacggtgtgccagcatgtgtgggatgagatttcgctggcgtttgggaacgcatttccctccgtgaaagagaggctcttgaacaggaaacttatcagagtggcaggcgacgcattagagatcagggtgcctgatctatatgtgaccttccacgacagattagtgactgagtacaaggcctctgtggacatgcctgcgcttgacattaggaagaagatggaagaaacggaagtgatgtacaatgcactttcagaattatcggtgttaagggagtctgacaaattcgatgttgatgttttttcccagatgtgccaatctttggaagttgacccaatgacggcagcgaaggttatagtcgcggtcatgagcaatgagagcggtctgactctcacatttgaacgacctactgaggcgaatgttgcgctagctttacaggatcaagagaaggcttcagaaggtgcattggtagttacctcaagagaagttgaagaaccgtccatgaagggttcgatggccagaggagagttacaattagctggtcttgctggagatcatccggaatcgtcctattctaagaacgaggagatagagtctttagagcagtttcatatggcgacggcagattcgttaattcgtaagcagatgagctcgattgtgtacacgggtccgattaaagttcagcaaatgaaaaactttatcgatagcctggtagcatcactatctgctgcggtgtcgaatctcgtcaagatcctcaaagatacagctgctattgaccttgaaacccgtcaaaagtttggagtcttggatgttgcatctaggaagtggttaatcaaaccaacggccaagagtcatgcatggggtgttgttgaaacccacgcgaggaagtatcatgtggcgcttttggaatatgatgagcagggtgtggtgacatgcgatgattggagaagagtagctgttagctctgagtctgttgtttattccgacatggcgaaactcagaactctgcgcagactgcttcgaaacggagaaccgcatgtcagtagcgcaaaggttgttcttgtggacggagttccgggctgtggaaaaaccaaagaaattctttccagggttaattttgatgaagatctaattttagtacctgggaagcaagccgcggaaatgatcagaagacgtgcgaattcctcagggattattgtggccacgaaggacaacgttaaaaccgttgattctttcatgatgaattttgggaaaagcacacgctgtcagttcaagaggttattcattgatgaagggttgatgttgcatactggttgtgttaattttcttgtggcgatgtcattgtgcgaaattgcatatgtttacggagacacacagcagattccatacatcaatagagtttcaggattcccgtaccccgcccattttgccaaattggaagttgacgaggtggagacacgcagaactactctccgttgtccagccgatgtcacacattatctgaacaggagatatgagggctttgtcatgagcacttcttcggttaaaaagtctgtttcgcaggagatggtcggcggagccgccgtgatcaatccgatctcaaaacccttgcatggcaagatcttgacttttacccaatcggataaagaagctctgctttcaagagggtattcagatgttcacactgtgcatgaagtgcaaggcgagacatactctgatgtttcactagttaggttaacccctacaccggtctccatcattgcaggagacagcccacatgttttggtcgcattgtcaaggcacacctgttcgctcaagtactacactgttgttatggatcctttagttagtatcattagagatctagagaaacttagctcgtacttgttagatatgtataaggtcgatgcaggaacacaatagcaattacagattgactcggtgttcaaaggttccaatctttttgttgcagcgccaaagactggtgatatttctgatatgcagttttactatgataagtgtctcccaggcaacagcaccatgatgaataattttgatgctgttaccatgaggttgactgacatttcattgaatgtcaaagattgcatattggatatgtctaagtctgttgctgcgcctaaggatcaaatcaaaccactaatacctatggtacgaacggcggcagaaatgccacgccagactggactattggaaaatttagtggcgatgattaaaagaaactttaacgcacccgagttgtctggcatcattgatattgaaaatactgcatctttggttgtagataagttttttgatagttatttgcttaaagaaaaaagaaaaccaaataaaaatgtttctttgttcagtagagagtctctcaatagatggttagaaaagcaggaacaggtaacaataggccagctcgcagattttgattttgtggatttgccagcagttgatcagtacagacacatgattaaagcacaacccaaacaaaagttggacacttcaatccaaacggagtacccggctttgcagacgattgtgtaccattcaaaaaagatcaatgcaatattcggcccgttgtttagtgagcttactaggcaattactggacagtgttgattcgagcagatttttgtttttcacaagaaagacaccagcgcagattgaggatttcttcggagatctcgacagtcatgtgccgatggatgtcttggagctggatatatcaaaatacgacaaatctcagaatgaattccactgtgcagtagaatacgagatctggcgaagattgggtttcgaagacttcttgggagaagtttggaaacaagggcatagaaagaccaccctcaaggattataccgcaggtataaaaacttgcatctggtatcaaagaaagagcggggacgtcacgacgttcattggaaacactgtgatcattgctgcatgtttggcctcgatgcttccgatggagaaaataatcaaaggagccttttgcggtgacgatagtctgctgtactttccaaagggttgtgagtttccggatgtgcaacactccgcgaatcttatgtggaattttgaagcaaaactgtttaaaaaacagtatggatacttttgcggaagatatgtaatacatcacgacagaggatgcattgtgtattacgatcccctaaagttgatctcgaaacttggtgctaaacacatcaaggattgggaacacttggaggagttcagaaggtctctttgtgatgttgctgtttcgttgaacaattgtgcgtattacacacagttggacgacgctgtatgggaggttcataagaccgcccctccaggttcgtttgtttataaaagtctggtgaagtatttgtctgataaagttctttttagaagtttgtttatagatggctctagttgttaa

SEQ ID NO: 5 - DNA, 807 bp, synthetic construct
  source 1..807: mol_type = other DNA
atggctctagttgttaaaggaaaagtgaatatcaatgagtttatcgacctgacaaaaatggagaagatcttaccgtcgatgtttacccctgtaaagagtgttatgtgttccaaagttgataaaataatggttcatgagaatgagtcattgtcaggggtgaaccttcttaaaggagttaagcttattgatagtggatacgtctgtttagccggtttggtcgtcacgggcgagtggaacttgcctgacaattgcagaggaggtgtgagcgtgtgtctggtggacaaaaggatggaaagagccgacgaggccactctcggatcttactacacagcagctgcaaagaaaagatttcagttcaaggtcgttcccaattatgctataaccacccaggacgcgatgaaaaacgtctggcaagttttagttaatattagaaatgtgaagatgtcagcgggtttctgtccgctttctctggagtttgtgtcggtgtgtattgtttatagaaataatataaaattaggtttgagagagaagattacaaacgtgagagacggagggcccatggaacttacagaagaagtcgttgatgagttcatggaagatgtccctatgtcgatcaggcttgcaaagtttcgatctcgaaccggaaaaaagagtgatgtccgcaaagggaaaaatagtagtagtgatcggtcagtgccgaacaagaactatagaaatgttaaggattttggaggaatgagttttaaaaagaataatttaatcgatgatgattcggaggctactgtcgccgaatcggattcgttttaa

SEQ ID NO: 6 - protein, 268 aa, synthetic construct
MALVVKGKVNINEFIDLTKMEKILPSMFTPVKSVMCSKVDKIMVHENESLSGVNLLKGVKLIDSGYVCLAGLVVTGEWNLPDNCRGGVSVCLVDKRMERADEATLGSYYTAAAKKRFQFKVVPNYAITTQDAMKNVWQVLVNIRNVKMSAGFCPLSLEFVSVCIVYRNNIKLGLREKITNVRDGGPMELTEEVVDEFMEDVPMSIRLAKFRSRTGKKSDVRKGKNSSSDRSVPNKNYRNVKDFGGMSFKKNNLIDDDSEATVAESDSF

SEQ ID NO: 7 - DNA, 792 bp, synthetic construct
  source 1..792: mol_type = other DNA
atgtctattgttacttttactcttactgatccttcttctgctcttattgctgaaattatgcaagctattgaaagacataacgtttctgttcctgaaggtcttagagatatttctaagcctactaagaagaagcaacaatctcaacctcaacaactttctagagcttctgctagacctcaacaacttcaacctggtccttctggttatcaagctaagaagcctgctaagcaaaaggctgaagttgttaagcctaagcaaaagcaacttgctcctcctattaataagaaggctgctaaggctaagctttatggtcttgaacaacattgtcctaagtatgctgaagctaagggtcttcaaaagcaaattggtatgacttattataagatttctgaaccttacgctcttcctgattttaaggttatggaagcttctgaagatcttgttgctgtttctgaaaaggatcctatgggttcttttgaaaagagactttattctatgggttttcctaagagacctattaagaacgttgttcctgtttttgaattttctgatcattatattgttgttttttttcctggttctaatgctgaaattgttaagaatgttcctaaggattctgtttctgattatgctgaagctcaacttgctgctcttcttgctgctagacaacaaattaatcaaattcatgaacttggtgatattcttcctactaattatcttaatgttcttgattctggtactcaagatgttgttgtttctgatgaagaagatgattctgattctgctcaataa

SEQ ID NO: 8 - protein, 263 aa, synthetic construct; identical to SEQ ID NO: 2

SEQ ID NO: 9 - DNA, 823 bp, synthetic construct
  source 1..823: mol_type = other DNA
aattaattccgcgcggtacagataacaatgaaaatgagaatgactaggaagaagacgagaataacgactttaatacgttcgataactttctgtattgcaaagacaaggacttccagaatctctataaagattcggatgattcttcttcgttgttagagttggagttgttgaaagatctcgaagacgatctggagttgttgaagttggaccaggaagaccaatagttcgattcttcggacgattcgttttccgacttcaacaattcggattcgttttcgttgaacgaggaggataattattcttccgacgattccgattcgaaataccagaacttgttgtaacaggattcatacgacttcgattcccagaagttttcgtttaaccatactgaataatattctaaagacttggaatgcgagaaggactaaaattccaataccttcgaagacttctagaacaacgacaaagacttttcctaggatacccaagaaaacttttctctgaaataagatacccaaaaggattctctggataattattgcaacaaggacaaaaacttaaaagactagtaatataacaacaaaaaaaaggaccaagattacgactttaacaattcttacaaggattcctaagacaaagactaatacgacttcgagttgaacgacgagaagaacgacgatctgttgtttaattagtttaagtacttgaaccactataagaaggatgattaatagaattacaagaactaagaccatgagttctacaacaacaaagactacttcttctactaagactaagacgagttattatcgggcccggatcc

SEQ ID NO: 10 - DNA, 56 bp, synthetic construct
  source 1..56: mol_type = other DNA
gtaattgtaaatagtaattgtaatgttgtctgttgtttgttgttgttggtaattgt

SEQ ID NO: 11 - DNA, 4851 bp, synthetic construct
  source 1..4851: mol_type = other DNA
aattgttgatctcggtagatatttgtttgaagatttttcttgaaatagtctgtttatgaagtggtctgaaaatatttgtttgcttggacctccccgccagaatacttggagggtatgtcgcagcaggttgacacacattatgcgtgttaacaagttgctttgtcgttgtagtgtttctctggaagacttgaggaggttcacaagggttaggaactacacaaatcgtggttcaaagctctagttgaaatcccctagcattatgtgttacgtaggagacagcactacataatgtatagaaggcgttttcataggtatgacaaaaaatttgtcaaaacgaagttttaaggtgtattctaagcgcctcacaacgtgtaggcctttgagtgttgggaaacctttcatgtcgtctgatagcagtggcgttttccgaggaaactaataaaagaggtagccttcgtagctccggtttgtacgtcgttactagtgtcacaaaggttacttgcagcactgcaggggcgagaaagaaactatggtctacgttcaaaaatatggacgccatattaggaactcccaccagaaagatacgggaacaaaggtttgaagagggttcttcagaagctttgggttagaagcggtctagagcataagatgacgtgtcaccttaagtaagactctaaacagcataaaactatataggtcgaggttctgtaggtagccgtgtactgacagctctagaggcttctttaggagttagacgcgaccacagaaagaacactttttgtttttagacgagcttagttgtgacaggtcattaacggatcattcgagtgatttgttgcccggcttataacgtaactagaaaaaacttaccatgtgttagcagacgtttcggcccatgaggcaaacctaacttcacaggttgaaaacaaacccaacacgaaattagtacacagacatgactagttgacgaccgtttaggtgttttagttttagacgctcgaccggataacaatggacaaggacgaaaagattggtagataactctctgagagatgacttgtttctttgtaaaaataaaccaaaagaaaaaagaaattcgtttattgatagttttttgaatagatgttggtttctacgtcataaaagttatagttactacggtctgttgagcccacgcaatttcaaagaaaattagtagcggtgatttaaaaggttatcaggtcagaccgcaccgtaaagacggcggcaagcatggtatccataatcaccaaactaaactaggaatccgcgtcgttgtctgaatctgtataggttatacgttagaaactgtaagttactttacagtcagttggagtaccattgtcgtagttttaataagtagtaccacgacaacggaccctctgtgaatagtatcattttgacgtatagtctttatagtggtcagaaaccgcgacgttgtttttctaaccttggaaacttgtggctcagttagacattaacgataacacaaggacgtagctggaatatgtatagattgttcatgctcgattcaaagagatctagagattactatgattgatttcctaggtattgttgtcacatcatgaactcgcttgtccacacggaactgttacgctggttttgtacacccgacagaggacgttactacctctggccacatccccaattggattgatcactttgtagtctcatacagagcggaacgtgaagtacgtgtcacacttgtagacttatgggagaactttcgtctcgaagaaataggctaacccattttcagttctagaacggtacgttcccaaaactctagcctaactagtgccgccgaggcggctggtagaggacgctttgtctgaaaaattggcttcttcacgagtactgtttcgggagtatagaggacaagtctattacacactgtagccgacctgttgcctctcatcaagacgcacagaggtggagcagttgaaggttaaaccgttttacccgccccatgcccttaggactttgagataactacataccttagacgacacacagaggcatttgtatacgttaaagcgtgttactgtagcggtgttcttttaattgtgttggtcatacgttgtagttgggaagtagttacttattggagaacttgactgtcgcacacgaaaagggttttaagtagtactttcttagttgccaaaattgcaacaggaagcaccggtgttattagggactccttaagcgtgcagaagactagtaaaggcgccgaacgaagggtccatgattttaatctagaagtagttttaattgggacctttcttaaagaaaccaaaaaggtgtcgggccttgaggcaggtgttcttgttggaaacgcgatgactgtacgccaagaggcaaagcttcgtcagacgcgtctcaagactcaaagcggtacagccttatttgttgtctgagtctcgattgtcgatgagaagaggttagtagcgtacagtggtgtgggacgagtagtataaggttttcgcggtgtactatgaaggagcgcacccaaagttgttgtggggtacgtactgagaaccggcaaccaaactaattggtgaaggatctacgttgtaggttctgaggtttgaaaactgcccaaagttccagttatcgtcgacatagaaactcctagaactgctctaagctgtggcgtcgtctatcactacgatggtccgatagctatttcaaaaagtaaacgacttgaaattagcctgggcacatgtgttagctcgagtagacgaatgcttaattgcttagacggcagcggtatactttgacgagatttctgagatagaggagcaagaatcttatcctgctaaggcctactagaggtcgttctggtcgattaacattgagaggagaccggtagcttgggaagtacctgccaagaagttgaagagaactccattgatggttacgtggaagacttcggaagagaactaggacatttcgatcgcgttgtaagcggagtcatccagcaagtttacactctcagtctggcgagagtaacgagtactggcgctgatattggaagcgacggcagtaacccagttgaaggtttctaaccgtgtagacccttttttgtagttgtagcttaaacagtctgagggaattgtggctattaagactttcacgtaacatgtagtgaaggcaaagaaggtagaagaaggattacagttcgcgtccgtacaggtgtctccggaacatgagtcagtgattagacagcaccttccagtgtatatctagtccgtgggactagagattacgcagcggacggtgagactattcaaaggacaagttctcggagagaaagtgcctccctttacgcaagggtttgcggtcgctttagagtagggtgtgtacgaccgtgtggcaaaagcttggctctgatttgaacgattagtcattcagtaggaaatcttgccgttcgaatcatacgtccattttgcagtacctgttcctaacattgtttctaaacaggtgtagggtaagcctggagcgacagtgtggcaattactaatgggagctagcttagctaagctgcttcctgttttgtaaacgcatacattctcgaaagcggaccatacaagcttacaccaattcgtgacatttgtgctttaggaacctgtgattctgaaggaacgcgcaggagaatgatcagaggtttctttacagcttattaccatgctactggtatagggagtaaaaccctttggtcattaactgactactacttaggagttcctcctaagagagcgacaacgtgtaacgttctcagaaaaacattacggtacgcagaaggtaacgtcatattttgacgagtgatagatgtgaaaatacccggtgtggaaacatgttttcttttcatagataagatcttttgaatgttttggtccataattgagaccactgatttttgaggaagtacatttggagagataatctccggcccttcattcaaaacgtgtgtatgaattcttataatcttattgatactgtcattaattctcatgagagactacgttttcttttccagttgaacagaggtagcgcgctttttgtgcgcaactaaagcaggtttaactgcgtacttagaagttcttcgtccaagagcctcttcacctttcgccgtatcgtgcatacctgtaaaaaggagttctcacggcggggcttgagtagccgaccatacagtatatacgacacatcgcgttaccgtatgtgagacggactaacgacgtagccgactacaagcgtacagacctttcataacactgtctgtcgcagaagtccttaaagacgcatagacagtttacgaaggaaaaccttcaacccctgacaaaaggggggagagagatcggatctttccatatcaagttatgacagaaagaccggaagcacggcgtactacagagcttgcaggtccaacccgtacgtcgtcacatgtatacgagcagggaacttgtctacgctacgttttaagggcggatacagtattcagttactaggcatcccttaaacgtagtagtctataaggtcaagatttctagcgttaggtggacgttcgcttacgtgccgtaaaacgcacaatattttacattaaaccttaagacctatgcgggcccatcgttattcgcagacgaggagcgaataatgaaaacttttcaagtggaacccggacgccagtgctcgcaatttgagaagttggcgacacagcatttctgctgcgaaacgatctagtaactggttcctcaacaaaggagcctgtcacaggtcgtttcgactacaccatcgacagacacacatacggta

SEQ ID NO: 12 - DNA, 807 bp, synthetic construct
  source 1..807: mol_type = other DNA
ttaaaacgaatccgattcggcgacagtagcctccgaatcatcatcgattaaattattctttttaaaactcattcctccaaaatccttaacatttctatagttcttgttcggcactgaccgatcactactactatttttccctttgcggacatcactcttttttccggttcgagatcgaaactttgcaagcctgatcgacatagggacatcttccatgaactcatcaacgacttcttctgtaagttccatgggccctccgtctctcacgtttgtaatcttctctctcaaacctaattttatattatttctataaacaatacacaccgacacaaactccagagaaagcggacagaaacccgctgacatcttcacatttctaatattaactaaaacttgccagacgtttttcatcgcgtcctgggtggttatagcataattgggaacgaccttgaactgaaatcttttctttgcagctgctgtgtagtaagatccgagagtggcctcgtcggctctttccatccttttgtccaccagacacacgctcacacctcctctgcaattgtcaggcaagttccactcgcccgtgacgaccaaaccggctaaacagacgtatccactatcaataagcttaactcctttaagaaggttcacccctgacaatgactcattctcatgaaccattattttatcaactttggaacacataacactctttacaggggtaaacatcgacggtaagatcttctccatttttgtcaggtcgataaactcattgatattcacttttcctttaacaactagagccat

SEQ ID NO: 13 - DNA, 792 bp, synthetic construct
  source 1..792: mol_type = other DNA
ttattgagcagaatcagaatcatcttcttcatcagaaacaacaacatcttgagtaccagaatcaagaacattaagataattagtaggaagaatatcaccaagttcatgaatttgattaatttgttgtctagcagcaagaagagcagcaagttgagcttcagcataatcagaaacagaatccttaggaacattcttaacaatttcagcattagaaccaggaaaaaaaacaacaatataatgatcagaaaattcaaaaacaggaacaacgttcttaataggtctcttaggaaaacccatagaataaagtctcttttcaaaagaacccataggatccttttcagaaacagcaacaagatcttcagaagcttccataaccttaaaatcaggaagagcgtaaggttcagaaatcttataataagtcataccaatttgcttttgaagacccttagcttcagcatacttaggacaatgttgttcaagaccataaagcttagccttagcagccttcttattaataggaggagcaagttgcttttgcttaggcttaacaacttcagccttttgcttagcaggcttcttagcttgataaccagaaggaccaggttgaagttgttgaggtctagcagaagctctagaaagttgttgaggttgagattgttgcttcttcttagtaggcttagaaatatctctaagaccttcaggaacagaaacgttatgtctttcaatagcttgcataatttcagcaataagagcagaagaaggatcagtaagagtaaaagtaacaatagacat

SEQ ID NO: 14 - DNA, 1149 bp, synthetic construct
  source 1..1149: mol_type = other DNA
ctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtggagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctgggccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcat

SEQ ID NO: 15 - DNA, 1149 bp, synthetic construct
  source 1..1149: mol_type = other DNA
atgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggcccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggctccacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctag

SEQ ID NO: 16 - protein, 382 aa, synthetic construct
MNRTFDRKAYRQELIDAGFSAEDAETIASRTVMRAPRETFQSVGSMAQQATAKIERDSVQLAPPALPAPSAAVERSRRLEQEAAGLAKSMTIDTRGTMTTKKRKTAGEDLAKQVSEAKQAALLKHTKQQIKEMQLSLFDIAPWPDTMRAMPNDTARSALFTTRNKKIPREALQNKVIFHVNKDVKITYTGVELRADDDELVWQQVLEYAKRTPIGEPITFTFYELCQDLGWSINGRYYTKAEECLSRLQATAMGFTSDRVGHLESVSLLHRFRVLDRGKKTSRCQVLIDEEIVVLFAGDHYTKFIWEKYRKLSPTARRMFDYFSSHREPYPLKLETFRLMCGSDSTRVKKWREQVGEACEELRGSGLVEHAWVNDDLVHCKR

SEQ ID NO: 17 - DNA, 795 bp, synthetic construct
  source 1..795: mol_type = other DNA
ctaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccat

SEQ ID NO: 18 - DNA, 795 bp, synthetic construct
  source 1..795: mol_type = other DNA
atggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttag

SEQ ID NO: 19 - protein, 265 aa, synthetic construct
FLEDLLIYYKIKEWDPKIGLLDFFLEVYQEEGIDERISRVCFAIDYWKDARGSRGLDIFGSVKGDKVFINSDGLDGSFVLEEEPKETKLFDYLRPDKFPTDEEWNECYCDVDALDNNLLYDLEALRSDLSNTYPCYCDSIDISHFLRICEAYLEIKEPSQEDEYEESCLVGDAESMLLNSWGDHREFHLVKPVPLKGELWLMMDKEREVDYTTGKYRSDTMKLYLNENEGVLKYVAKPSMGETDKVCRYKEILKKLEPSIRMKAM

SEQ ID NO: 20 - DNA, 532 bp, synthetic construct
  source 1..532: mol_type = other DNA
agcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggcc

SEQ ID NO: 21 - DNA, 532 bp, synthetic construct
  source 1..532: mol_type = other DNA
ggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgct

SEQ ID NO: 22 - DNA, 615 bp, synthetic construct
  source 1..615: mol_type = other DNA
gggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccggccgcggtgtctcgcacacggcttcgacggcgtttctggcgcgtttgcagggccatagacggccgccagcccagcggcgagggcaaccag

SEQ ID NO: 23 - DNA, 615 bp, synthetic construct
  source 1..615: mol_type = other DNA
ctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctccc

SEQ ID NO: 24 - DNA, 26 bp, synthetic construct
  source 1..26: mol_type = other DNA
gtggcaggatatattgtggtgtaaac

SEQ ID NO: 25 - DNA, 27 bp, synthetic construct
  source 1..27: mol_type = other DNA
gtttacaccacaatatatcctgccacc

SEQ ID NO: 26 - DNA, 17 bp, synthetic construct
  source 1..17: mol_type = other DNA
cgaggtcgacggtatcg

SEQ ID NO: 27 - DNA, 17 bp, synthetic construct
  source 1..17: mol_type = other DNA
cgataccgtcgacctcg

SEQ ID NO: 28 - DNA, 45 bp, synthetic construct
  source 1..45: mol_type = other DNA
gcaagaccttcctctatataaggaagttcatttcatttggagagg

SEQ ID NO: 29 - DNA, 45 bp, synthetic construct
  source 1..45: mol_type = other DNA
cctctccaaatgaaatgaacttccttatatagaggaaggtcttgc

SEQ ID NO: 30 - DNA, 11414 bp, synthetic construct
  source 1..11414: mol_type = other DNA
gtatttttacaacaattaccaacaacaacaaacaacagacaacattacaattactatttacaattacaatggcatacacacagacagctaccacatcagctttgctggacactgtccgaggaaacaactccttggtcaatgatctagcaaagcgtcgtctttacgacacagcggttgaagagtttaacgctcgtgaccgcaggcccaaggtgaacttttcaaaagtaataagcgaggagcagacgcttattgctacccgggcgtatccagaattccaaattacattttataacacgcaaaatgccgtgcattcgcttgcaggtggattgcgatctttagaactggaatatctgatgatgcaaattccctacggatcattgacttatgacataggcgggaattttgcatcgcatctgttcaagggacgagcatatgtacactgctgcatgcccaacctggacgttcgagacatcatgcggcacgaaggccagaaagacagtattgaactatacctttctaggctagagagaggggggaaaacagtccccaacttccaaaaggaagcatttgacagatacgcagaaattcctgaagacgctgtctgtcacaatactttccagacatgcgaacatcagccgatgcagcaatcaggcagagtgtatgccattgcgctacacagcatatatgacataccagccgatgagttcggggcggcactcttgaggaaaaatgtccatacgtgctatgccgctttccacttctccgagaacctgcttcttgaagattcatgcgtcaatttggacgaaatcaacgcgtgtttttcgcgcgatggagacaagttgaccttttcttttgcatcagagagtactcttaattactgtcatagttattctaatattcttaagtatgtgtgcaaaacttacttcccggcctctaatagagaggtttacatgaaggagtttttagtcaccagagttaatacctggttttgtaagttttctagaatagatacttttcttttgtacaaaggtgtggcccataaaagtgtagatagtgagcagttttatactgcaatggaagacgcatggcattacaaaaagactcttgcaatgtgcaacagcgagagaatcctccttgaggattcatcatcagtcaattactggtttcccaaaatgagggatatggtcatcgtaccattattcgacatttctttggagactagtaagaggacgcgcaaggaagtcttagtgtccaaggatttcgtgtttacagtgcttaaccacattcgaacataccaggcgaaagctcttacatacgcaaatgttttgtccttcgtcgaatcgattcgatcgagggtaatcattaacggtgtgacagcgaggtccgaatgggatgtggacaaatctttgttacaatccttgtccatgacgttttacctgcatactaagcttgccgttctaaaggatgacttactgattagcaagtttagtctcggttcgaaaacggtgtgccagcatgtgtgggatgagatttcgctggcgtttgggaacgcatttccctccgtgaaagagaggctcttgaacaggaaacttatcagagtggcaggcgacgcattagagatcagggtgcctgatctatatgtgaccttccacgacagattagtgactgagtacaaggcctctgtggacatgcctgcgcttgacattaggaagaagatggaagaaacggaagtgatgtacaatgcactttcagaattatcggtgttaagggagtctgacaaattcgatgttgatgttttttcccagatgtgccaatctttggaagttgacccaatgacggcagcgaaggttatagtcgcggtcatgagcaatgagagcggtctgactctcacatttgaacgacctactgaggcgaatgttgcgctagctttacaggatcaagagaaggcttcagaaggtgcattggtagttacctcaagagaagttgaagaaccgtccatgaagggttcgatggccagaggagagttacaattagctggtcttgctggagatcatccggaatcgtcctattctaagaacgaggagatagagtctttagagcagtttcatatggcgacggcagattcgttaattcgtaagcagatgagctcgattgtgtacacgggtccgattaaagttcagcaaatgaaaaactttatcgatagcctggtagcatcactatctgctgcggtgtcgaatctcgtcaagatcctcaaagatacagctgctattgaccttgaaacccgtcaaaagtttggagtcttggatgttgcatctaggaagtggttaatcaaaccaacggccaagagtcatgcatggggtgttgttgaaacccacgcgaggaagtatcatgtggcgcttttggaatatgatgagcagggtgtggtgacatgcgatgattggagaagagtagctgttagctctgagtctgttgtttattccgacatggcgaaactcagaactctgcgcagactgcttcgaaacggagaaccgcatgtcagtagcgcaaaggttgttcttgtggacggagttccgggctgtggaaaaaccaaagaaattctttccagggttaattttgatgaagatctaattttagtacctgggaagcaagccgcggaaatgatcagaagacgtgcgaattcctcagggattattgtggccacgaaggacaacgttaaaaccgttgattctttcatgatgaattttgggaaaagcacacgctgtcagttcaagaggttattcattgatgaagggttgatgttgcatactggttgtgttaattttcttgtggcgatgtcattgtgcgaaattgcatatgtttacggagacacacagcagattccatacatcaatagagtttcaggattcccgtaccccgcccattttgccaaattggaagttgacgaggtggagacacgcagaactactctccgttgtccagccgatgtcacacattatctgaacaggagatatgagggctttgtcatgagcacttcttcggttaaaaagtctgtttcgcaggagatggtcggcggagccgccgtgatcaatccgatctcaaaacccttgcatggcaagatcttgacttttacccaatcggataaagaagctctgctttcaagagggtattcagatgttcacactgtgcatgaagtgcaaggcgagacatactctgatgtttcactagttaggttaacccctacaccggtctccatcattgcaggagacagcccacatgttttggtcgcattgtcaaggcacacctgttcgctcaagtactacactgttgttatggatcctttagttagtatcattagagatctagagaaacttagctcgtacttgttagatatgtataaggtcgatgcaggaacacaatagcaattacagattgactcggtgttcaaaggttccaatctttttgttgcagcgccaaagactggtgatatttctgatatgcagttttactatgataagtgtctcccaggcaacagcaccatgatgaataattttgatgctgttaccatgaggttgactgacatttcattgaatgtcaaagattgcatattggatatgtctaagtctgttgctgcgcctaaggatcaaatcaaaccactaatacctatggtacgaacggcggcagaaatgccacgccagactggactattggaaaatttagtggcgatgattaaaagaaactttaacgcacccgagttgtctggcatcattgatattgaaaatactgcatctttggttgtagataagttttttgatagttatttgcttaaagaaaaaagaaaaccaaataaaaatgtttctttgttcagtagagagtctctcaatagatggttagaaaagcaggaacaggtaacaataggccagctcgcagattttgattttgtggatttgccagcagttgatcagtacagacacatgattaaagcacaacccaaacaaaagttggacacttcaatccaaacggagtacccggctttgcagacgattgtgtaccattcaaaaaagatcaatgcaatattcggcccgttgtttagtgagcttactaggcaattactggacagtgttgattcgagcagatttttgtttttcacaagaaagacaccagcgcagattgaggatttcttcggagatctcgacagtcatgtgccgatggatgtcttggagctggatatatcaaaatacgacaaatctcagaatgaattccactgtgcagtagaatacgagatctggcgaagattgggtttcgaagacttcttgggagaagtttggaaacaagggcatagaaagaccaccctcaaggattataccgcaggtataaaaacttgcatctggtatcaaagaaagagcggggacgtcacgacgttcattggaaacactgtgatcattgctgcatgtttggcctcgatgcttccgatggagaaaataatcaaaggagccttttgcggtgacgatagtctgctgtactttccaaagggttgtgagtttccggatgtgcaacactccgcgaatcttatgtggaattttgaagcaaaactgtttaaaaaacagtatggatacttttgcggaagatatgtaatacatcacgacagaggatgcattgtgtattacgatcccctaaagttgatctcgaaacttggtgctaaacacatcaaggattgggaacacttggaggagttcagaaggtctctttgtgatgttgctgtttcgttgaacaattgtgcgtattacacacagttggacgacgctgtatgggaggttcataagaccgcccctccaggttcgtttgtttataaaagtctggtgaagtatttgtctgataaagttctttttagaagtttgtttatagatggctctagttgttaaaggaaaagtgaatatcaatgagtttatcgacctgacaaaaatggagaagatcttaccgtcgatgtttacccctgtaaagagtgttatgtgttccaaagttgataaaataatggttcatgagaatgagtcattgtcaggggtgaaccttcttaaaggagttaagcttattgatagtggatacgtctgtttagccggtttggtcgtcacgggcgagtggaacttgcctgacaattgcagaggaggtgtgagcgtgtgtctggtggacaaaaggatggaaagagccgacgaggccactctcggatcttactacacagcagctgcaaagaaaagatttcagttcaaggtcgttcccaattatgctataaccacccaggacgcgatgaaaaacgtctggcaagttttagttaatattagaaatgtgaagatgtcagcgggtttctgtccgctttctctggagtttgtgtcggtgtgtattgtttatagaaataatataaaattaggtttgagagagaagattacaaacgtgagagacggagggcccatggaacttacagaagaagtcgttgatgagttcatggaagatgtccctatgtcgatcaggcttgcaaagtttcgatctcgaaccggaaaaaagagtgatgtccgcaaagggaaaaatagtagtagtgatcggtcagtgccgaacaagaactatagaaatgttaaggattttggaggaatgagttttaaaaagaataatttaatcgatgatgattcggaggctactgtcgccgaatcggattcgttttaaatagatcttacagtatcactactccatctcagttcgtgttcttgtcattaattaaggcgcgccatgtctattgttacttttactcttactgatccttcttctgctcttattgctgaaattatgcaagctattgaaagacataacgtttctgttcctgaaggtcttagagatatttctaagcctactaagaagaagcaacaatctcaacctcaacaactttctagagcttctgctagacctcaacaacttcaacctggtccttctggttatcaagctaagaagcctgctaagcaaaaggctgaagttgttaagcctaagcaaaagcaacttgctcctcctattaataagaaggctgctaaggctaagctttatggtcttgaacaacattgtcctaagtatgctgaagctaagggtcttcaaaagcaaattggtatgacttattataagatttctgaaccttacgctcttcctgattttaaggttatggaagcttctgaagatcttgttgctgtttctgaaaaggatcctatgggttcttttgaaaagagactttattctatgggttttcctaagagacctattaagaacgttgttcctgtttttgaattttctgatcattatattgttgttttttttcctggttctaatgctgaaattgttaagaatgttcctaaggattctgtttctgattatgctgaagctcaacttgctgctcttcttgctgctagacaacaaattaatcaaattcatgaacttggtgatattcttcctactaattatcttaatgttcttgattctggtactcaagatgttgttgtttctgatgaagaagatgattctgattctgctcaataatagcccgggcctagggcggccgcggtcctgcaacttgaggtagtcaagatgcataataaataacggattgtgtccgtaatcacacgtggtgcgtacgataacgcatagtgtttttccctccacttaaatcgaagggttgtgtcttggatcgcgcgggtcaaatgtatatggttcatatacatccgcaggcacgtaataaagcgaggggttcgaatccccccgttacccccggtaggggcccaggtacccggatgtgttttccgggttgatgagtccgtgaggacgaaacctggctgcaggcatgcaagcttggcgtaatcatggtcatagcctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacctgcagcccggccgggggatccactagcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacaggagatctcagtaaagcgctggctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtggagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctgggccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccacatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccacaattatgggtgatgctgccaactcgagagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccggccgcggtgtctcgcacacggcttcgacggcgtttctggcgcgtttgcagggccatagacggccgccagcccagcggcgagggcaaccagcccggtgagctctagtggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggttttggtaccgggccccccctcgaggtcgacggtatcgataagcttgatatcgaattcctgcaggtcaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagagg

SEQ ID NO: 31 - DNA, 11414 bp, synthetic construct
  source 1..11414: mol_type = other DNA
cctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttgacctgcaggaattcgatatcaagcttatcgataccgtcgacctcgagggggggcccggtaccaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccactagagctcaccgggctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctctcgagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggcccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggctccacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagccagcgctttactgagatctcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgctagtggatcccccggccgggctgcaggtcactggattttggttttaggaattagaaattttattgatagaagtattttacaaatacaaatacatactaagggtttcttatatgctcaacacatgagcgaaaccctataagaaccctaattcccttatctgggaactactcacacattattctggagaaaaatagagagagatagatttgtagagagagactggtgatttttgcggactctaggctatgaccatgattacgccaagcttgcatgcctgcagccaggtttcgtcctcacggactcatcaacccggaaaacacatccgggtacctgggcccctaccgggggtaacggggggattcgaacccctcgctttattacgtgcctgcggatgtatatgaaccatatacatttgacccgcgcgatccaagacacaacccttcgatttaagtggagggaaaaacactatgcgttatcgtacgcaccacgtgtgattacggacacaatccgttatttattatgcatcttgactacctcaagttgcaggaccgcggccgccctaggcccgggctattattgagcagaatcagaatcatcttcttcatcagaaacaacaacatcttgagtaccagaatcaagaacattaagataattagtaggaagaatatcaccaagttcatgaatttgattaatttgttgtctagcagcaagaagagcagcaagttgagcttcagcataatcagaaacagaatccttaggaacattcttaacaatttcagcattagaaccaggaaaaaaaacaacaatataatgatcagaaaattcaaaaacaggaacaacgttcttaataggtctcttaggaaaacccatagaataaagtctcttttcaaaagaacccataggatccttttcagaaacagcaacaagatcttcagaagcttccataaccttaaaatcaggaagagcgtaaggttcagaaatcttataataagtcataccaatttgcttttgaagacccttagcttcagcatacttaggacaatgttgttcaagaccataaagcttagccttagcagccttcttattaataggaggagcaagttgcttttgcttaggcttaacaacttcagccttttgcttagcaggcttcttagcttgataaccagaaggaccaggttgaagttgttgaggtctagcagaagctctagaaagttgttgaggttgagattgttgcttcttcttagtaggcttagaaatatctctaagaccttcaggaacagaaacgttatgtctttcaatagcttgcataatttcagcaataagagcagaagaaggatcagtaagagtaaaagtaacaatagacatggcgcgccttaattaatgacaagaacacgaactgagatggagtagtgatactgtaagatctatttaaaacgaatccgattcggcgacagtagcctccgaatcatcatcgattaaattattctttttaaaactcattcctccaaaatccttaacatttctatagttcttgttcggcactgaccgatcactactactatttttccctttgcggacatcactcttttttccggttcgagatcgaaactttgcaagcctgatcgacatagggacatcttccatgaactcatcaacgacttcttctgtaagttccatgggccctccgtctctcacgtttgtaatcttctctctcaaacctaattttatattatttctataaacaatacacaccgacacaaactccagagaaagcggacagaaacccgctgacatcttcacatttctaatattaactaaaacttgccagacgtttttcatcgcgtcctgggtggttatagcataattgggaacgaccttgaactgaaatcttttctttgcagctgctgtgtagtaagatccgagagtggcctcgtcggctctttccatccttttgtccaccagacacacgctcacacctcctctgcaattgtcaggcaagttccactcgcccgtgacgaccaaaccggctaaacagacgtatccactatcaataagcttaactcctttaagaaggttcacccctgacaatgactcattctcatgaaccattattttatcaactttggaacacataacactctttacaggggtaaacatcgacggtaagatcttctccatttttgtcaggtcgataaactcattgatattcacttttcctttaacaactagagccatctataaacaaacttctaaaaagaactttatcagacaaatacttcaccagacttttataaacaaacgaacctggaggggcggtcttatgaacctcccatacagcgtcgtccaactgtgtgtaatacgcacaattgttcaacgaaacagcaacatcacaaagagaccttctgaactcctccaagtgttcccaatccttgatgtgtttagcaccaagtttcgagatcaactttaggggatcgtaatacacaatgcatcctctgtcgtgatgtattacatatcttccgcaaaagtatccatactgttttttaaacagttttgcttcaaaattccacataagattcgcggagtgttgcacatccggaaactcacaaccctttggaaagtacagcagactatcgtcaccgcaaaaggctcctttgattattttctccatcggaagcatcgaggccaaacatgcagcaatgatcacagtgtttccaatgaacgtcgtgacgtccccgctctttctttgataccagatgcaagtttttatacctgcggtataatccttgagggtggtctttctatgcccttgtttccaaacttctcccaagaagtcttcgaaacccaatcttcgccagatctcgtattctactgcacagtggaattcattctgagatttgtcgtattttgatatatccagctccaagacatccatcggcacatgactgtcgagatctccgaagaaatcctcaatctgcgctggtgtctttcttgtgaaaaacaaaaatctgctcgaatcaacactgtccagtaattgcctagtaagctcactaaacaacgggccgaatattgcattgatcttttttgaatggtacacaatcgtctgcaaagccgggtactccgtttggattgaagtgtccaacttttgtttgggttgtgctttaatcatgtgtctgtactgatcaactgctggcaaatccacaaaatcaaaatctgcgagctggcctattgttacctgttcctgcttttctaaccatctattgagagactctctactgaacaaagaaacatttttatttggttttcttttttctttaagcaaataactatcaaaaaacttatctacaaccaaagatgcagtattttcaatatcaatgatgccagacaactcgggtgcgttaaagtttcttttaatcatcgccactaaattttccaatagtccagtctggcgtggcatttctgccgccgttcgtaccataggtattagtggtttgatttgatccttaggcgcagcaacagacttagacatatccaatatgcaatctttgacattcaatgaaatgtcagtcaacctcatggtaacagcatcaaaattattcatcatggtgctgttgcctgggagacacttatcatagtaaaactgcatatcagaaatatcaccagtctttggcgctgcaacaaaaagattggaacctttgaacaccgagtcaatctgtaattgctattgtgttcctgcatcgaccttatacatatctaacaagtacgagctaagtttctctagatctctaatgatactaactaaaggatccataacaacagtgtagtacttgagcgaacaggtgtgccttgacaatgcgaccaaaacatgtgggctgtctcctgcaatgatggagaccggtgtaggggttaacctaactagtgaaacatcagagtatgtctcgccttgcacttcatgcacagtgtgaacatctgaataccctcttgaaagcagagcttctttatccgattgggtaaaagtcaagatcttgccatgcaagggttttgagatcggattgatcacggcggctccgccgaccatctcctgcgaaacagactttttaaccgaagaagtgctcatgacaaagccctcatatctcctgttcagataatgtgtgacatcggctggacaacggagagtagttctgcgtgtctccacctcgtcaacttccaatttggcaaaatgggcggggtacgggaatcctgaaactctattgatgtatggaatctgctgtgtgtctccgtaaacatatgcaatttcgcacaatgacatcgccacaagaaaattaacacaaccagtatgcaacatcaacccttcatcaatgaataacctcttgaactgacagcgtgtgcttttcccaaaattcatcatgaaagaatcaacggttttaacgttgtccttcgtggccacaataatccctgaggaattcgcacgtcttctgatcatttccgcggcttgcttcccaggtactaaaattagatcttcatcaaaattaaccctggaaagaatttctttggtttttccacagcccggaactccgtccacaagaacaacctttgcgctactgacatgcggttctccgtttcgaagcagtctgcgcagagttctgagtttcgccatgtcggaataaacaacagactcagagctaacagctactcttctccaatcatcgcatgtcaccacaccctgctcatcatattccaaaagcgccacatgatacttcctcgcgtgggtttcaacaacaccccatgcatgactcttggccgttggtttgattaaccacttcctagatgcaacatccaagactccaaacttttgacgggtttcaaggtcaatagcagctgtatctttgaggatcttgacgagattcgacaccgcagcagatagtgatgctaccaggctatcgataaagtttttcatttgctgaactttaatcggacccgtgtacacaatcgagctcatctgcttacgaattaacgaatctgccgtcgccatatgaaactgctctaaagactctatctcctcgttcttagaataggacgattccggatgatctccagcaagaccagctaattgtaactctcctctggccatcgaacccttcatggacggttcttcaacttctcttgaggtaactaccaatgcaccttctgaagccttctcttgatcctgtaaagctagcgcaacattcgcctcagtaggtcgttcaaatgtgagagtcagaccgctctcattgctcatgaccgcgactataaccttcgctgccgtcattgggtcaacttccaaagattggcacatctgggaaaaaacatcaacatcgaatttgtcagactcccttaacaccgataattctgaaagtgcattgtacatcacttccgtttcttccatcttcttcctaatgtcaagcgcaggcatgtccacagaggccttgtactcagtcactaatctgtcgtggaaggtcacatatagatcaggcaccctgatctctaatgcgtcgcctgccactctgataagtttcctgttcaagagcctctctttcacggagggaaatgcgttcccaaacgccagcgaaatctcatcccacacatgctggcacaccgttttcgaaccgagactaaacttgctaatcagtaagtcatcctttagaacggcaagcttagtatgcaggtaaaacgtcatggacaaggattgtaacaaagatttgtccacatcccattcggacctcgctgtcacaccgttaatgattaccctcgatcgaatcgattcgacgaaggacaaaacatttgcgtatgtaagagctttcgcctggtatgttcgaatgtggttaagcactgtaaacacgaaatccttggacactaagacttccttgcgcgtcctcttactagtctccaaagaaatgtcgaataatggtacgatgaccatatccctcattttgggaaaccagtaattgactgatgatgaatcctcaaggaggattctctcgctgttgcacattgcaagagtctttttgtaatgccatgcgtcttccattgcagtataaaactgctcactatctacacttttatgggccacacctttgtacaaaagaaaagtatctattctagaaaacttacaaaaccaggtattaactctggtgactaaaaactccttcatgtaaacctctctattagaggccgggaagtaagttttgcacacatacttaagaatattagaataactatgacagtaattaagagtactctctgatgcaaaagaaaaggtcaacttgtctccatcgcgcgaaaaacacgcgttgatttcgtccaaattgacgcatgaatcttcaagaagcaggttctcggagaagtggaaagcggcatagcacgtatggacatttttcctcaagagtgccgccccgaactcatcggctggtatgtcatatatgctgtgtagcgcaatggcatacactctgcctgattgctgcatcggctgatgttcgcatgtctggaaagtattgtgacagacagcgtcttcaggaatttctgcgtatctgtcaaatgcttccttttggaagttggggactgttttcccccctctctctagcctagaaaggtatagttcaatactgtctttctggccttcgtgccgcatgatgtctcgaacgtccaggttgggcatgcagcagtgtacatatgctcgtcccttgaacagatgcgatgcaaaattcccgcctatgtcataagtcaatgatccgtagggaatttgcatcatcagatattccagttctaaagatcgcaatccacctgcaagcgaatgcacggcattttgcgtgttataaaatgtaatttggaattctggatacgcccgggtagcaataagcgtctgctcctcgcttattacttttgaaaagttcaccttgggcctgcggtcacgagcgttaaactcttcaaccgctgtgtcgtaaagacgacgctttgctagatcattgaccaaggagttgtttcctcggacagtgtccagcaaagctgatgtggtagctgtctgtgtgtatgccattgtaattgtaaatagtaattgtaatgttgtctgttgtttgttgttgttggtaattgttgtaaaaatac

SEQ ID NO: 32 - DNA, 25 bp, synthetic construct
  source 1..25: mol_type = other DNA
tgacaggatatattggcgggtaaac

SEQ ID NO: 33 - DNA, 25 bp, synthetic construct
  source 1..25: mol_type = other DNA
gtttacccgccaatatatcctgtca

SEQ ID NO: 34 - DNA, 21 bp, synthetic construct
  source 1..21: mol_type = other DNA
gatgattcggaggctactgtc

SEQ ID NO: 35 - DNA, 22 bp, synthetic construct
  source 1..22: mol_type = other DNA
cagaaggaccaggttgaagttg

SEQ ID NO: 36 - DNA, 22 bp, synthetic construct
  source 1..22: mol_type = other DNA
cctacctcaagttgcaggaccg

SEQ ID NO: 37 - DNA, 17 bp, synthetic construct
  source 1..17: mol_type = other DNA
tcgaggtcgacggtatc

(19) United States
(12) Patent Application Publication
(10) Pub. No.: US 2025/0304922 A1
(43) Pub. Date: Oct. 2, 2025

(54) METHODS AND COMPOSITIONS OF VIRUS-LIKE PARTICLES OF CYTOPLASMIC TYPE CITRUS LEPROSIS VIRUS FOR NANOTECHNOLOGY APPLICATIONS

(71) Applicant: The Regents of the University of California, Oakland, CA (US)

(72) Inventors: Nicole F. Steinmetz, San Diego, CA (US); Oscar A. Ortega-Rivera, Concord, CA (US)

(21) Appl. No.: 19/109,518

(22) PCT Filed: Sep. 6, 2023
(86) PCT No.: PCT/US2023/073593

§ 371 Date: Mar. 6, 2025

Related U.S. Application Data
(60) Provisional application No. 63/404,469, filed on Sep. 7, 2022.

(51) Int. Cl. C12N 7/00; C07K 16/08; C12N 15/113; C12N 15/82

(52) U.S. Cl. CPC C12N 7/00; C07K 16/08; C12N 15/113; C12N 15/8257; C12N 2310/11; C12N 2760/20022; C12N 2760/20023; C12N 2760/20051

(57) ABSTRACT

A nanoparticle comprising a cytoplasmic type citrus leprosis virus-like particle (CiLV-C) p29 protein coat and an optional agent encapsulated with the protein coat is provided herein. The nanoparticles are useful for the delivery of the agents to cells or tissues.

### CROSS-REFERENCE TO RELATED APPLICATIONS

This application claims priority under 35 U.S.C. § 119 (e) to U.S. Provisional Application No. 63/404,469, filed Sep. 7, 2022, the contents of which are incorporated herein by reference in its entirety.

### STATEMENT OF GOVERNMENT SUPPORT

This invention was made with government support under CA153915 and CA218292 awarded by the National Institutes of Health, and under DMR2011924 awarded by the National Science Foundation. The government has certain rights in the invention.

### SEQUENCE LISTING

The instant application contains a Sequence Listing which has been submitted electronically in XML format and is hereby incorporated by reference in its entirety. Said XML copy, created on Sep. 6, 2023, is names 114198-4560_SL.xml and is 76.5 kilobytes in size.

### BACKGROUND

Plant viruses are widely used as platform technologies and nanoparticles that can be repurposed and engineered for diverse applications, including the investigation of viral assembly mechanisms [1], the development of nanocontainers for catalysts or drugs [2], precision farming [3, 4] as well as veterinary [5] and human health [6]. They are particularly promising as vaccine and immunotherapy platforms, including a COVID-19 vaccine candidate based on tobacco mosaic virus (TMV) produced by Kentucky BioScience International, LLC [7]. Many applications have focused on TMV, which is a 300×18 nm hollow nanotube in its native state but can also form icosahedrons in certain environments. Many different icosahedral plant viruses have been studied and engineered, all of which form 30-nm particles with T=3 or pT=3 symmetry. Examples include cowpea mosaic virus (CPMV) [8], cowpea chlorotic mottle virus (CCMV) [9], cucumber mosaic virus (CuMV) [10], red clover necrotic mottle virus (RCNMV) [4], and hibiscus chlorotic ringspot virus (HCRSV) [11].

### SUMMARY OF THE DISCLOSURE

Cytoplasmic type citrus leprosis virus (CiLV-C) causes citrus leprosis, a viral disease of citrus crops that is prevalent in South and Central America [13]. The disease is transmitted when plants are infested with mites (Brevipalpus spp.) and is caused by at least three viruses (CiLV-C being one of them), which establish non-systemic infections characterized by chlorotic lesions with necrotic ringspots on leaves, and chlorotic lesions and/or browning of fruits [13]. The disease results in fruit loss, stem dieback and in severe infestations can even kill citrus trees. The combined cost of yield losses and chemical control measures for the prevention of mite infestations come to more than US$100 millions per year [14]. CiLV-C is the most widely distributed of the three viruses and is the type member of the genus Cilevirus, family Kitaviridae [15, 16]. Its bacilliform particles surround a positive-sense ssRNA genome in two segments, each featuring a 5′ cap and 3′ polyadenylate tail. The first segment (RNA1) contains two open reading frames (ORFs) encoding a multi-domain replication-associated protein and the capsid protein, p29 [13, 17, 18]. The second segment (RNA2) contains four ORFs encoding p15, which is required for the formation of vesicles in the ER [18], the p61 glycoprotein with roles in the remodeling of the ER and Golgi body [18, 19], the movement protein p32, and the integral membrane protein p24, which is also involved in viral replication and assembly in the ER and may function as a matrix protein [18].

Many plant virus-like particles (VLPs) utilized in nanotechnology are 30-nm icosahedrons. As described herein, Applicant produced VLPs of Cytoplasmic type citrus leprosis virus (CiLV-C) in Nicotiana benthamiana. CiLV-C that have a unique bacilliform shape (60-70 nm×110-120 nm). In one aspect, the CiLV-C capsid protein (p29) gene was transferred to the pTRBO expression vector (see FIG. 7) transiently expressed in leaves. Stable VLPs were formed, as confirmed by agarose gel electrophoresis, transmission electron microscopy and size exclusion chromatography. Interestingly, the morphology of the VLPs (15.8±1.3 nm icosahedral particles) differed from that of the native bacilliform particles indicating that the assembly of native virions is influenced by other viral proteins and/or the packaged viral genome. In addition to the disclosed therapeutic and agricultural applications, the smaller CiLV-C VLPs are also useful for structure-function studies to compare with the 30-nm icosahedrons of other VLPs.

Thus, this disclosure provides a nanoparticle comprising a cytoplasmic type citrus leprosis virus-like particle (CiLV-C) p29 protein coat or its equivalent and an optional agent, e.g. a therapeutic agent or an agricultural agent such as a pesticide, encapsulated with the protein coat. In one aspect, the CiLV-C virus like-particle of the nanoparticle lacks the p61 glycoprotein or its equivalent and the integral membrane protein p24 or its equivalent. In another aspect, the CiLV-C virus-like particle of the nanoparticle further comprises the CiLV-C movement protein p32 or its equivalent or a tobacco mosaic virus (TMV) movement protein or its equivalent.

The nanoparticles can further comprise a detectable label.

Also provided is a composition comprising the nanoparticle as described herein and a carrier, such as a pharmaceutically acceptable carrier.

Further provided is population of any of the nanoparticles as described therein, wherein the nanoparticles of the population or plurality can be the same or different from each other. The plurality of the nanoparticles in the population comprise VLP-p29 protein or its equivalent and optionally, an agent such as a therapeutic agent or an agricultural agent such as a pesticide encapsulated within the protein coat of the nanoparticle(s) of the plurality.

The nanoparticles of the population can be detectably labeled.

Also provided is a composition comprising the population of nanoparticles as described herein and a carrier, such as a pharmaceutically acceptable carrier.

The nanoparticles are useful to deliver an agent such as a therapeutic agent or an agricultural agent such as a pesticide to an agricultural product. In one aspect, a method comprises, or consists essentially of, or yet further consists of comprising contacting the cell or agricultural produce with one or more of the CiLV-C virus-like nanoparticle, the population, the plurality, or the composition as described herein. The contacting can be in vitro or in vivo. In one aspect, the cell is a plant cell, and the nanoparticle optionally contains or comprises an agricultural agent such as a pesticide. In another aspect the cell is an animal cell, e.g., a mammalian or human cell, and the nanoparticle comprises a therapeutic agent to treat or prevent a disease or condition.

Further provided is a method to deliver a therapeutic agent to a subject in need thereof, comprising, or consisting essentially thereof, or consisting of administering to the subject the CiLV-C virus-like particle, population or composition as described herein the nanoparticle(s) containing the therapeutic agent.

Also provided is a recombinant polynucleotide encoding the nanoparticle as described above, which in one aspect comprises, or consists essentially of, or yet further consists of: an expression vector; a TMV replicase; a polynucleotide encoding a CiLV-C movement protein p32 or its equivalent or a TMV movement protein or its equivalent. The polynucleotide can be RNA or DNA. Further provided is an organism or host cell comprising this recombinant polynucleotide. In one aspect, the organism comprises Brevipalpus spp. or Agrobacterium tumefaciens. In a further aspect, the host cell comprises N. benthamiana. The recombinant polynucleotide can further comprise a detectable label.

This disclosure also provides the CiLV-C virus particle produced by expressing the polynucleotide of this disclosure in an organism infected plant cell. In one aspect, the organism comprises A. tumefaciens and the plant cell comprises N. benthamiana.

A method to package a therapeutic agent or pesticide is provided, the method comprising, or consisting essentially of, or yet consisting of contacting the CiLV-C virus nanoparticle as described herein with the therapeutic agent or an agricultural agent such as a pesticide. In one aspect, the therapeutic agent comprises a polynucleotide, such as DNA or RNA. In a further aspect, the CiLV-C virus particle is isolated from the plant cell, a plant or a plant cell culture.

### BRIEF DESCRIPTION OF THE FIGURES

FIGS. 1A-1C: Cloning of the CiLV-C p29 gene in a plant expression vector. FIG. 1A: The p29 gene (an optimized p29 polynucleotide is provided in FIG. 6B, SEQ ID NO: 1) was transferred from its source vector pUC57-mini_P29_CiLVC to the pTRBO (tobacco mosaic virus RNA-based overexpression, see FIGS. 7A and 7B) vector using the restriction enzymes PacI and AvrII, yielding the final vector pTRBO_P29_CiLVC (see FIG. 7). FIG. 1B: Digestion of pUC57-mini_P29_CiLVC with PacI and AvrII. releasing the p29 gene as an 813-bp fragment. The same enzymes were used to linearize pTRBO to allow unidirectional ligation. FIG. 1C: Following the electroporation of A. tumefaciens strain GV3101 with pTRBO_P29_CiLVC. the presence of the insert was confirmed by colony PCR to yield a 307-bp product. and by a diagnostic restriction digest to release the entire 813-bp insert along with the >10-kb linearized pTRBO backbone.

FIGS. 2A-2E: Purification and analysis of CiLV-C VLPs. FIGS. 2A-2C: Schematic illustration of the sucrose cushion ultracentrifugation step. FIG. 2A: The N. benthamiana leaf extract was loaded onto the 30% sucrose cushion. FIG. 2B: After ultracentrifugation, the CiLV-C VLPs formed a band at the top of the sucrose cushion and most plant proteins either remained in the aqueous zone or formed a pellet. FIG. 2C: Image of a tube after ultracentrifugation showing the CiLV-C VLP band. FIG. 2D: Analysis of 20 μg of total protein from the crude extract (left). the VLP band (middle) and the pellet (right) by 1.2% agarose gel electrophoresis. The top image shows nucleic acid staining (GelRed) and the bottom image shown protein staining (Coomassie Brilliant Blue) confirming the comigration of protein and nucleic acid in intact VLPs. FIG. 2E: Denaturing SDS-PAGE in 4-12% polyacrylamide gels of 10 and 20 μg of total protein from the crude extract (left). the VLP band (middle) and the pellet (right), indicating the presence of p29 in the crude extract and VLPs but not the pellet.

FIGS. 3A-3B: Characterization of CiLV-C VLP size and morphology by transmission electron microscopy (TEM). FIG. 3A: TEM images of negatively-stained CiLV-C VLPs (scale bar=100 nm). FIG. 3B: Particle size and distribution determined by analyzing TEM images using ImageJ software. PDI=polydispersity index.

FIGS. 4A-4D: Purification of CiLV-C VLPs by size exclusion chromatography (SEC) and characterization of the fractions. FIG. 4A: SEC revealed one major peak and two minor peaks among five fractions. FIG. 4B: SDS-PAGE analysis in 4-12% polyacrylamide gels under denaturing conditions reveals the presence of the p29 protein in the fractions representing all three peaks. with the most abundant protein in fraction #4, representing the major peak. FIG. 4C: TEM images from fraction #4 showing pure CiLV-C VLPs (49.000×. scale bar=100 nm). FIG. 4D: TEM images from fraction #4 showing pure CiLV-C VLPs (128.000×, scale bar=50 nm).

FIG. 5: The production and testing of cytoplasmic type citrus leprosis virus-like particles (CiLV-C VLPs) began with the introduction of the p29 gene (shown in FIG. 6B, SEQ ID NO: 1) encoding the CiLV-C capsid protein into the pTRBO vector to form pTRBO_P29_CiL VC, which was introduced into Agrobacterium tumefaciens strain GV3101 (left panel). Transient expression in Nicotiana benthamiana leaves was then facilitated by vacuum-assisted agroinfiltration (middle panel). After 7 days, the VLPs were extracted from detached leaves for testing. Abbreviations: LB=left border; RB=right border; replicase/MP=the RNA-dependent RNA polymerase and movement protein from tobacco mosaic virus; P29=p29 gene encoding the 29-kDa CiLV-C capsid protein.

FIGS. 6A-6D: Maps and sequence of the 823 base pair CiLV (identified as opP29_CILVC) construct containing the p29 gene encoding the capsid protein (amino acids 1-268, SEQ ID NO: 1). FIG. 6A is a map of the construct showing termini of the location of the elements of the construct. FIG. 6B is the polynucleotide (SEQ ID NO: 3) and amino acid sequence (SEQ ID NO: 2) of the construct, including the sequences of the p29 gene and polypeptide. FIGS. 6C and 6D show restriction enzyme locations along the construct.

FIGS. 7A-7D: Map and sequence of the pTRBO_29_CiL VC expression plasmid. FIG. 7A is the restriction enzyme map of the pTRBO_29_CiLVC plasmid (11,414 base pairs). FIG. 7B shows the polynucleotide sequence encoding at least the following identified regions of the pTROBO_29 plasmid, including TMVQ, (SEQ ID NO: 3) Replicase (SEQ ID NO: 4), TMV movement protein (MP, polynucleotide (SEQ ID NO: 5 and polypeptide (SEQ ID NO: 6)), p29 gene (SEQ ID NO: 1) and p29 polypeptide (SEQ ID NO: 2). As shown in the figure, the expression plasmid comprises the polynucleotide (SEQ ID NO: 8) and encoded polypeptide (SEQ ID NO: 8)). FIG. 7C shows the restriction enzyme locations along the contruct. FIG. 7D is the annotation of the plasmid of FIG. 7B.

### DETAILED DESCRIPTION

### Definitions

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the disclosure to those skilled in the art. The terminology used in the description herein is for the purpose of describing particular embodiments only and is not intended to be limiting.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. While not explicitly defined below, such terms should be interpreted according to their common meaning.

The terminology used in the description herein is for the purpose of describing particular embodiments only and is not intended to be limiting of the disclosure. All publications, patent applications, patents and other references mentioned herein are incorporated by reference in their entirety.

Unless the context indicates otherwise, it is specifically intended that the various features of the disclosure described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless explicitly indicated otherwise, all specified embodiments, features, and terms intend to include both the recited embodiment, feature, or term and biological equivalents thereof.

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

Throughout this disclosure, various publications, patents and published patent specifications are referenced by an identifying citation or by an Arabic numeral. The full citation for the publications identified by an Arabic numeral are in the attached Appendix. The disclosures of these publications, patents and published patent specifications are hereby incorporated by reference into the present disclosure in their entirety to more fully describe the state of the art to which this disclosure pertains.

The practice of the present technology will employ, unless otherwise indicated, conventional techniques of organic chemistry, pharmacology, immunology, molecular biology, microbiology, cell biology and recombinant DNA, which are within the skill of the art. See, e.g., Sambrook, Fritsch and Maniatis, Molecular Cloning: A Laboratory Manual, 2nd edition (1989); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, a Laboratory Manual, and Animal Cell Culture (R. I. Freshney, ed. (1987)).

As used in the description of the disclosure and the appended claims, the singular forms “a,” “an” and “the” are intended to include the plural forms as well, unless the context clearly indicates otherwise.

The term “about,” as used herein when referring to a measurable value such as an amount or concentration and the like, is meant to encompass variations of 20%, 10%, 5%, 1%, 0.5%, or even 0.1% of the specified amount.

As used herein, the term “comprising” is intended to mean that the compositions or methods include the recited steps or elements, but do not exclude others. “Consisting essentially of” shall mean rendering the claims open only for the inclusion of steps or elements, which do not materially affect the basic and novel characteristics of the claimed compositions and methods. “Consisting of” shall mean excluding any element or step not specified in the claim. Embodiments defined by each of these transition terms are within the scope of this disclosure.

The terms or “acceptable,” “effective,” or “sufficient” when used to describe the selection of any components, ranges, dose forms, etc. disclosed herein intend that said component, range, dose form, etc. is suitable for the disclosed purpose.

Also as used herein, “and/or” refers to and encompasses any and all possible combinations of one or more of the associated listed items, as well as the lack of combinations when interpreted in the alternative (“or”).

As used herein, the term “isolated cell” generally refers to a cell that is substantially separated from other cells of a tissue. The term includes prokaryotic and eukaryotic cells.

“Eukaryotic cells” comprise, or alternatively consist essentially of, or yet further consist of all of the life kingdoms except monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. Unless specifically recited, the term “host” includes a eukaryotic host, including, for example, yeast, higher plant, insect and mammalian cells. Non-limiting examples of eukaryotic cells or hosts include simian, bovine, porcine, murine, rat, avian, reptilian and human,

“Prokaryotic cells” that usually lack a nucleus or any other membrane-bound organelles and are divided into two domains, bacteria and archaea. In addition to chromosomal DNA, these cells can also contain genetic information in a circular loop called on episome. Bacterial cells are very small, roughly the size of an animal mitochondrion (about 1-2 μm in diameter and 10 μm long). Prokaryotic cells feature three major shapes: rod shaped, spherical, and spiral. Instead of going through elaborate replication processes like eukaryotes, bacterial cells divide by binary fission. Examples include but are not limited to Bacillus bacteria, E. coli bacterium, and Salmonella bacterium.

A “composition” typically intends a combination of the active agent, e.g., the nanoparticle of this disclosure and a naturally occurring or non-naturally occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

The compositions used in accordance with the disclosure, including cells, treatments, therapies, agents, drugs, agricultural agents, pesticides, and pharmaceutical or agricultural compositions can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The compositions are easily administered in a variety of dosage forms, such as the type of injectable solutions described herein.

As used herein, the terms “nucleic acid sequence” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

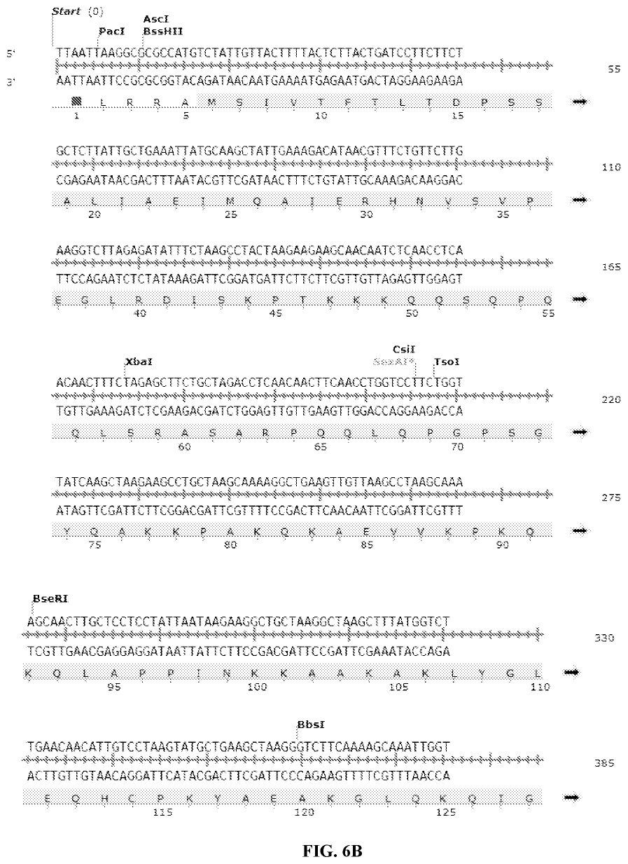

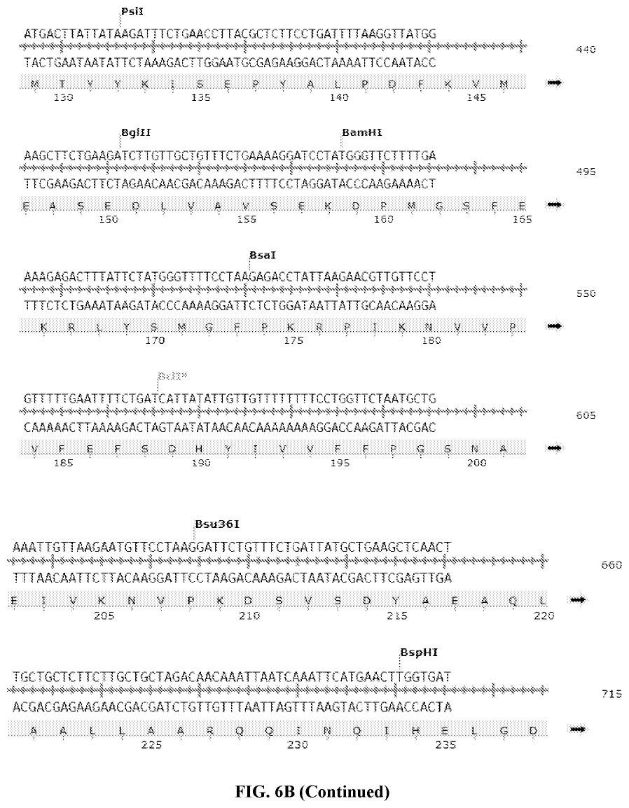

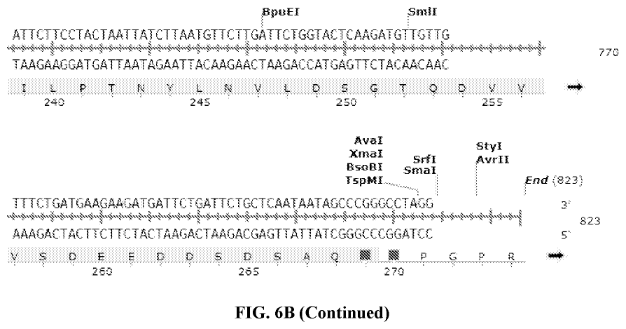

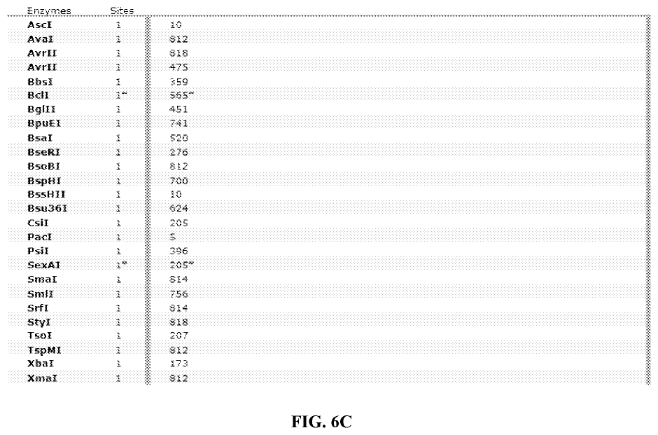

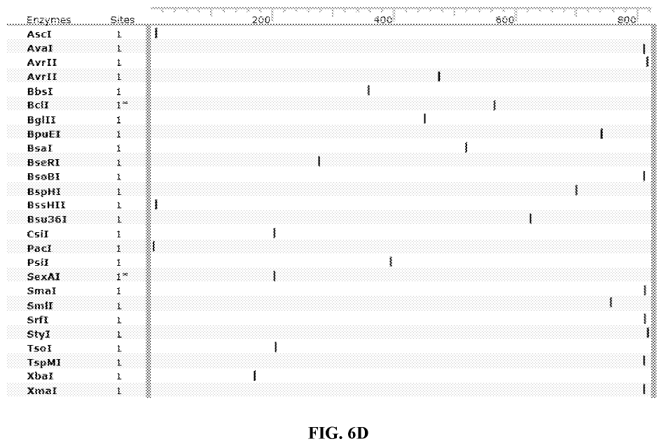

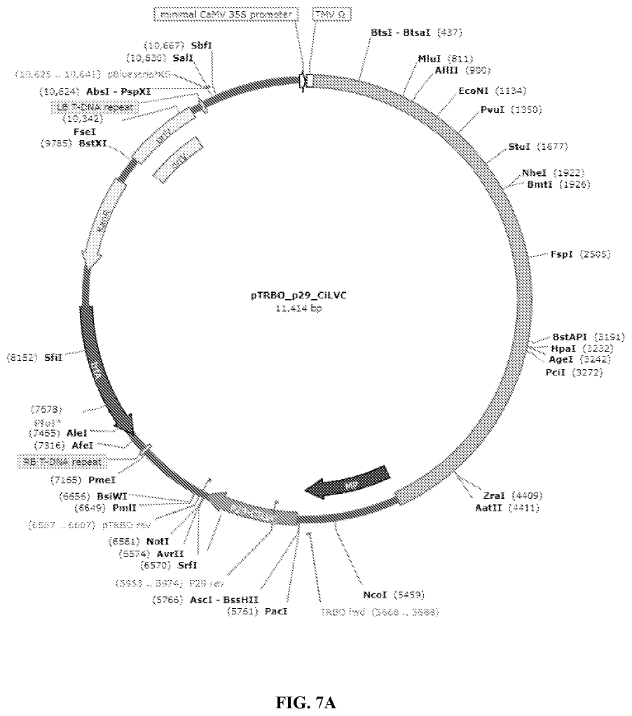

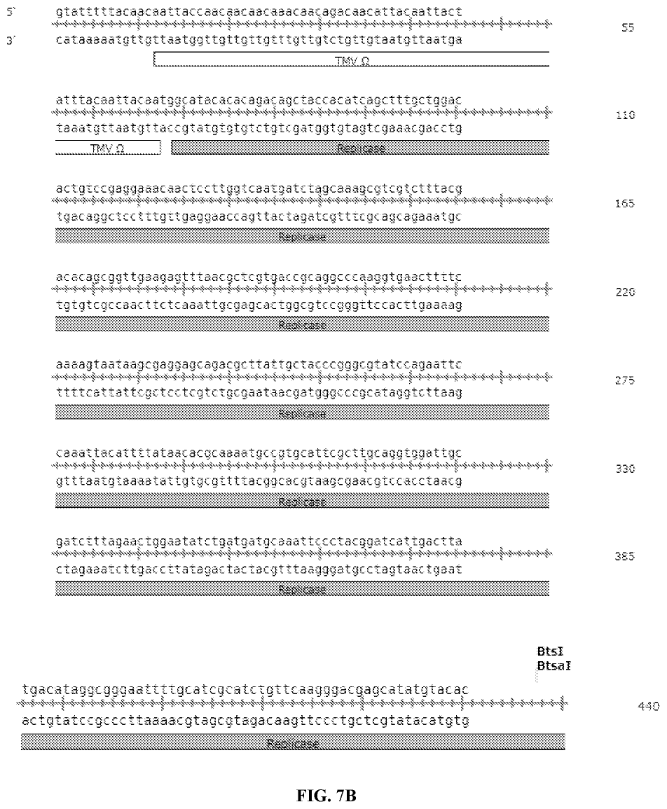

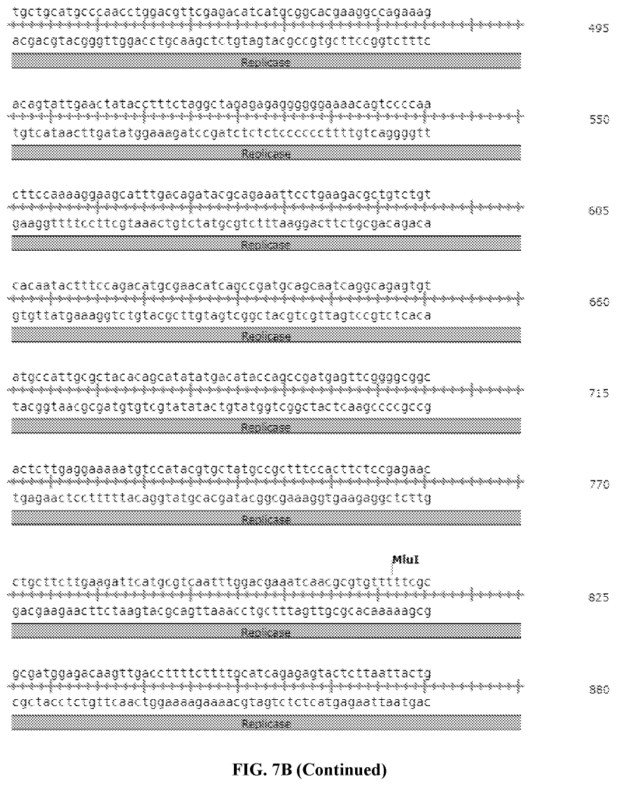

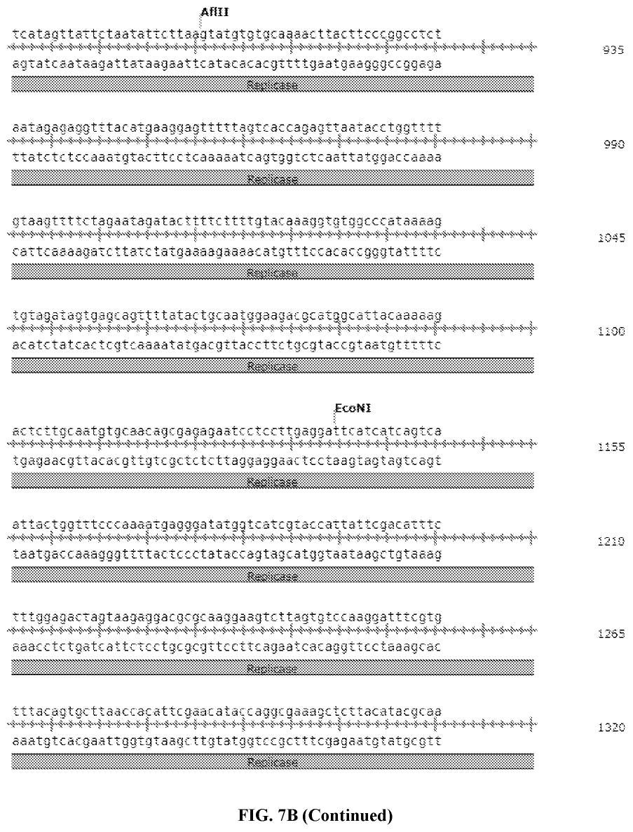

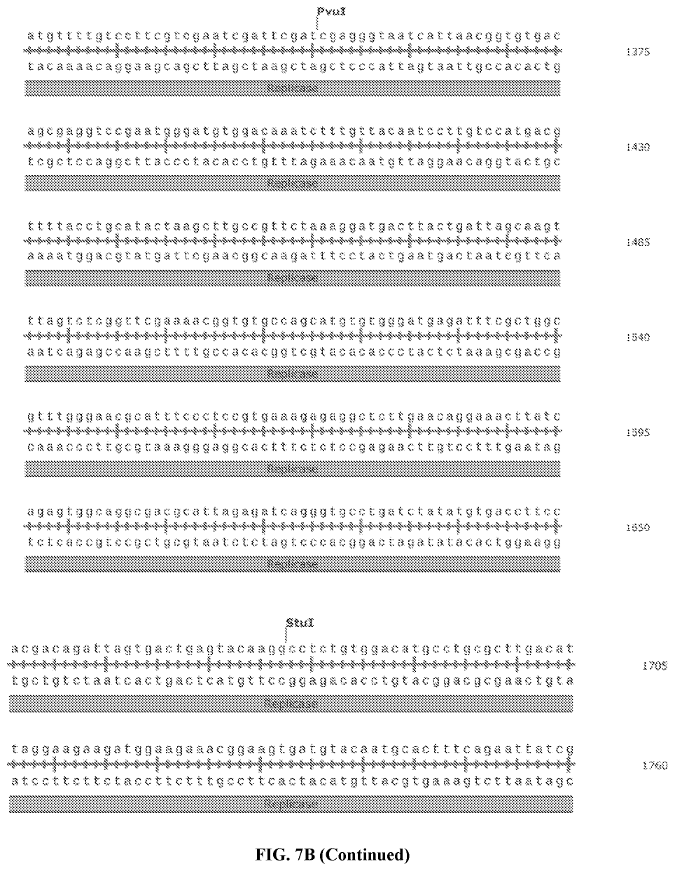

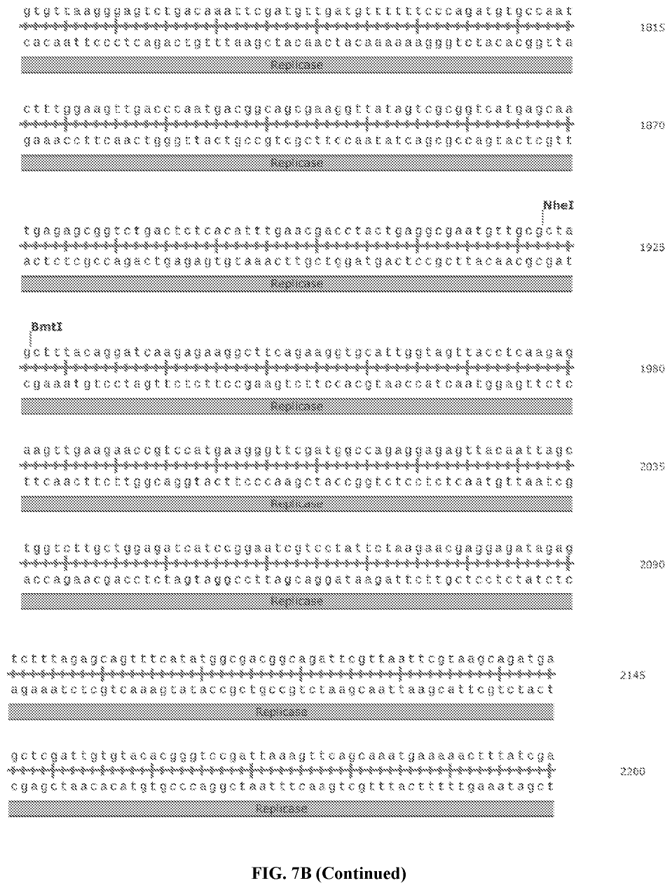

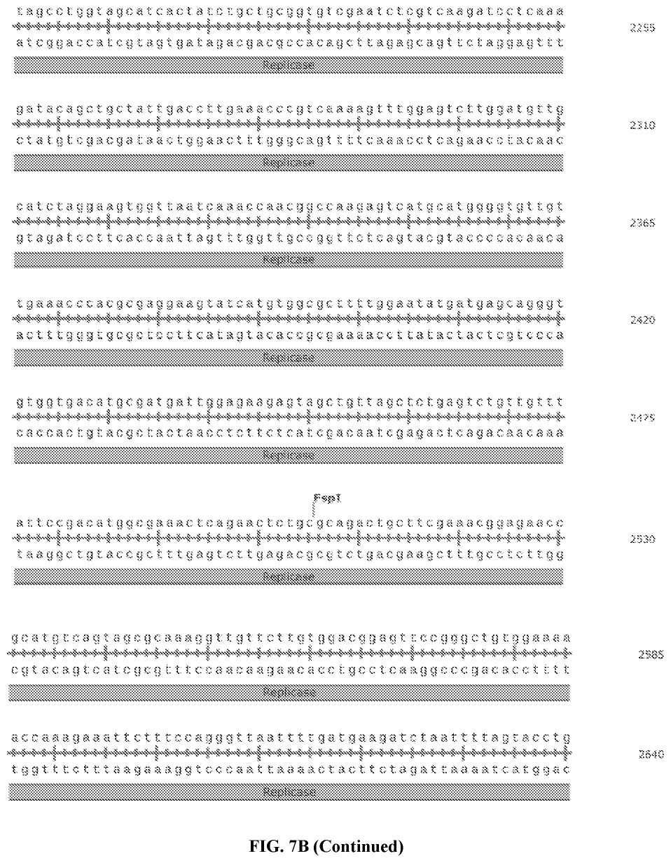

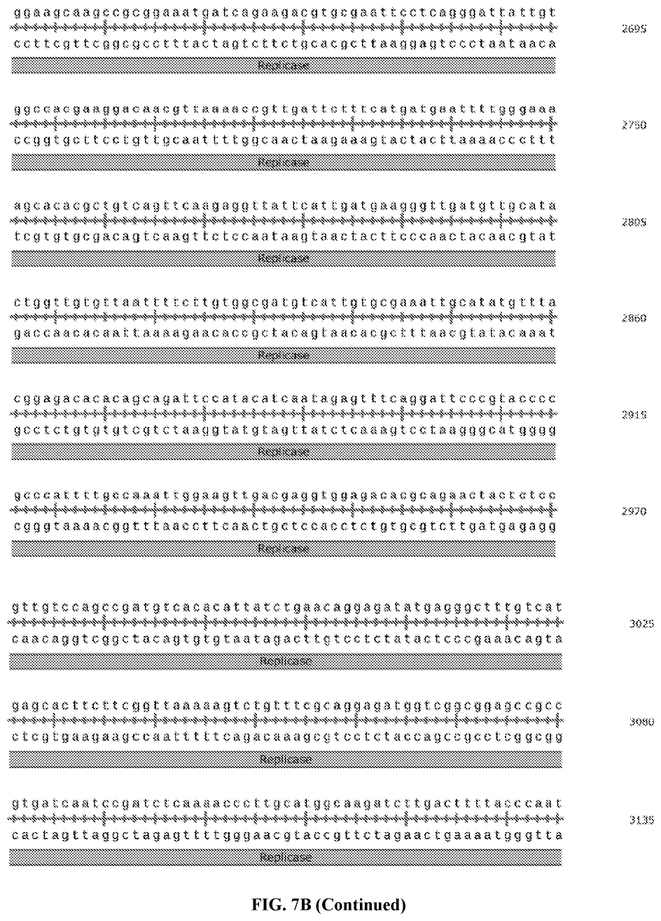

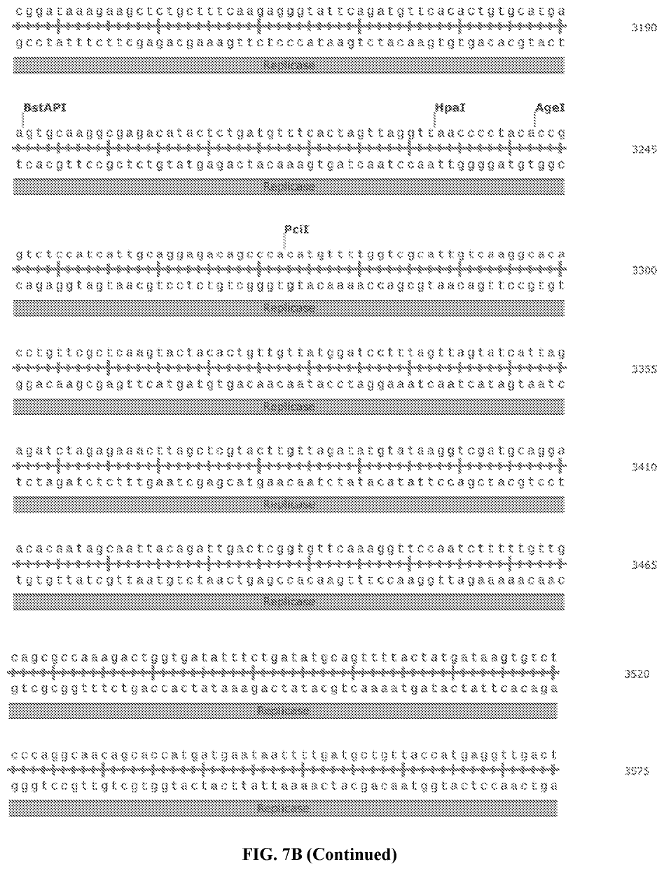

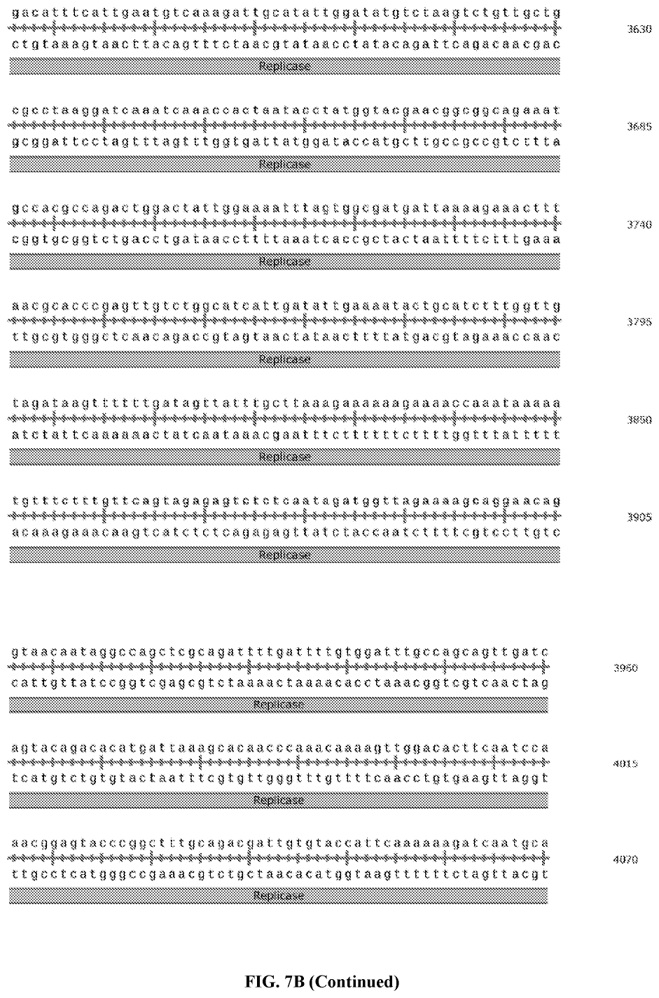

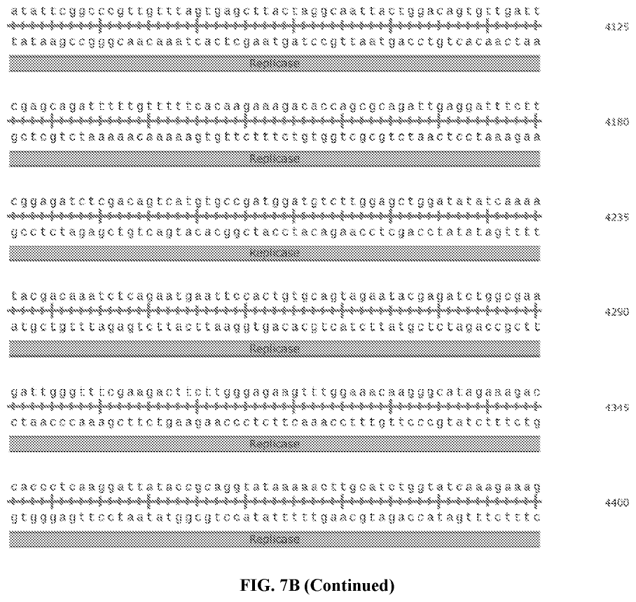

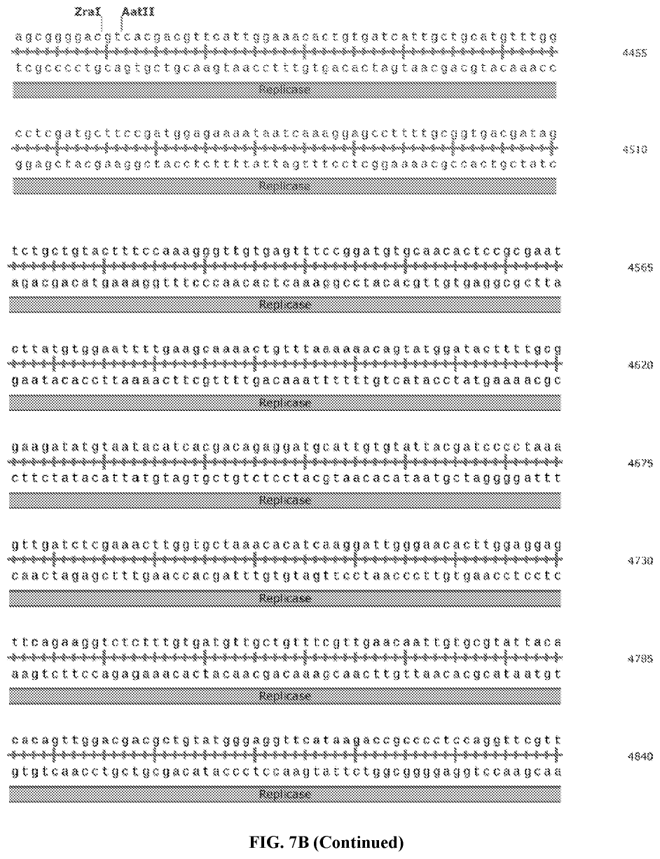

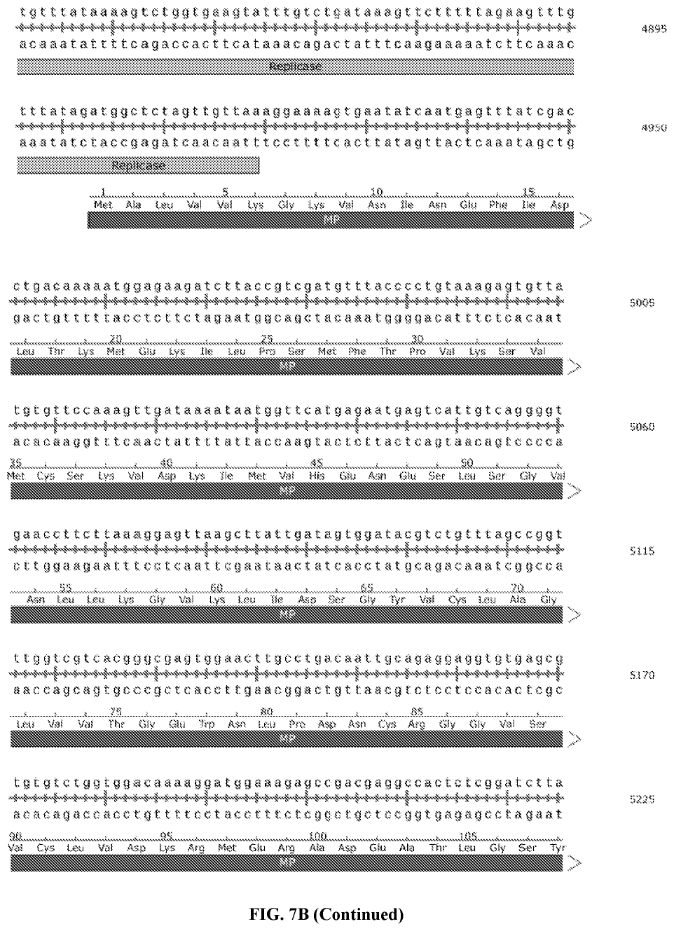

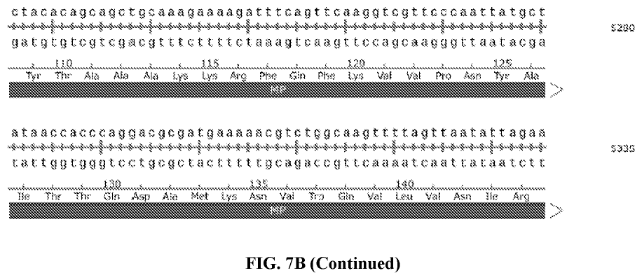

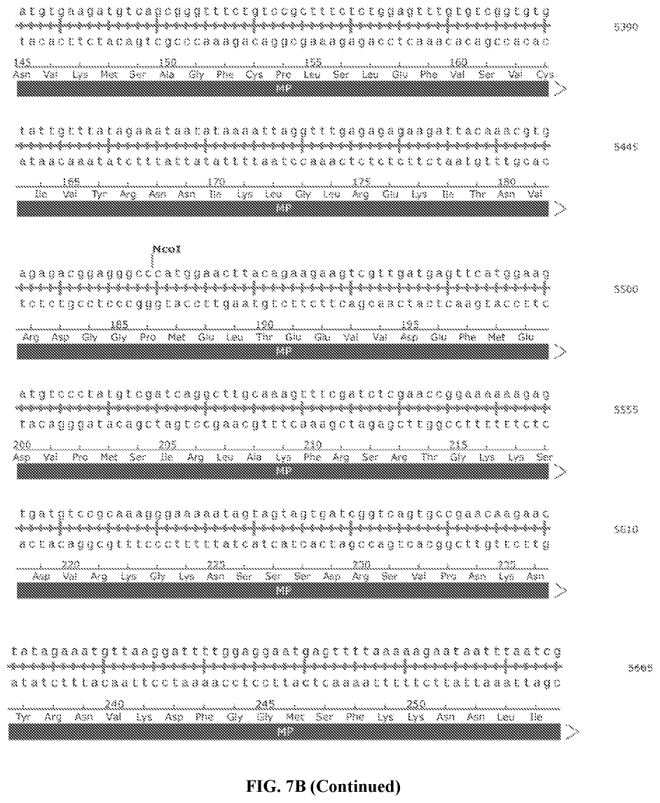

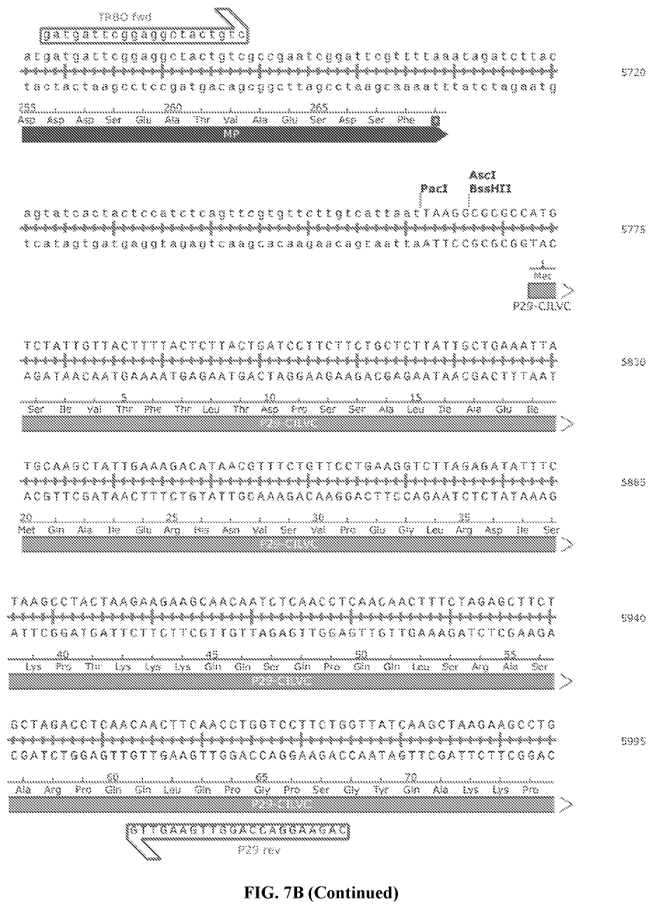

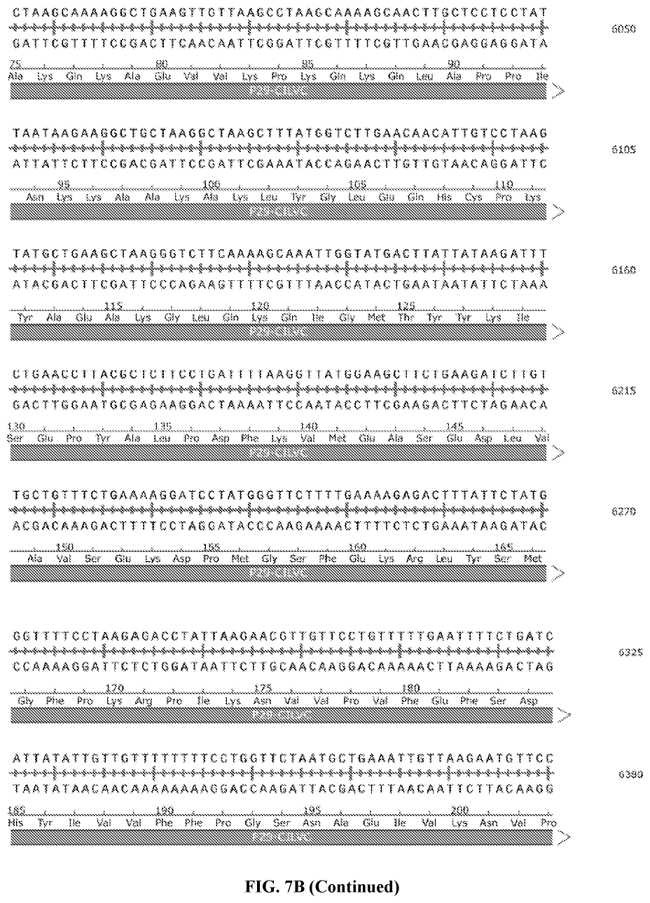

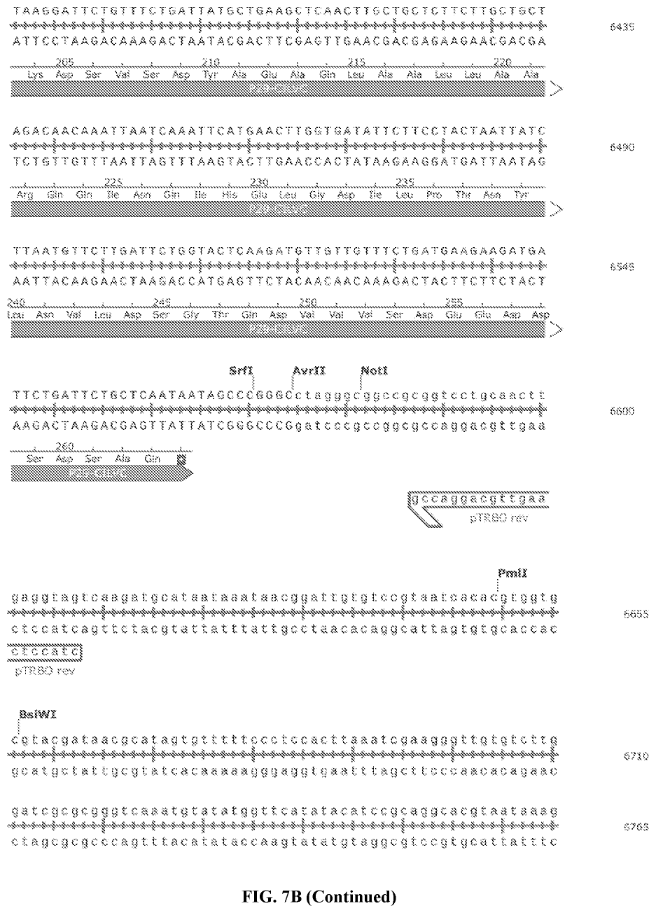

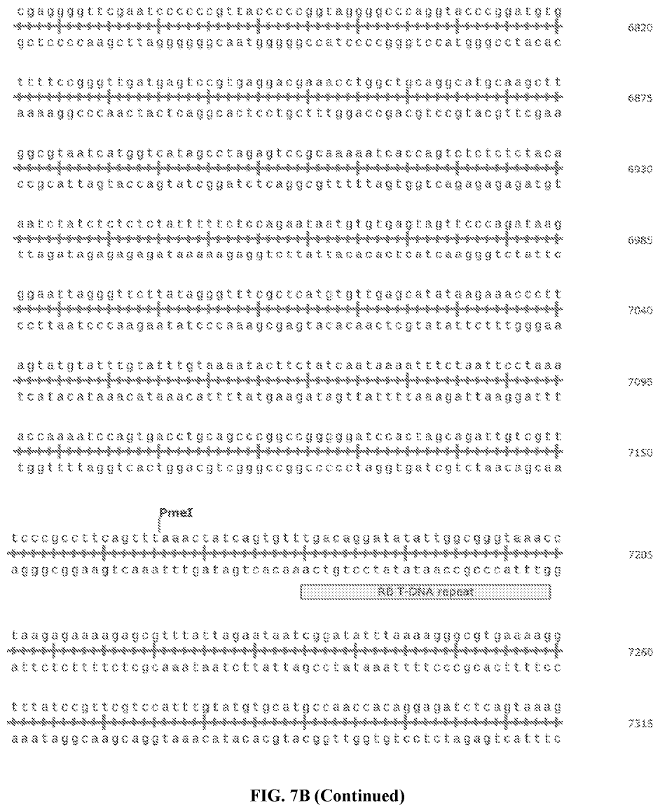

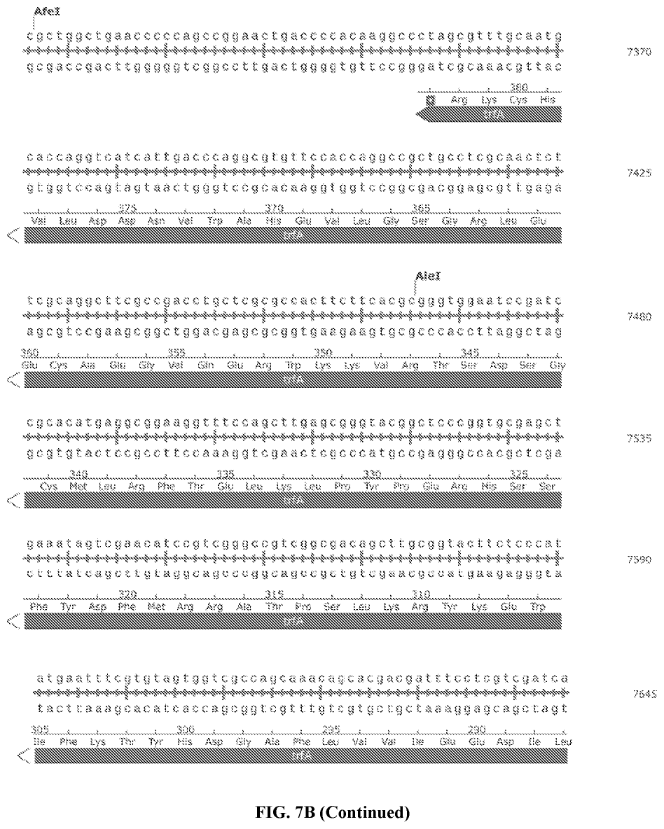

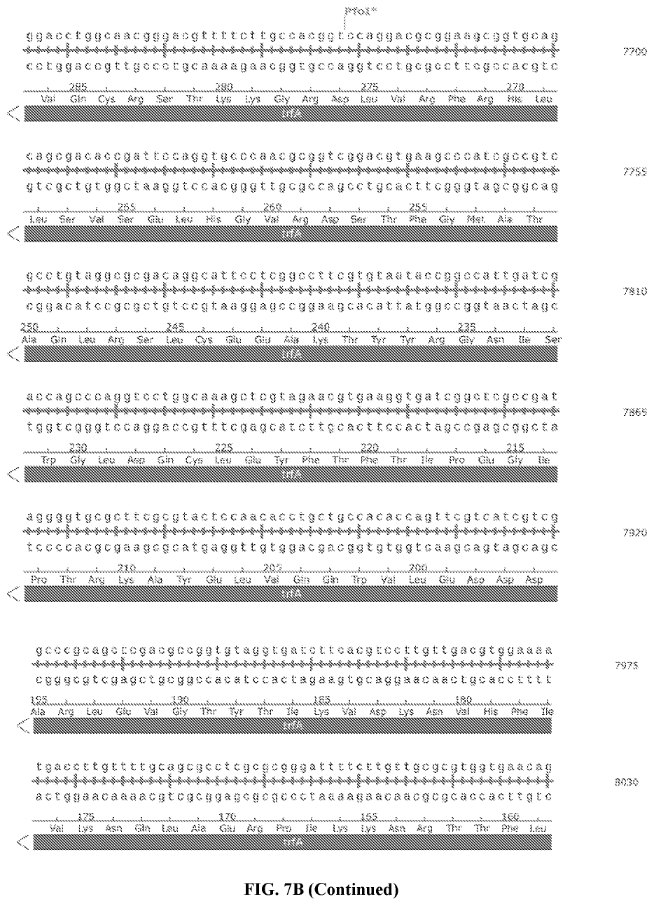

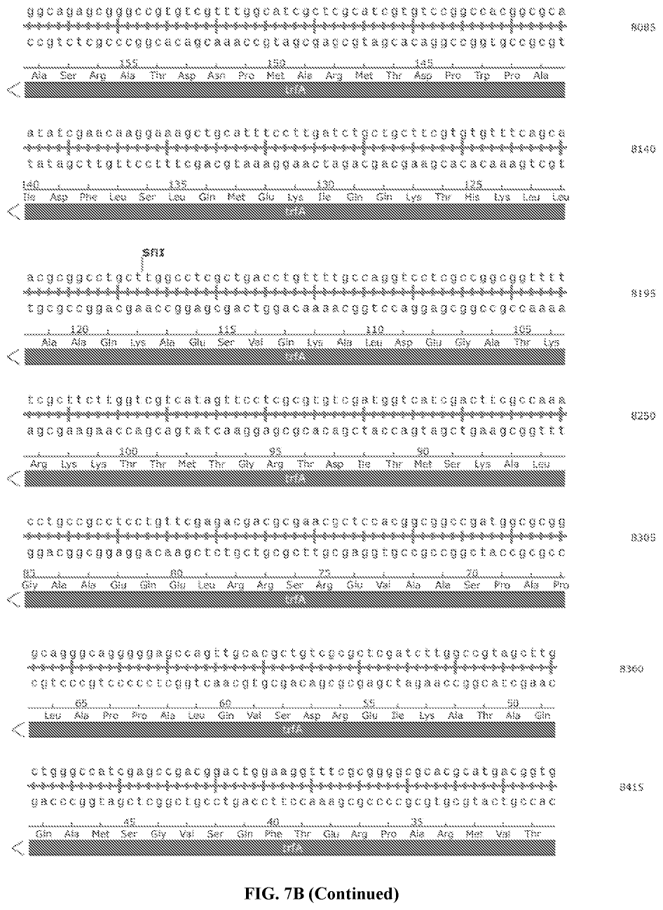

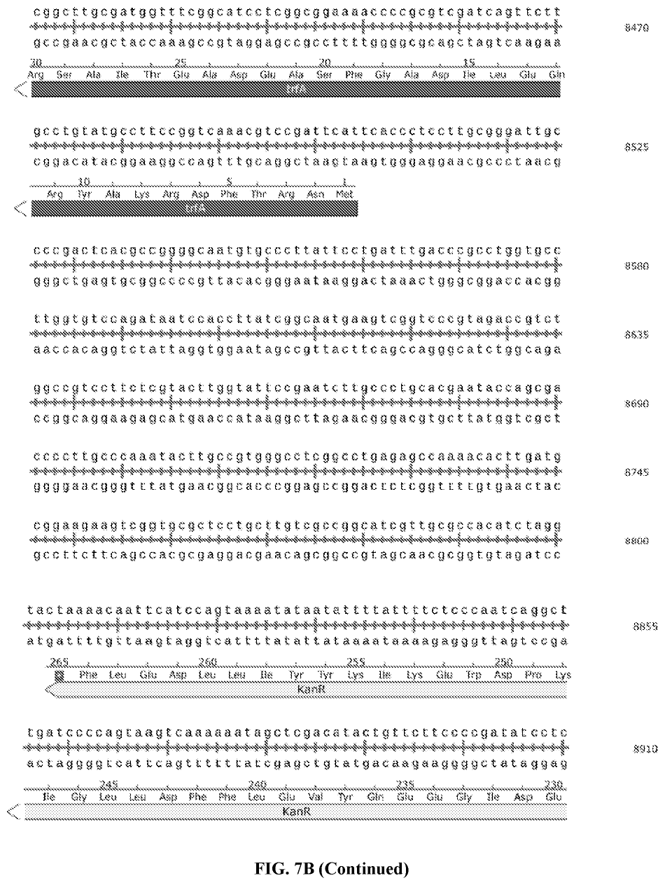

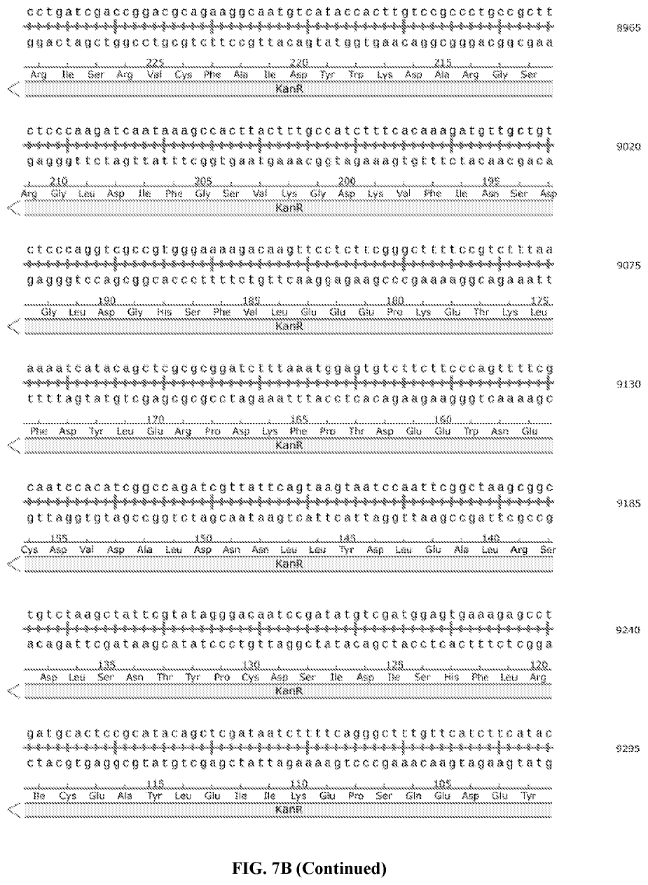

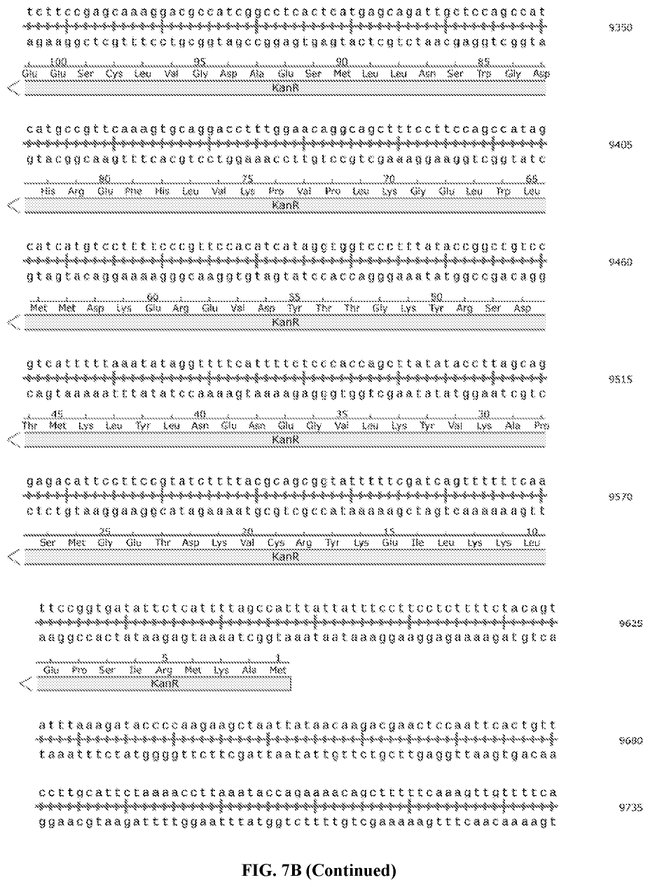

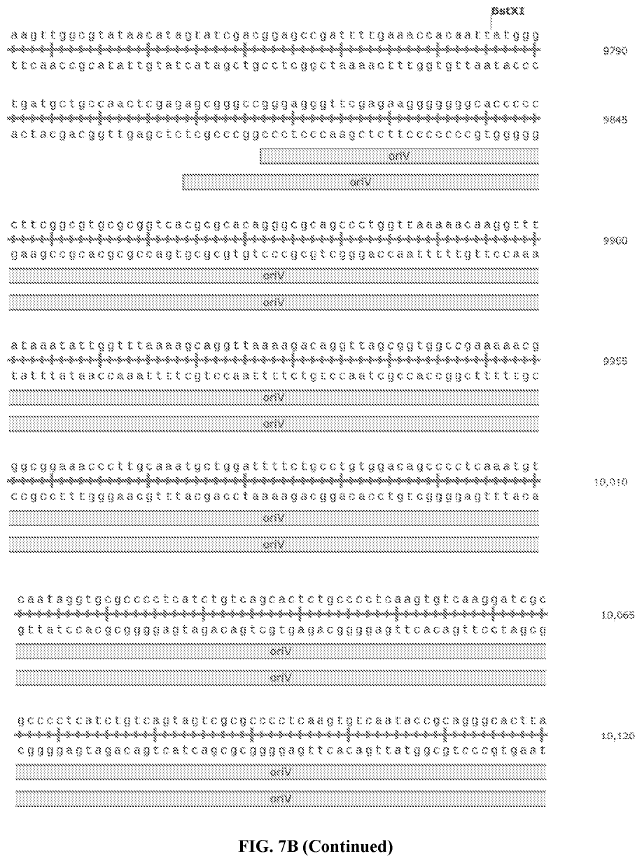

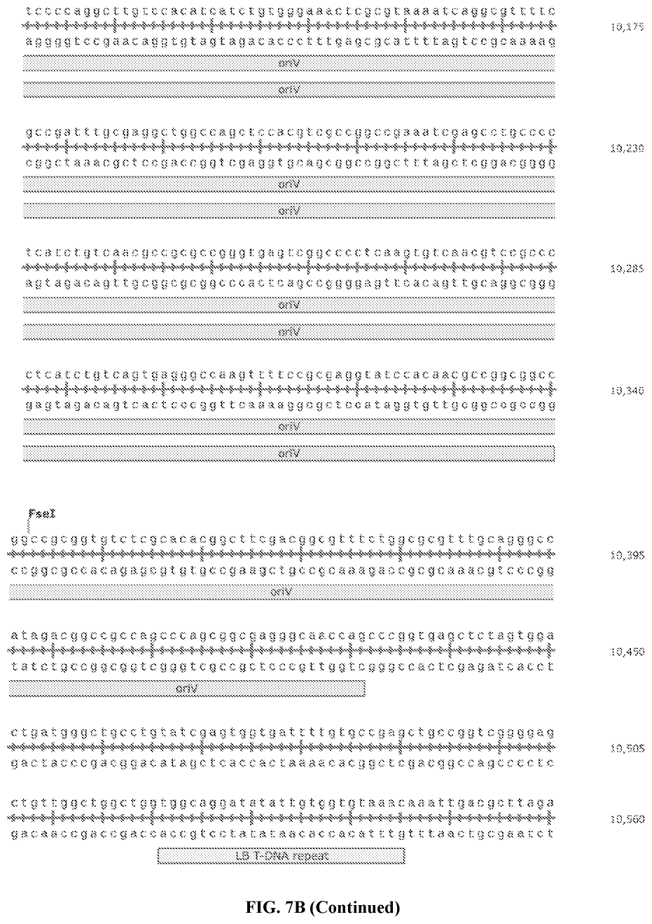

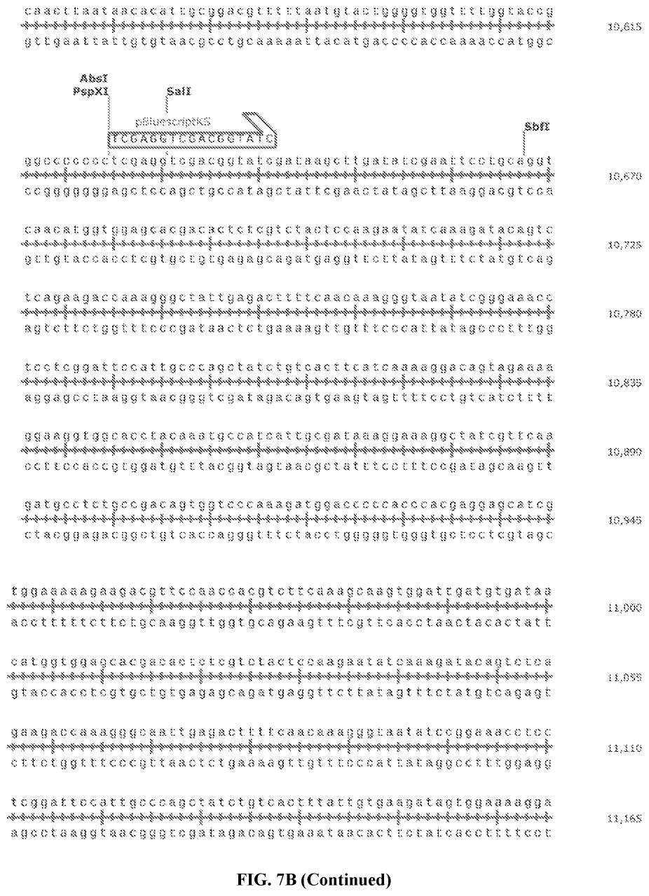

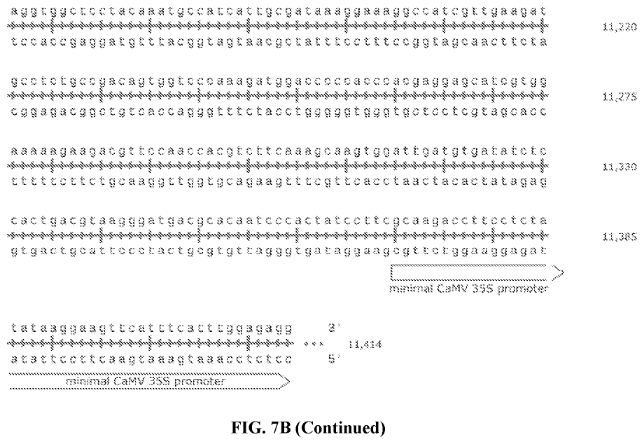

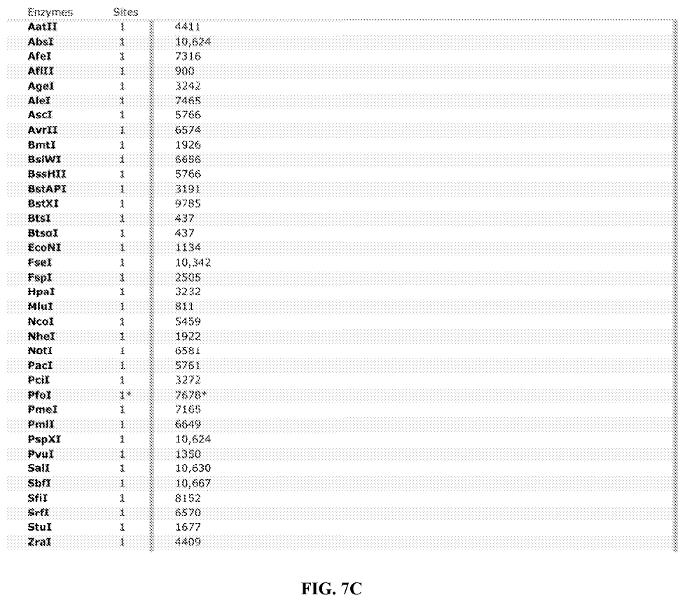

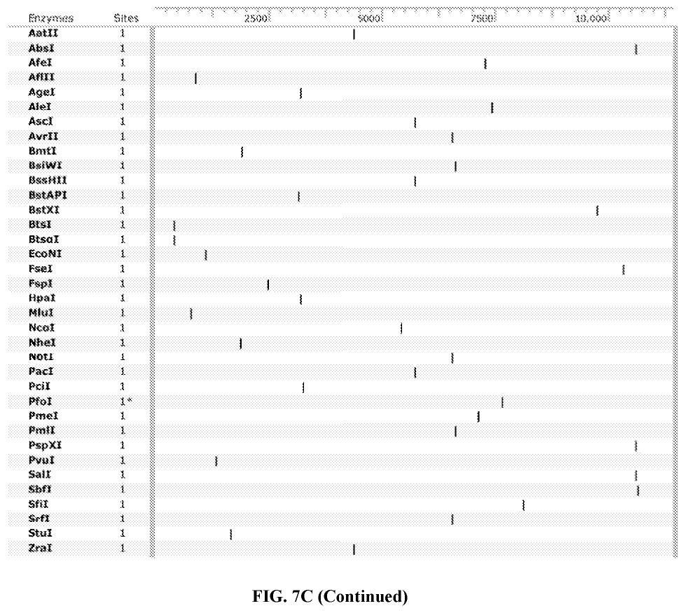

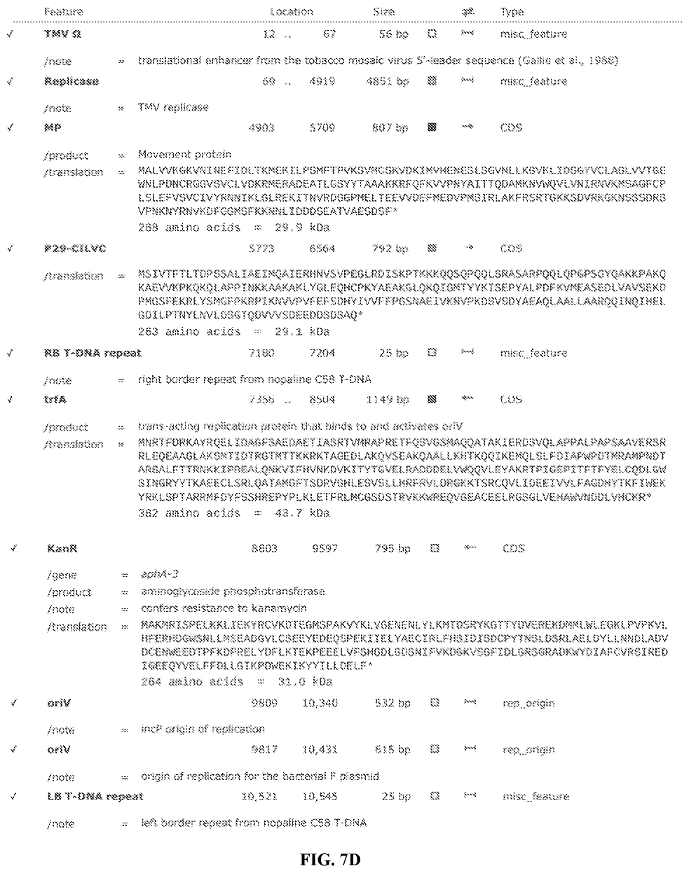

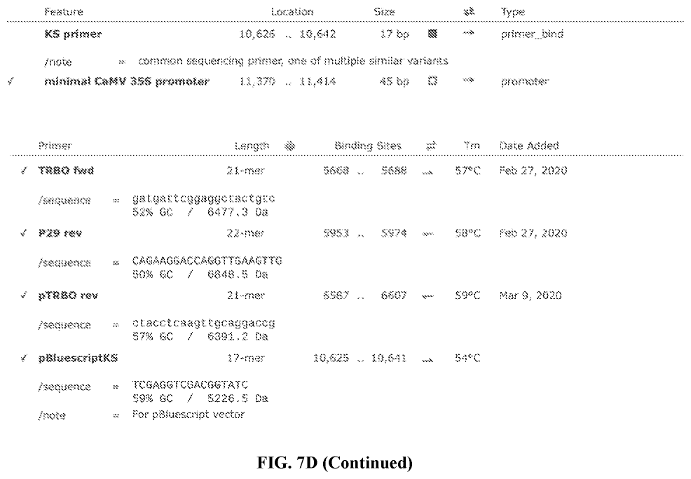

As used herein, the term “vector” refers to a nucleic acid construct deigned for transfer between different hosts, including but not limited to a plasmid, a virus, a cosmid, a phage, a BAC, a YAC, etc. In one aspect, the vector is a plant expression vector such as pUC57, a sequence of which is available at and commercially available from Genscript (www.genscript.com/vector/SD1176-pUC57_plasmid_DNA.html, last accessed on Sep. 2, 2023) or Thermofisher (see www.thermofisher.com/order/catalog/product/SD0171, last accessed on Sep. 2, 2023) or pTRBO (tobacco mosaic virus RNA-based overexpression, described for example in Lindbo, JA (2007) Plant Physiol. December; 145 (4): 1232-1340, and commercially available from Addgene, www.addgene.org/80083/, last accessed on Sep. 2, 2023). Additional examples of such vectors are provided in FIGS. 6 and 7.

A “viral vector” is defined as a recombinantly produced virus or viral particle that comprises a polynucleotide to be delivered into a host cell, either in vivo, ex vivo or in vitro. In some embodiments, plasmid vectors may be prepared from commercially available vectors. In other embodiments, viral vectors may be produced from baculoviruses, retroviruses, adenoviruses, AAVs, etc. according to techniques known in the art. In one embodiment, the viral vector is a lentiviral vector. Examples of viral vectors include retroviral vectors, adenovirus vectors, adeno-associated virus vectors, alphavirus vectors and the like. Further details as to modern methods of vectors for use in gene transfer may be found in, for example, Kotterman et al. (2015) Viral Vectors for Gene Therapy: Translational and Clinical Outlook Annual Review of Biomedical Engineering 17. Vectors that contain both a promoter and a cloning site into which a polynucleotide can be operatively linked are well known in the art. Such vectors are capable of transcribing RNA in vitro or in vivo and are commercially available from sources such as Agilent Technologies (Santa Clara, Calif.) and Promega Biotech (Madison, Wis.).

As used herein, the term “detectable label” intends a directly or indirectly detectable compound or composition that is conjugated directly or indirectly to the composition to be detected, e.g., polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g. radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to radioisotopes, fluorochromes, chemiluminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component.

Examples of luminescent labels that produce signals include, but are not limited to bioluminescence and chemiluminescence. Detectable luminescence response generally comprises a change in, or an occurrence of, a luminescence signal. Suitable methods and luminophores for luminescently labeling assay components are known in the art and described for example in Haugland, Richard P. (1996) Handbook of Fluorescent Probes and Research Chemicals (6th ed.). Examples of luminescent probes include, but are not limited to, aequorin and luciferases.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6^{th} ed.).

In another aspect, the fluorescent label is functionalized to facilitate covalent attachment to a cellular component present in or on the surface of the cell or tissue such as a cell surface marker. Suitable functional groups, including, but not are limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

Attachment of the fluorescent label may be either directly to the cellular component or compound or alternatively, can by via a linker. Suitable binding pairs for use in indirectly linking the fluorescent label to the intermediate include, but are not limited to, antigens/antibodies, e.g., rhodamine/anti-rhodamine, biotin/avidin and biotin/strepavidin.

An “effective amount” or “efficacious amount” refers to the amount of an agent or combined amounts of two or more agents, that, when administered for the treatment of a mammal or other subject, is sufficient to provide such treatment for the disease or purpose of the method. The “effective amount” will vary depending on the agent(s), the purpose of the method, the disease and its severity and the age, weight, etc., of the subject to be treated.

In some embodiments the effective amount will depend on the size and nature of the application in question. It will also depend on the nature and sensitivity of the target subject or plant and the methods in use. The skilled artisan will be able to determine the effective amount based on these and other considerations. The effective amount may comprise, or alternatively consist essentially of, or yet further consist of one or more administrations of a composition depending on the embodiment.

As used herein, the term “administer” or “administration” or “administering” intends to mean delivery of a substance to a subject such as an animal or human. Administration can be applied in one dose, continuously or intermittently throughout the course of treatment. Methods of determining the most effective means and dosage of administration are known to those of skill in the art and will vary with the composition used for method or therapy, the purpose of the method or therapy, as well as the age, health or gender of the subject being treated, plant or plant disease being treated. Single or multiple administrations can be carried out with the dose level and pattern being selected by the treating physician or in the case of pets and animals, treating veterinarian. Suitable dosage compositions and methods of administering the agents are known in the art. Route of administration can also be determined and method of determining the most effective route of administration are known to those of skill in the art and will vary with the composition used for treatment, the purpose of the treatment, the health condition or disease stage of the subject being treated and the target cell or tissue. Non-limiting examples of route of administration include intravenous, intra-arterial, intramuscular, intracardiac, intrathecal, subventricular, epidural, intracerebral, intracerebroventricular, sub-retinal, intravitreal, intraarticular, intraocular, intraperitoneal, intrauterine, intradermal, subcutaneous, transdermal, transmuccosal, and inhalation.

An “agricultural product” intends vegetation in whole or in part and includes plants, trees, roots, flowers, limbs, shoots, stems, leaves or any other part thereof.

As used herein, the terms “treating,” “treatment” and the like when used in a method for treating agricultural products mean obtaining a desired agricultural effect such as an amelioration. The effect may be prophylactic in terms of completely or partially preventing an infection of a plant, vegetation, or other agricultural product by a pest or insect or the deleterious result of such infection. In one aspect, the term “treatment” excludes prophylaxis.

The term “ameliorate” when treating an agricultural product means a detectable improvement in an agricultural product or vegetation, such as a plant, tree, flower, crop, root, stem, or leaf. A detectable improvement includes a subjective or objective decrease, reduction, inhibition, suppression, limit or control in the occurrence, frequency, severity, progression, or duration of an infection or presence of a pest or microorganism.

As used herein, the term “agricultural agent” intends an active agent that treats or ameliorate the presence of or infection of an agricultural product, e.g., a pesticide such as an insecticide. In one aspect, the agricultural agent is a nematicide selected from the group of Ivermectin, fluazaindolizine, fluensulfone, or fluopyram. In another aspect, the agricultural agent is an RNAi inducer such as an RNAi molecule such as small interfering RNAs (siRNA) that target nematodes, viruses, insects and the like.

Exemplary common pesticides for use in the nanoparticles of this disclosure are described in Table 1, below:

TABLE 1
Pesticide Insecticide Types and Their Modes of Action

| Pesticide Type | Mode of Action |
|---|---|
| Organochlorine | Most act on neurons by causing a sodium/potassium imbalance preventing |
| | normal transmission of nerve impulses. Some act on the GABA (γ- |
| | aminobutyric acid) receptor preventing chloride ions from entering the neurons |
| | causing a hyperexcitable state characterized by tremors and convulsions. |
| | Usually broad-spectrum insecticides that have been taken out of use. |
| Organophosphate | Cause acetylcholinesterase (AChE) inhibition and accumulation of |
| | acetylcholine at neuromuscular junctions causing rapid twitching of voluntary |
| | muscles and eventually paralysis. A broad-range insecticide, generally the most |
| | toxic of all pesticides to vertebrates. |
| Organosulfur | Exhibit ovicidal activity (i.e., they kill the egg stage). Used only against mites |
| | with very low toxicity to other organisms. |
| Carbamates | Cause acetylcholinesterase (AChE) inhibition leading to central nervous system |
| | effects (i.e., rapid twitching of voluntary muscles and eventually paralysis). Has |
| | very broad spectrum toxicity and is highly toxic to fish. |
| Formamidines | Inhibit the enzyme monoamine oxidase that degrades neurotransmitters causing |
| | an accumulation of these compounds; affected insects become quiescent and |
| | die. Used in the control of OP and carbamate-resistant pests. |
| Dinitrophenols | Act by uncoupling or inhibiting oxidative phosphorylation preventing the |
| | formation of adenosine triphosphate (ATP). All types have been withdrawn |
| | from use. |
| Organotins | Inhibit phosphorylation at the site of dinitrophenol uncoupling, preventing the |
| | formation of ATP. Used extensively against mites on fruit trees and formerly |
| | used as an antifouling agent and molluscacide; very toxic to aquatic life. |
| Pyrethroids | Acts by keeping open the sodium channels in neuronal membranes affecting |
| | both the peripheral and central nervous systems causing a hyper-excitable state. |
| | Symptoms include tremors, incoordination, hyperactivity and paralysis. |
| | Effective against most agricultural insect pests; extremely toxic to fish. |
| Nicotinoids | Act on the central nervous system causing irreversible blockage of the |
| | postsynaptic nicotinergic acetylcholine receptors. Used in the control of sucking |
| | insects, soil insects, whiteflies, termites, turf insects and the Colorado potato |
| | beetle. Generally have low toxicity to mammals, birds and fish. |
| Spinosyns | Acts by disrupting binding of acetylcholine in nicotinic acetylcholine receptors |
| | at the postsynaptic cell. Effective against caterpillars, lepidopteran larvae, leaf |
| | miners, thrips and termites. Regarded for its high level of specificity. |
| Pyrazoles | Inhibits mitochondrial electron transport at the NADH-CoQ reductase site |
| | leading to disruption of ATP formation. Effective against psylla, aphids, |
| | whitefly and thrips. Results of testing on one type (fipronil) indicate no effects |
| | on the clams, oysters or fish, with marginal effects on shrimp. |
| Pyridazinones | Interrupt mitochondrial electron transport at Site 1; mainly used as a miticide; |
| | display toxicity to aquatic arthropods and fish. |
| Quinazolines | Acts on the larval stages of most insect by inhibiting or blocking the synthesis |
| | of chitin in the exoskeleton. Developing larvae exhibit rupture of the malformed |
| | cuticle or death by starvation; not registered in U.S. |
| Botanicals | Depending upon the type can have various effects: |
| | Pyrethrum - affects both the central and peripheral nervous systems, |
| | stimulating nerve cells to produce repetitive discharges and eventually leading |
| | to paralysis. Commonly used to control lice. |
| | Nicotine - mimics acetylcholine (Ach) in the central nervous system ganglia, |
| | causing twitching, convulsions and death. Used most to control aphids and |
| | caterpillars. |
| | Rotenone - acts as a respiratory enzyme inhibitor. Used as a piscicide that kills |
| | all fish at doses non-toxic to fish food organisms. |
| | Limonene - affects the sensory nerves of the peripheral nervous system. Used |
| | to control fleas, lice, mites and ticks, while remaining virtually non-toxic to |
| | warm-blooded animals and only slightly toxic to fish. |
| | Neem - reduces feeding and disrupts molting by inhibiting biosynthesis or |
| | metabolism of ecdysone, the juvenile molting hormone. Commonly used |
| | against moth and butterfly larvae. |
| Synergists/ | Inhibit cytochrome P-450 dependent polysubstrate monooxygenases (PSMOs) |
| Activators | preventing the degradation of toxicants, enhancing the activity of insecticides |
| | when used in concert; synergists and activators are not in themselves considered |
| | toxic or insecticidal. |
| Antibiotics | Act by blocking the neurotransmitter GABA at the neuromuscular junction; |
| | feeding and egg laying stop shortly after exposure while death may take several |
| | days. Most promising use of these materials is the control of spider mites, |
| | leafminers and other difficult to control greenhouse pests. |
| Fumigants | Act as narcotics that lodge in lipid-containing tissues inducing narcosis, sleep or |
| | unconsciousness; pest affected depends on particular compound. |
| Inorganics | Mode of action is dependent upon type of inorganic: may uncouple oxidative |
| | phosphorylation (arsenicals), inhibit enzymes involved in energy production, or |
| | act as desiccants. Pest group depends on compound (e.g., sulfur for mites, boric |
| | acid for cockroaches). |
| Biorational | Grouped as biochemicals (hormones, enzymes, pheromones natural agents such |
| | as growth regulators) or microbials (viruses, bacteria, fungi, protozoa and |
| | nematodes). Act as either attractants, growth regulators or endotoxins; known |
| | for very low toxicity to non-target species. |
| Benzoylureas | Act as insect growth regulators by interfering with chitin synthesis. Greatest |
| | value is in the control of caterpillars and beetle larvae but is also registered for |
| | gypsy moth and mushroom fly. Some types are known for their impacts on |
| | invertebrates (reduced emergent species) and early life stages of sunfish |
| | (reduced weight) (Boyle et al. 1996). |

Reproduced from Insecticides, https://www.epa.gov/caddis-vol2/insecticides, last accessed on Apr. 29, 2023.

TABLE 2
Examples of Crops and Common Pesticides Used

| Crop | Pesticides |
|---|---|
| Corn, | Permethrin (pyrethroid), Esfenvalerate (pyrethroid), Bacillus thuringiensis (BT- |
| sweet | Biologicals), Diazinon (organophosphate), Methomyl (carbamate), Malathion |
| | (organophosphate), pyrethrin (botanical), Carbaryl (N-methyl carbamate), Endosulfan |
| | (organochlorine) |
| Alfalfa | Beta-cyfluthrin (pyrethyroid), Carbaryl (carbamate), Chlorpyrifos (organophosphate), |
| | Cyfluthrin (pyrethroid), Dimethoate (organophosphate), Gama-cyhalothrin (pyrethroid), |
| | Idoxacard (carboxylate), Methomyl (carbamate). Methyl Parathion (organophosphate), |
| | Permethrin (pyrethroid), Phosmet (organophosphate), Spinosad (fermentation product), |
| | Zeta-cypermethrin (pyrethroid) |
| Sorghum | Beta-cyfluthrin (pyrethyroid), Carbaryl (carbamate), Chlorpyrifos (organophosphate), |
| | Deltamethrin (pyrethroid), Dimethoate (organophosphate), Esfenvalerate (pyrethroid), |
| | Gama- and Lamda-cyhalothrin (pyrethroid), Malathion (organophosphate), Methidathion |
| | (organophosphate), Methomyl (cyclodine), Spinosad (fermentation product), Zeta- |
| | cypermethrin (pyrethroid) |
| Sunflower | Bacillus thuringiensis (bacterium), Beta-cyfluthrin (pyrethyroid), Carbaryl (carbamate), |
| | Chlorpyrifos (organophosphate), Deltamethrin (pyrethroid), Esfenvalerate (pyrethroid), |
| | Gama- and Lamda-cyhalothrin (pyrethroid), Methyl Parathion (organophosphate), Zeta- |
| | cypermethrin (pyrethroid) |
| Wheat | Beta-cyfluthrin (pyrethyroid), Carbaryl (carbamate), Chlorpyrifos (organophosphate), |
| | Dimethoate (organophosphate), Endosulfan (chlorinated hydrocarbon), Gama- and Lamda- |
| | cyhalothrin (pyrethroid), Malathion (organophosphate), Methidathion (organophosphate), |
| | Methomyl (cyclodine), Methyl parathion (organophosphate), Spinosad (fermentation |
| | product), Zeta-cypermethrin (pyrethroid) |
| Grapes | Sevin (carbaryl), Imidan (phosmet), Kelthane (dicofol), Guthion (azinphos methyl), |
| | Vendex (hexakis fenbutatin-oxide), Lanate (methomyl), Methoxychlor (methoxychlor), |
| | Provado (imidacloprid), Thiodan (endosulfan), Malathion, Neemix, Pyrethrins |
| Citrus | Cygon 400 (dimethoate), Cythion 57% (malathion), Diazinon AG500 (organophosphate), |
| | Dibrom 8E, Dipel 2X, Imidan 50 WP, Lannate L, Lorsban 15 G, Metasystox-R, Parathion |
| | 4E, Thiodan 3E, Zolone 3EC |
| Cotton | Acramite (bifenazate); Baythroid (cyfluthrin); Dimilin (diflubenzuron); Fulfill |
| | (pymetrozine); MSR (oxydemeton-methyl); Temik (aldicarb); Venom (dinotefuran); Zeal |
| | (etoxazole) |
| Soybeans | Asana XL (esfenvalerate); Baythroid 2 (cyfluthrin); Cruiser 5FS (thiamethoxam); |
| | Dimethoate 4E (organophosphate); Gaucho 480 (imidacloprid); Lorsban 4E (chlorpyrifos); |
| | Mustang Max (pyrethroid); Nufos 4E (chlorpyrifos); Warrior (organophosphate) |

Reproduced from Insecticides, https://www.epa.gov/caddis-vol2/insecticides, last accessed on Apr. 29, 2023.

When the application is to a plant, the nanoparticle, population, plurality, or compositions can be applied to the any part of the plant or its environs, e.g., the flower, roots, leaves, stems, branches, surrounding soil and the like.

A nanoparticle comprising an agent of the present disclosure can be administered for therapy by any suitable route of administration. It will also be appreciated that the optimal route will vary with the condition and age of the recipient, and the disease being treated.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample. In one aspect, the expression level of a gene from one sample may be directly compared to the expression level of that gene from a control or reference sample. In another aspect, the expression level of a gene from one sample may be directly compared to the expression level of that gene from the same sample following administration of a compound.

As used herein, “homology” or “identical”, percent “identity” or “similarity”, when used in the context of two or more nucleic acids or polypeptide sequences, refers to two or more sequences or subsequences that are the same or have a specified percentage of nucleotides or amino acid residues that are the same, e.g., at least 60% identity, preferably at least 65%, 70%, 75%, 80%, 85%, 90%, 91%, 92%, 93%, 94%, 95%, 96%, 97%, 98%, 99%, or higher identity over a specified region (e.g., nucleotide sequence encoding the chimeric PVX described herein). Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST. The terms “homology” or “identical,” percent “identity” or “similarity” also refer to, or can be applied to, the complement of a test sequence. The terms also include sequences that have deletions and/or additions, as well as those that have substitutions. As described herein, the preferred algorithms can account for gaps and the like. Preferably, identity exists over a region that is at least about 25 amino acids or nucleotides in length, or more preferably over a region that is at least 50-100 amino acids or nucleotides in length. An “unrelated” or “non-homologous” sequence shares less than 40% identity, or alternatively less than 25% identity, with one of the sequences disclosed herein.

It is to be inferred without explicit recitation and unless otherwise intended, that when the present disclosure relates to a polypeptide, protein, polynucleotide, an equivalent or a biologically equivalent of such is intended within the scope of this disclosure. As used herein, the term “biological equivalent thereof” is intended to be synonymous with “equivalent thereof” when referring to a reference protein, polypeptide or nucleic acid, intends those having minimal homology while still maintaining desired structure or functionality. Unless specifically recited herein, it is contemplated that any of the above also includes equivalents thereof. For example, an equivalent intends at least about 70% homology or identity, or at least 80% homology or identity and alternatively, or at least about 85%, or alternatively at least about 90%, or alternatively at least about 95%, or alternatively at least 98% percent homology or identity and/or exhibits substantially equivalent biological activity to the reference protein, polypeptide, or nucleic acid. Alternatively, when referring to polynucleotides, an equivalent thereof is a polynucleotide that hybridizes under stringent conditions to the reference polynucleotide or its complement.

The phrase “equivalent polypeptide” or “equivalent peptide fragment” refers to protein, polynucleotide, or peptide fragment encoded by a polynucleotide that hybridizes to a polynucleotide encoding the exemplified polypeptide or its complement of the polynucleotide encoding the exemplified polypeptide, under high stringency and/or which exhibit similar biological activity in vivo, e.g., approximately 100%, or alternatively, over 90% or alternatively over 85% or alternatively over 70%, as compared to the standard or control biological activity. Additional embodiments within the scope of this disclosure are identified by having more than 60%, or alternatively, more than 65%, or alternatively, more than 70%, or alternatively, more than 75%, or alternatively, more than 80%, or alternatively, more than 85%, or alternatively, more than 90%, or alternatively, more than 95%, or alternatively more than 97%, or alternatively, more than 98% or 99% sequence homology. Percentage homology can be determined by sequence comparison using programs such as BLAST run under appropriate conditions. In one aspect, the program is run under default parameters.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. A high stringency hybridization refers to a condition in which hybridization of an oligonucleotide to a target sequence comprises no mismatches (or perfect complementarity). Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

The term “isolated” as used herein refers to molecules or biologicals or cellular materials being substantially free from other materials. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

The term “protein”, “peptide” and “polypeptide” are used interchangeably and in their broadest sense to refer to a compound of two or more subunit amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any aspect of this technology that is a polynucleotide encompasses both the double-stranded form and each of two complementary single-stranded forms known or predicted to make up the double-stranded form.

As used herein, the term “purified” does not require absolute purity; rather, it is intended as a relative term. Thus, for example, a purified nucleic acid, peptide, protein, biological complexes or other active compound is one that is isolated in whole or in part from proteins or other contaminants. Generally, substantially purified peptides, proteins, biological complexes, or other active compounds for use within the disclosure comprise more than 80% of all macromolecular species present in a preparation prior to admixture or formulation of the peptide, protein, biological complex or other active compound with a pharmaceutical carrier, excipient, buffer, absorption enhancing agent, stabilizer, preservative, adjuvant or other co-ingredient in a complete pharmaceutical formulation for therapeutic administration. More typically, the peptide, protein, biological complex or other active compound is purified to represent greater than 90%, often greater than 95% of all macromolecular species present in a purified preparation prior to admixture with other formulation ingredients. In other cases, the purified preparation may be essentially homogeneous, wherein other macromolecular species are not detectable by conventional techniques.

The term “subject,” “host,” “individual,” and “patient” are as used interchangeably herein to refer to animals, typically mammalian animals. Any suitable mammal can be treated by a method, cell or composition described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. A mammal can be a pregnant female. In some embodiments a subject is a human. In some embodiments, a subject has or is suspected of having a cancer or neoplastic disorder.

As used herein, the term “nanoparticle” refers to particles that are on the order of 10-9 or one billionth of a meter and below 10-6 or 1 millionth of a meter in size. The term “nanoparticle” includes nanospheres; nanorods; nanoshells; and nanoprisms; and these nanoparticles may be part of a nanonetwork. The term “nanoparticles” also encompasses liposomes and lipid particles having the size of a nanoparticle. The particles may be, e.g., monodisperse or polydisperse and the variation in diameter of the particles of a given dispersion may vary, e.g., particle diameters of between about 0.1 to 100's of nm.

In some embodiments, the nanoparticle is of size about 1 nm to about 1000 nm, about 50 nm to about 500 nm, about 100 nm to about 250 nm, or about 200 nm to about 350 nm. In one embodiment, the nanoparticle is of about 100 nm to about 1000 nm. In another embodiment, the nanoparticle is of size about 80 nm to about 200 nm. In one embodiment, nanoparticle is of size about 50 nm to about 500 nm. In some embodiments, nanoparticle is of size about 158 nm, about 218 nm, or about 305 nm. In some embodiments, nanoparticle is of size about 337 nm, about 526 nm, about 569 nm, about 362 nm, about 476 nm, about 480 nm, about 676 nm, about 445 nm, about 434 nm, about 462 nm, about 492 nm, about 788 nm, about 463 nm, or about 65 nm.

The term “introduce” as applied to methods of producing modified cells refers to the process whereby a foreign (i.e. extrinsic or extracellular) agent is introduced into a host cell thereby producing a cell comprising the foreign agent. Methods of introducing nucleic acids include but are not limited to transduction, retroviral gene transfer, transfection, electroporation, transformation, viral infection, and other recombinant DNA techniques known in the art. In some embodiments, transduction is done via a vector (e.g., a viral vector). In some embodiments, transfection is done via a chemical carrier, DNA/liposome complex, or micelle (e.g., Lipofectamine (Invitrogen)). In some embodiments, viral infection is done via infecting the cells with a viral particle comprising the polynucleotide of interest.

The term “encapsulate” intends to entrap agents with the nanoparticle capsid and includes conjugation and non-conjugated interaction between the agent and nanoparticle capsid. Methods to encapsulate agents within nanoparticles are known in the art, e.g., as described in Caparco et al. (2023) “Delivery of Nematicides Using TMGMV-Derived Spherical Nanoparticles, Nano Lett., Vol. 28; 23 (12): 5785-5793; Chung et al. (2020) “Viral nanoparticles for drug delivery, imaging, immunotherapy, and theranostic applications” Adv. Drug Deliv. Rev. 2020; 156:214-235. doi: 10.1016/j.addr.2020.06.024. Epub 2020 Jun. 27. PMID: 32603813; PMCID: PMC7320870; and McNeale et al. (2022) “Protein cargo encapsulation by virus-like particles Strategies and applications” Wires, Nanomedicine and Nanobiotechnology, May/June 2023, available at wires.onlinelibrary.wiley.com/doi/10.1002/wnan 1869. To “encapsulate” small molecules, the molecules can be infused through the pores of the nanoparticle or encapsulated by disassembly into the capsid protein and reassembly of the capsid protein. For encapsulation of RNA molecules, because the native cargo of the virus-like particle is RNA, the capsid protein has an affinity to RNA such that these molecules also can be infused or encapsulated into the nanoparticle.

A “pharmaceutical composition” is intended to include the combination of an active agent with a carrier, inert or active, making the composition suitable for diagnostic or therapeutic use in vitro, in vivo or ex vivo.

“Pharmaceutically acceptable carriers” refers to any diluents, excipients, or carriers that may be used in the compositions disclosed herein. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They may be selected with respect to the intended form of administration, that is, oral tablets, capsules, elixirs, syrups and the like, and consistent with conventional pharmaceutical practices.

As used herein, the term “overexpress” with respect to a cell, a tissue, or an organ expresses a protein to an amount that is greater than the amount that is produced in a control cell, a control issue, or an organ. A protein that is overexpressed may be endogenous to the host cell or exogenous to the host cell.

The term “contacting” means direct or indirect binding or interaction between two or more. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

The term “introduce” as applied to methods of producing modified cells such as chimeric antigen receptor cells refers to the process whereby a foreign (i.e. extrinsic or extracellular) agent is introduced into a host cell thereby producing a cell comprising the foreign agent. Methods of introducing nucleic acids include but are not limited to transduction, retroviral gene transfer, transfection, electroporation, transformation, viral infection, and other recombinant DNA techniques known in the art. In some embodiments, transduction is done via a vector (e.g., a viral vector). In some embodiments, transfection is done via a chemical carrier, DNA/liposome complex, or micelle (e.g., Lipofectamine (Invitrogen)). In some embodiments, viral infection is done via infecting the cells with a viral particle comprising the polynucleotide of interest (e.g., AAV). In some embodiments, introduction further comprises CRISPR mediated gene editing or Transcription activator-like effector nuclease (TALEN) mediated gene editing. Methods of introducing non-nucleic acid foreign agents (e.g., soluble factors, cytokines, proteins, peptides, enzymes, growth factors, signaling molecules, small molecule inhibitors) include but are not limited to culturing the cells in the presence of the foreign agent, contacting the cells with the agent, contacting the cells with a composition comprising the agent and an excipient, and contacting the cells with vesicles or viral particles comprising the agent.

The term “culturing” refers to growing cells in a culture medium under conditions that favor expansion and proliferation of the cell. The term “culture medium” or “medium” is recognized in the art and refers generally to any substance or preparation used for the cultivation of living cells. The term “medium”, as used in reference to a cell culture, includes the components of the environment surrounding the cells. Media may be solid, liquid, gaseous or a mixture of phases and materials. Media include liquid growth media as well as liquid media that do not sustain cell growth. Media also include gelatinous media such as agar, agarose, gelatin and collagen matrices. Exemplary gaseous media include the gaseous phase to which cells growing on a petri dish or other solid or semisolid support are exposed. The term “medium” also refers to material that is intended for use in a cell culture, even if it has not yet been contacted with cells. In other words, a nutrient rich liquid prepared for culture is a medium. Similarly, a powder mixture that when mixed with water or other liquid becomes suitable for cell culture may be termed a “powdered medium.” “Defined medium” refers to media that are made of chemically defined (usually purified) components. “Defined media” do not contain poorly characterized biological extracts such as yeast extract and beef broth. “Rich medium” includes media that are designed to support growth of most or all viable forms of a particular species. Rich media often include complex biological extracts. A “medium suitable for growth of a high-density culture” is any medium that allows a cell culture to reach an OD600 of 3 or greater when other conditions (such as temperature and oxygen transfer rate) permit such growth. The term “basal medium” refers to a medium which promotes the growth of many types of microorganisms which do not require any special nutrient supplements. Most basal media generally comprise of four basic chemical groups: amino acids, carbohydrates, inorganic salts, and vitamins. A basal medium generally serves as the basis for a more complex medium, to which supplements such as serum, buffers, growth factors, lipids, and the like are added. In one aspect, the growth medium may be a complex medium with the necessary growth factors to support the growth and expansion of the cells of the disclosure while maintaining their self-renewal capability. Examples of basal media include, but are not limited to, Eagles Basal Medium, Minimum Essential Medium, Dulbecco's Modified Eagle's Medium, Medium 199, Nutrient Mixtures Ham's F-10 and Ham's F-12, McCoy's 5A, Dulbecco's MEM/F-I 2, RPMI 1640, and Iscove's Modified Dulbecco's Medium (IMDM).

Cytoplasmic type citrus leprosis virus-like particle (CiLV-C) intends the member is the type member of the genus (′ilevirus, family Kitaviridae. Its bacilliform particles surround a positive-sense ssRNA genome in two segments, each featuring a 5′ cap and 3′ polyadenylate tail. The first segment (RNA1) contains two open reading frames (ORFs) encoding a multi-domain replication-associated protein and the capsid protein, p29. The second segment (RNA2) contains four ORFs encoding p15, which is required for the formation of vesicles in the ER, the p61 glycoprotein with roles in the remodeling of the ER and Golgi body, the movement protein p32, and the integral membrane protein p24, which is also involved in viral replication and assembly in the ER and may function as a matrix protein. The complete nucleotide sequence of the CiLV-C virus is known in the art and published for example in Pascon et al. (2006), “The complete nucleotide sequence and genomic organization of Citrus Leprosis Associated Virus, Cytoplasmic Type C (CiLV-C)” Virus Genes, Vol. 32 (3): 289-298. The p61 protein and encoding polynucleotide sequences are known in the art and the amino acid sequences is provided at NCBI Ref. No.: YP 654541.1, last accessed on Sep. 6, 2023. The p24 protein and encoding polynucleotide sequences are known in the art and the amino acid sequences is provided at NCBI Ref. No.: YP_654543.1, last accessed on Sep. 6, 2023.

TMV Omega (TMV (2) intends the translational enhancer from tobacco mosaic virus (TMV) 5′ leader sequence, as referenced in Gallie et al. (1988) “Mutational analysis of the tobacco mosaic virus 5′-leader for altered ability to enhance translation” Nucleic Acids Res. February 11; 16 (3): 883-893.

p29 coat protein of CiLV-C intends the capsid protein p29 is known in the art (see https://www.uniprot.org/uniprotkb/A0AON9EH07/entry, last accessed on Sep. 1, 2023) and equivalents thereof. A sequence of the gene encoding the CiLV-C p29 coat protein is provided in FIGS. 6B and 7B, (SEQ ID NO.: 1) and the p29 protein is provided in FIGS. 6B and 7B (SEQ ID NO: 2). An optimized polynucleotide encoding the p29_CLIVS polypeptide is provided in FIG. 7B and identified as p29_CLIVS (SEQ ID NO: 2).

### Modes for Carrying Out the Disclosure

### CiLV-C Nanoparticles

Applicant provides herein a Cytoplasmic type citrus leprosis virus-like particle (CiLV-C) nanoparticle comprising a cytoplasmic type citrus leprosis virus-like particle (CiLV-C) p29 protein coat and optionally an agent encapsulated with the p29 protein coat. In one aspect, the p29 coat protein of the nanoparticle comprises the polypeptide of SEQ ID NO: 2 (see FIG. 7B) or an equivalent thereof or is the polypeptide encoded by the polynucleotide of SEQ ID NO: 1 (see FIG. 7B) or its equivalent. In another aspect, the nanoparticle lacks the CiLV-C p61 glycoprotein or its equivalent and the CiL V-C integral membrane protein p24 or its equivalent. In another aspect, the polynucleotide encoding the CiLV-C virus-like nanoparticle further comprises a the CiLV-C movement protein p32 or its equivalent or the tobacco mosaic virus (TMV) movement protein (MP) or its equivalent, optionally encoded by the polynucleotide of SEQ ID NO: 5, e.g., encoding the TMV MP of SEQ ID NO: 6, which is shown in FIG. 7B. The nanoparticle can be detectably labeled and optionally comprise an agent as described herein. Also provided are polynucleotides encoding the nanoparticles, that are optionally detectably labeled.

In one aspect, the p29 coat protein of the nanoparticle comprises the polypeptide of SEQ ID NO: 2 (see FIG. 7B) or its equivalent, or is encoded by a polynucleotide of SEQ ID NO: 1 (see FIG. 7B) or its equivalent. In another aspect, the CiLV-C nanoparticle lacks the CiLV-C p61 glycoprotein or its equivalent and the CiLV-C integral membrane protein p24 or its equivalent. In another aspect, CiLV-C virus-like nanoparticle further comprises the CiLV-C movement protein p32 or its equivalent or a tobacco mosaic virus (TMV) movement protein (MP) or its equivalent, optionally the TMV MP of SEQ ID NO: 6, which is shown in FIG. 7B. In one aspect, the CiLV-C nanoparticle is the polypeptide shown in FIG. 7B (SEQ ID NO: 8) or is the polypeptide encoded by the polypeptide shown in FIG. 7B (SEQ ID NO: 7). The nanoparticle can be detectably labeled. Also provided are polynucleotides encoding the nanoparticles, that are optionally detectably labeled.

Further provided are a plurality and/or a population of nanoparticles, wherein the nanoparticles can be the same or different from each other with respect to the optional agent and/or the composition of the nanoparticle and/or the detectable label.

Applicant also provides herein a Cytoplasmic type citrus leprosis virus-like particle (CiLV-C) nanoparticle comprising a cytoplasmic type citrus leprosis virus-like particle (CiLV-C) p29 protein coat and a therapeutic agent encapsulated with the p29 protein coat. In one aspect, the p29 coat protein of the nanoparticle comprises the polypeptide of SEQ ID NO: 2 (see FIG. 7B) or its equivalent or is encoded by a polynucleotide of SEQ ID NO: 1 (see FIG. 7B) or its equivalent. In another aspect, the CiLV-C nanoparticle lacks the CiLV-C p61 glycoprotein or its equivalent and the CiLV-C integral membrane protein p24 or its equivalent. In another aspect, CiLV-C virus-like nanoparticle further comprises the CiLV-C movement protein p32 or its equivalent or a tobacco mosaic virus (TMV) movement protein (MP) or its equivalent, optionally the TMV MP of SEQ ID NO: 6 or its equivalent, which is shown in FIG. 7B. In one aspect, the CiLV-C nanoparticle is the polypeptide shown in FIG. 7B (SEQ ID NO: 8) or its equivalent or is the polypeptide encoded by the polypeptide shown in FIG. 7B (SEQ ID NO: 7) or its equivalent.

Non-limiting examples of therapeutic agents include for example a small molecule, a polypeptide or a polynucleotide, optionally an RNA or a DNA molecule such as a therapeutic RNA vaccine. Additional non-limiting examples of such include for example, a chemotherapeutic agent, an immunotherapeutic agent, a targeted therapy, radiation therapy, or a combination thereof. For the treatment of cancer, illustrative additional therapeutic agents include, but are not limited to, alkylating agents such as altretamine, busulfan, carboplatin, carmustine, chlorambucil, cisplatin, cyclophosphamide, dacarbazine, lomustine, melphalan, oxalaplatin, temozolomide, or thiotepa; antimetabolites such as 5-fluorouracil (5-FU), 6-mercaptopurine (6-MP), capecitabine, cytarabine, floxuridine, fludarabine, gemcitabine, hydroxyurea, methotrexate, or pemetrexed; anthracyclines such as daunorubicin, doxorubicin, epirubicin, or idarubicin; topoisomerase I inhibitors such as topotecan or irinotecan (CPT-11); topoisomerase II inhibitors such as etoposide (VP-16), teniposide, or mitoxantrone; mitotic inhibitors such as docetaxel, estramustine, ixabepilone, paclitaxel, vinblastine, vincristine, or vinorelbine; or corticosteroids such as prednisone, methylprednisolone, or dexamethasone.

In another aspect, the therapeutic agent is a polynucleotide, e.g. a DNA or a RNA molecule, e.g. antisense RNA, or a therapeutic RNA molecule.

In another aspect, the agricultural agent is a nematicide selected from the group of Ivermectin, fluazaindolizine, fluensulfone, or fluopyram. In another aspect, the agricultural agent is an RNAi inducer such as an RNAi molecule such as small interfering RNAs (siRNA) that target nematodes, viruses, insects and the like.

Further provided are a plurality and/or a population of nanoparticles, wherein the nanoparticles can be the same or different from each other with respect to the therapeutic agent and/or composition of the nanoparticle, and/or the detectable label.

Applicant also provides herein a Cytoplasmic type citrus leprosis virus-like particle (CiLV-C) nanoparticle comprising a cytoplasmic type citrus leprosis virus-like particle (CiLV-C) p29 protein coat or its equivalent and an agricultural agent encapsulated with the p29 protein coat or its equivalent. In one aspect, the p29 coat protein of the nanoparticle comprises the polypeptide of SEQ ID NO: 2 (see FIG. 7B) or its equivalent or is encoded by a polynucleotide of SEQ ID NO: 1 (see FIG. 7B) or its equivalent. In another aspect, the CiLV-C nanoparticle lacks the CiLV-C p61 glycoprotein or its equivalent and the CiLV-C integral membrane protein p24 or its equivalent. In another aspect, CiLV-C virus-like nanoparticle further comprises the CiLV-C movement protein p32 or its equivalent or a tobacco mosaic virus (TMV) movement protein (MP) or its equivalent, optionally the TMV MP of SEQ ID NO: 6 or its equivalent, which is shown in FIG. 7B. In one aspect, the CiLV-C nanoparticle is the polypeptide shown in FIG. 7B (SEQ ID NO: 8) or its equivalent or is the polypeptide encoded by the polypeptide shown in FIG. 7B (SEQ ID NO: 7) or its equivalent. Non-limiting examples of an agricultural agent such as a pesticide or an insecticide, examples of such are provided in Table 2. The nanoparticle can be detectably labeled.

Further provided are a plurality and/or a population of nanoparticles, wherein the nanoparticles can be the same or different from each other with respect to the agricultural agent and/or composition of the nanoparticle and/or the detectable label.

### CiLV-C Nanoparticle Polynucleotides, Vectors and Host Cells

Applicant further provides herein a polynucleotide encoding a Cytoplasmic type citrus leprosis virus-like particle (CiLV-C) nanoparticle comprising a cytoplasmic type citrus leprosis virus-like particle (CiLV-C) p29 protein coat or its equivalent. In one aspect, the p29 coat protein of the nanoparticle comprises the polynucleotide encoding the polypeptide of SEQ ID NO: 2 (see FIG. 7B) or its equivalent or is the polynucleotide of SEQ ID NO: 1 (see FIG. 7B) or its equivalent. In another aspect, the polynucleotide encoding the CiLV-C nanoparticle lacks the polynucleotide encoding the CiLV-C p61 glycoprotein or its equivalent and the polynucleotide encoding the CiLV-C integral membrane protein p24 or its equivalent. In another aspect, the polynucleotide encoding the CiLV-C virus-like nanoparticle further comprises a polynucleotide encoding the CiLV-C movement protein p32 or its equivalent or a polynucleotide encoding tobacco mosaic virus (TMV) movement protein (MP) or its equivalent, optionally the polynucleotide of SEQ ID NO: 5 or its equivalent, e.g., encoding the TMV MP of SEQ ID NO: 6 or its equivalent, which is shown in FIG. 7B. In a further aspect, provided herein is a recombinant polynucleotide comprising: an expression vector; a TMV replicase (SEQ ID NO: 3) or its equivalent; a polynucleotide encoding a CiLV-C p29 protein (SEQ ID NO: 1) or its equivalent, a movement protein p32 or a TMV movement protein (SEQ ID NO: 5) or its equivalent. In one aspect, the recombinant polynucleotide comprises, or consists essentially of, or yet further consists of the polynucleotide shown in FIG. 7B (SEQ ID NO: 7). Further provided is the polypeptide encoded by the recombinant polynucleotide. The polynucleotide can be a DNA molecule or a RNA molecule, that is optionally detectably labeled.

Further provided are a plurality and/or a population of polynucleotides as provided herein, wherein the polynucleotides can be the same or different from each other with respect to the polynucleotide sequences and/or the detectable label.

Also provided are vectors comprising the polynucleotides, such as plasmids or viral vectors. Non-limiting examples of vectors include the plant expression vector pUC57, a sequence of which is available at and commercially available from Genscript (www.genscript.com/vector/SD1176-pUC57_plasmid_DNA.html, last accessed on Sep. 2, 2023) or Thermofisher (see www.thermofisher.com/order/catalog/product/SD0171, last accessed on Sep. 2, 2023) or pTRBO (tobacco mosaic virus RNA-based overexpression, and commercially available from Addgene, www.addgene.org/80083/, last accessed on Sep. 2, 2023).

Also provided are host cells comprising the polynucleotides and polypeptides as described herein. The host cells can be prokaryotic or eukaryotic cells. In one aspect, the cell is a plant cell that is used for propagation of the CiLV-C nanoparticle, e.g., Agrobacterium tumefaciens, e.g. strain GV3101 (Gold Biotechnology), Brevipalpus spp., or N. benthamiana plant cells, e.g. leaves.

Further provided are a plurality and/or a population of vectors and/or host cells as described herein, wherein the vectors and/or host cells, and/or polynucleotides, and/or detectable labels can be the same or different from each other.

### Compositions

Also provided are compositions comprising one or more of: the CiLV-C nanoparticle, the polynucleotides, vectors, host cells, or plurality of each of same and a carrier, optionally a pharmaceutical or an agriculturally appropriate carrier, e.g., water or other carrier.

In one aspect, the composition further comprises a preservative or stabilizer, that can be in one aspect, exogenously added or non-naturally occurring. In another aspect, the composition is lyophilized or freeze-dried for ease of transport.

In another embodiment, this technology relates to a pharmaceutical or an agricultural composition as described herein and a pharmaceutically or agriculturally acceptable carrier.

Compositions, including pharmaceutical or agricultural compositions comprising, consisting essentially of, or consisting of the nanoparticle alone or in combination of other therapeutic or agricultural agents can be manufactured by means of conventional mixing, dissolving, granulating, dragee-making levigating, emulsifying, encapsulating, entrapping, or lyophilization processes. These can be formulated in conventional manner using one or more physiologically acceptable carriers, diluents, excipients, or auxiliaries which facilitate processing of the combinations of compounds provided herein into preparations which can be used pharmaceutically.

In some embodiments, the compositions described herein are administered to a subject by multiple administration routes, including but not limited to, parenteral, oral, buccal, rectal, sublingual, or transdermal administration routes. In some cases, parenteral administration comprises, or consists essentially of, or yet further consists of, intravenous, subcutaneous, intramuscular, intracerebral, intranasal, intra-arterial, intra-articular, intradermal, intravitreal, intraosseous infusion, intraperitoneal, or intrathecal administration. In some instances, the pharmaceutical composition is formulated for local administration. In other instances, the pharmaceutical composition is formulated for systemic administration.

In some embodiments, the compositions include, but are not limited to, lyophilized compositions, aqueous liquid dispersions, self-emulsifying dispersions, solid solutions, liposomal dispersions, aerosols, solid dosage forms, powders, immediate release compositions, controlled release compositions, fast melt compositions, tablets, capsules, pills, delayed release compositions, extended release compositions, pulsatile release compositions, multiparticulate compositions (e.g., nanoparticle compositions), and mixed immediate and controlled release compositions.

In some embodiments, the compositions include a carrier or carrier materials selected on the basis of compatibility with the composition disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. Compatible carrier materials include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995), Hoover, John E., Remington's Pharmaceutical Sciences, Mack Publishing Co., Easton, Pennsylvania 1975, Liberman, H. A. and Lachman, L., Eds., Pharmaceutical Dosage Forms, Marcel Decker, New York, N.Y., 1980, and Pharmaceutical Dosage Forms and Drug Delivery Systems, Seventh Ed. (Lippincott Williams & Wilkins, 1999).

In some instances, the compositions further include pH adjusting agents or buffering agents which include acids such as acetic, boric, citric, lactic, phosphoric and hydrochloric acids, bases such as sodium hydroxide, sodium phosphate, sodium borate, sodium citrate, sodium acetate, sodium lactate and tris-hydroxymethylaminomethane, and buffers such as citrate/dextrose, sodium bicarbonate and ammonium chloride. Such acids, bases and buffers are included in an amount required to maintain pH of the composition in an acceptable range.

In some instances, the compositions include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions, suitable salts include sodium chloride, potassium chloride, sodium thiosulfate, sodium bisulfite and ammonium sulfate.

In some embodiments, the compositions include, but are not limited to, sugars like trehalose, sucrose, mannitol, maltose, glucose, or salts like potassium phosphate, sodium citrate, ammonium sulfate and/or other agents such as heparin to increase the solubility and in vivo stability of polypeptides.

In some instances, the compositions further include diluent which are used to stabilize nanoparticles because they can provide a more stable environment. Salts dissolved in buffered solutions (which also can provide pH control or maintenance) are utilized as diluents in the art, including, but not limited to a phosphate buffered saline solution. In certain instances, diluents increase bulk of the composition to facilitate compression or create sufficient bulk for homogenous blend for capsule filling. Such compounds can include e.g., lactose, starch, mannitol, sorbitol, dextrose, microcrystalline cellulose such as AVICEL®, dibasic calcium phosphate, dicalcium phosphate dihydrate, tricalcium phosphate, calcium phosphate, anhydrous lactose, spray-dried lactose, pregelatinized starch, compressible sugar, such as Di-PAC® (Amstar), mannitol, hydroxypropylmethylcellulose, hydroxypropylmethylcellulose acetate stearate, sucrose-based diluents, confectioner's sugar, monobasic calcium sulfate monohydrate, calcium sulfate dihydrate, calcium lactate trihydrate, dextrates, hydrolyzed cereal solids, amylose, powdered cellulose, calcium carbonate, glycine, kaolin, mannitol, sodium chloride, inositol, bentonite, and the like.

In some cases, the pharmaceutical compositions include disintegration agents or disintegrants to facilitate the breakup or disintegration of a substance. The term “disintegrate” include both the dissolution and dispersion of the dosage form when contacted with gastrointestinal fluid. Examples of disintegration agents include a starch, e.g., a natural starch such as corn starch or potato starch, a pregelatinized starch such as National 1551 or AMIJEL®, or sodium starch glycolate such as PROMOGEL® or EXPLOTAB®, a cellulose such as a wood product, methylcrystalline cellulose, e.g., AVICEL®, AVICEL® PH101, AVICEL® PH102, AVICEL® PH105, ELCEMA® P100, EMCOCEL®, VIVACEL®, MING TIA®, and SOLKA-FLOC®, methylcellulose, croscarmellose, or a cross-linked cellulose, such as cross-linked sodium carboxymethylcellulose (AC-DI-SOLR), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as VEEGUM® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

In some instances, the pharmaceutical compositions include filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

Lubricants and glidants are also optionally included in the pharmaceutical compositions described herein for preventing, reducing or inhibiting adhesion or friction of materials.

Exemplary lubricants include, e.g., stearic acid, calcium hydroxide, talc, sodium stearyl fumerate, a hydrocarbon such as mineral oil, or hydrogenated vegetable oil such as hydrogenated soybean oil (STEROTEX®), higher fatty acids and their alkali-metal and alkaline earth metal salts, such as aluminum, calcium, magnesium, zinc, stearic acid, sodium stearates, glycerol, talc, waxes, STEAROWET®, boric acid, sodium benzoate, sodium acetate, sodium chloride, leucine, a polyethylene glycol (e.g., PEG-4000) or a methoxypolyethylene glycol such as CARBOWAX™, sodium oleate, sodium benzoate, glyceryl behenate, polyethylene glycol, magnesium or sodium lauryl sulfate, colloidal silica such as SYLOID™, CAB-O-SIL®, a starch such as corn starch, silicone oil, a surfactant, and the like.

Plasticizers include compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. Plasticizers can also function as dispersing agents or wetting agents.

Solubilizers include compounds such as triacetin, triethyl citrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

Stabilizers include compounds such as any antioxidation agents, buffers, acids, preservatives and the like. Exemplary stabilizers include L-arginine hydrochloride, tromethamine, albumin (human), citric acid, benzyl alcohol, phenol, disodium biphosphate dehydrate, propylene glycol, metacresol or m-cresol, zinc acetate, poly sorb ate-20 or TWEEN® 20, or trometamol.

Suspending agents include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Surfactants include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., PLURONIC® (BASF), and the like. Additional surfactants include polyoxyethylene fatty acid glycerides and vegetable oils, e.g., polyoxyethylene (60) hydrogenated castor oil, and polyoxyethylene alkyl ethers and alkylphenyl ethers, e.g., octoxynol 10, octoxynol 40. Sometimes, surfactants is included to enhance physical stability or for other purposes.

Viscosity enhancing agents include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

Wetting agents include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

The pharmaceutical compositions for the administration of the combinations of compounds can be conveniently presented in dosage unit form and can be prepared by any of the methods well known in the art of pharmacy. The pharmaceutical compositions can be, for example, prepared by uniformly and intimately bringing the compounds provided herein into association with a liquid carrier, a finely divided solid carrier or both, and then, if necessary, shaping the product into the desired formulation. In the pharmaceutical composition, each compound of the combination provided herein is included in an amount sufficient to produce the desired therapeutic effect. For example, pharmaceutical compositions of the present technology may take a form suitable for virtually any mode of administration, including, for example, topical, ocular, oral, buccal, systemic, nasal, injection, infusion, transdermal, rectal, and vaginal, or a form suitable for administration by inhalation or insufflation.

For topical administration, the combination of compounds can be formulated as solutions, gels, ointments, creams, suspensions, etc., as is well-known in the art.

Systemic formulations include those designed for administration by injection (e.g., subcutaneous, intravenous, infusion, intramuscular, intrathecal, or intraperitoneal injection) as well as those designed for transdermal, transmucosal, oral, or pulmonary administration.

Useful injectable preparations include sterile suspensions, solutions, or emulsions of the compounds provided herein in aqueous or oily vehicles. The compositions may also contain formulating agents, such as suspending, stabilizing, and/or dispersing agents. The formulations for injection can be presented in unit dosage form, e.g., in ampules or in multidose containers, and may contain added preservatives.

Alternatively, the injectable formulation can be provided in powder form for reconstitution with a suitable vehicle, including but not limited to sterile pyrogen free water, buffer, and dextrose solution, before use. To this end, the combination of compounds provided herein can be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets, or capsules prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulfate). The tablets can be coated by methods well known in the art with, for example, sugars, films, or enteric coatings.

Compositions intended for oral use can be prepared according to any method known to the art for the manufacture of pharmaceutical compositions, and such compositions may contain one or more agents selected from the group consisting of sweetening agents, flavoring agents, coloring agents, and preserving agents in order to provide pharmaceutically elegant and palatable preparations. Tablets contain the combination of compounds provided herein in admixture with non-toxic pharmaceutically acceptable excipients which are suitable for the manufacture of tablets. These excipients can be for example, inert diluents, such as calcium carbonate, sodium carbonate, lactose, calcium phosphate or sodium phosphate; granulating and disintegrating agents (e.g., corn starch or alginic acid); binding agents (e.g. starch, gelatin, or acacia); and lubricating agents (e.g., magnesium stearate, stearic acid, or talc). The tablets can be left uncoated or they can be coated by known techniques to delay disintegration and absorption in the gastrointestinal tract and thereby provide a sustained action over a longer period. For example, a time delay material such as glyceryl monostearate or glyceryl distearate can be employed. They may also be coated by the techniques well known to the skilled artisan. The pharmaceutical compositions of the present technology may also be in the form of oil-in-water emulsions.

Liquid preparations for oral administration may take the form of, for example, elixirs, solutions, syrups, or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives, or hydrogenated edible fats); emulsifying agents (e.g., lecithin, or acacia); non-aqueous vehicles (e.g., almond oil, oily esters, ethyl alcohol, Cremophore™, or fractionated vegetable oils); and preservatives (e.g., methyl or propyl-p-hydroxybenzoates or sorbic acid). The preparations may also contain buffer salts, preservatives, flavoring, coloring, and sweetening agents as appropriate.

### Kits

In some embodiments, one or more compositions disclosed herein are contained in a kit. Accordingly, in some embodiments, provided herein is a kit comprising, consisting essentially of, or consisting of one or more compositions disclosed herein and instructions for their use.

In one aspect, the present disclosure provides kits for performing the methods of this disclosure as well as instructions for carrying out the methods of the present disclosure. The kit comprises, or alternatively consists essentially of, or yet further consists of one or more of the nanoparticles, or the plurality of the composition, of this disclosure and instructions for use. In a further aspect, the instruction for use provides directions to conduct any of the methods disclosed herein.

The kit components, (e.g., reagents) can be packaged in a suitable container. The kit can also comprise, or alternatively consist essentially of, or yet further consist of, e.g., a buffering agent, a preservative or a protein-stabilizing agent. Each component of the kit can be enclosed within an individual container and all of the various containers can be within a single package, along with instructions for interpreting the results of the assays performed using the kit. The kits of the present disclosure may contain a written product on or in the kit container. The written product describes how to use the reagents contained in the kit.

As amenable, these suggested kit components may be packaged in a manner customary for use by those of skill in the art. For example, these suggested kit components may be provided in solution or as a liquid dispersion or the like.

As is apparent to those of skill in the art, the aforementioned methods and compositions can be combined with other therapeutic composition and agents for the treatment or the disclosed diseases or conditions.

### Methods

Also provided herein is a CiLV-C virus nanoparticle produced by expressing the polynucleotide as described herein in an organism infected plant cell. In one aspect, the organism comprises A. tumefaciens and the plant cell comprises N. benthamiana. In one aspect, the nanoparticle comprises a Cytoplasmic type citrus leprosis virus-like particle (CiLV-C) nanoparticle comprising a cytoplasmic type citrus leprosis virus-like particle (CiLV-C) p29 protein coat or its equivalent. In one aspect, the p29 coat protein of the nanoparticle comprises the polypeptide of SEQ ID NO: 2 or its equivalent (see FIG. 7B) or is the polypeptide encoded by the polynucleotide of SEQ ID NO: 1 or its equivalent (see FIG. 7B). In another aspect, the nanoparticle lacks the CiLV-C p61 glycoprotein or its equivalent and the CiLV-C integral membrane protein p24 or its equivalent. In another aspect, the polynucleotide encoding the CiLV-C virus-like nanoparticle further comprises a the CIL V-C movement protein p32 or its equivalent or the tobacco mosaic virus (TMV) movement protein (MP) or its equivalent, optionally encoded by the polynucleotide of SEQ ID NO: 5 or its equivalent, e.g., encoding the TMV MP of SEQ ID NO: 6 or its equivalent, which is shown in FIG. 7B. The nanoparticle can be detectably labeled and optionally comprise an agent as described herein.

Further provided are a plurality and/or a population of nanoparticles wherein the nanoparticles can be the same or different from each other with respect to the polypeptides and/or the detectable label.

Applicant provides herein a method to deliver a CiLV-C nanoparticle to a cell, wherein the CiLV-C nanoparticle optionally comprises an agent (e.g., therapeutic or an agricultural agent), comprising contacting the cell with the CiLV-C virus-like particle as described above. In one aspect, the nanoparticle comprises a Cytoplasmic type citrus leprosis virus-like particle (CiLV-C) nanoparticle comprising a cytoplasmic type citrus leprosis virus-like particle (CiLV-C) p29 protein coat. In one aspect, the p29 coat protein or its equivalent of the nanoparticle comprises the polypeptide of SEQ ID NO: 2 or its equivalent (see FIG. 7B) or is the polypeptide encoded by the polynucleotide of SEQ ID NO: 1 or its equivalent (see FIG. 7B). In another aspect, the nanoparticle lacks the CiLV-C p61 glycoprotein or its equivalent and the CiLV-C integral membrane protein p24 or its equivalent. In another aspect, the polynucleotide encoding the CiLV-C virus-like nanoparticle further comprises a the CiLV-C movement protein p32 or its equivalent or the tobacco mosaic virus (TMV) movement protein (MP) or its equivalent, optionally encoded by the polynucleotide of SEQ ID NO: 5 or its equivalent, e.g., encoding the TMV MP of SEQ ID NO: 6 or its equivalent, which is shown in FIG. 7B. The nanoparticle can be detectably labeled and optionally comprise an agent as described herein.

In one aspect, the cell is a cell of a plant (e.g., roots, stem, leaves, flowers, buds, etc.) a prokaryotic cell (e.g., a bacteria or virus), or a eukaryotic cell (e.g., an animal cell, such as a mammalian, a human, a canine, a bovine, a feline, an equine, or a porcine for example. The contacting can be in vitro, ex vivo or in vivo. The contacting can be by spraying or applying the CiLV-C nanoparticle to an agricultural product or by contacting the cell with the nanoparticle. Additionally, the nanoparticle can be administered in vivo to a subject in need thereof.

Accordingly, this disclosure also provides a method to deliver a CiLV-C nanoparticle, or when the nanoparticle comprises an agent, to a subject in need thereof, comprising administering to a subject the CiLV-C virus-like particle as described herein. Non-limiting examples of subjects include an animal, a mammal, a human, a canine, a feline, an equine, a porcine, a simian, a murine or a rat. In one aspect, the nanoparticle comprises a Cytoplasmic type citrus leprosis virus-like particle (CiLV-C) nanoparticle comprising a cytoplasmic type citrus leprosis virus-like particle (CiLV-C) p29 protein coat or its equivalent. In one aspect, the p29 coat protein of the nanoparticle comprises the polypeptide of SEQ ID NO: 2 or its equivalent (see FIG. 7B) or is the polypeptide encoded by the polynucleotide of SEQ ID NO: 1 or its equivalent (see FIG. 7B). In another aspect, the nanoparticle lacks the CiLV-C p61 glycoprotein or its equivalent and the CiLV-C integral membrane protein p24 or its equivalent. In another aspect, the polynucleotide encoding the CiLV-C virus-like nanoparticle further comprises a the CiLV-C movement protein p32 or its equivalent or the tobacco mosaic virus (TMV) movement protein (MP), optionally encoded by the polynucleotide of SEQ ID NO: 5 or its equivalent, e.g., encoding the TMV MP of SEQ ID NO: 6 or its equivalent, which is shown in FIG. 7B. The nanoparticle can be detectably labeled and optionally comprise an agent as described herein.

Further provided is a method to package an agent comprising contacting the CiLV-C virus nanoparticle as described herein with the agent, e.g. a therapeutic agent or an agricultural agent. In one aspect, the therapeutic agent comprises a polynucleotide, e.g., DNA or RNA. In another aspect, the agent is an agricultural agent such as a pesticide or an insecticide, see, e.g., Table 2. In a further aspect, the method further comprises isolating the CiLV-C virus particle from the plant cell. In one aspect, the nanoparticle comprises a Cytoplasmic type citrus leprosis virus-like particle (CiLV-C) nanoparticle comprising a cytoplasmic type citrus leprosis virus-like particle (CiLV-C) p29 protein coat or its equivalent. In one aspect, the p29 coat protein of the nanoparticle comprises the polypeptide of SEQ ID NO: 2 or its equivalent (see FIG. 7B) or is the polypeptide encoded by the polynucleotide of SEQ ID NO: 1 or its equivalent (see FIG. 7B). In another aspect, the nanoparticle lacks the CiLV-C p61 glycoprotein or its equivalent and the CiLV-C integral membrane protein p24 or its equivalent. In another aspect, the polynucleotide encoding the CiLV-C virus-like nanoparticle further comprises a the CiLV-C movement protein p32 or its equivalent or the tobacco mosaic virus (TMV) movement protein (MP) or its equivalent, optionally encoded by the polynucleotide of SEQ ID NO: 5, or its equivalent e.g., encoding the TMV MP of SEQ ID NO: 6 or its equivalent, which is shown in FIG. 7B. The nanoparticle can be detectably labeled and optionally comprise an agent as described herein.

### Experiment No. 1

Many plant virus-like particles (VLPs) utilized in nanotechnology are 30-nm icosahedrons. To expand the VLP platforms, Applicant produced VLPs of Cytoplasmic type citrus leprosis virus (CiLV-C) in Nicotiana benthamiana. Applicant were interested in CiLV-C because of its unique bacilliform shape (60-70 nm×110-120 nm). The CiLV-C capsid protein (p29) gene was transferred to the pTRBO expression vector transiently expressed in leaves. Stable VLPs were formed, as confirmed by agarose gel electrophoresis, transmission electron microscopy and size exclusion chromatography. Interestingly, the morphology of the VLPs (15.8±1.3 nm icosahedral particles) differed from that of the native bacilliform particles indicating that the assembly of native virions is influenced by other viral proteins and/or the packaged viral genome. The smaller CiLV-C VLPs will also be useful for structure-function studies to compare with the 30-nm icosahedrons of other VLPs.

To learn more about the assembly of CiLV-C, Applicant transiently expressed the p29 capsid protein in Nicotiana benthamiana plants in an attempt to generate virus-like particles (VLPs). Unlike native viruses, VLPs lack any genomic material and are therefore unable to replicate, but they are often structurally similar to the parental virus and still capable of interacting with target cells [20]. Therefore, CiLV-C VLPs would make a useful addition to the viral nanoparticle platforms currently being investigated. The availability of VLPs would also allow us to work on the development of more effective and less environmentally hazardous control measures to prevent citrus leprosis.

### Methods

### Expression and Purification of CiLV-(VLPs

The wild-type CiLV-C p29 sequence (UniProt: Q1KZ58) was reverse translated and codon optimized for N. benthamiana using SnapGene. The cDNA was synthesized by Genscript Biotech and transferred from the source vector pUC57-mini_P29_CiL VC to the PacI-AvrII site of vector pTRBO [21], a gift from John Lindbo (Addgene #80082). The insert was verified by colony PCR using primers TRBO-f (5′-GAT GAT TCG GAG GCT ACT GTC-3′) and P29-r 5′-CAG AAG GAC CAG GTT GAA GTT G-3′). The reaction was heated to 95° C. for 30 s followed by 30 cycles of 95° C. for 30 s, 60° C. for 30 s and 68° C. for 30 s, and a final elongation step at 68° C. for 5 min. The presence of the insert was confirmed by digestion with the restriction enzymes PacI and AvrII (New England Biolabs) followed by 1% agarose gel electrophoresis. Agrobacterium tumefaciens strain GV3101 (Gold Biotechnology) was transformed by electroporation and cultured at 28° C. in YEB medium (5 g/L beef extract, 1 g/L yeast extract, 5 g/L peptone, 5 g/L sucrose, 0.5 g/L MgCl2) supplemented with 50 μg/ml kanamycin, 30 μg/ml gentamycin and 25 μg/ml rifampicin. Bacterial cultures were resuspended in infiltration buffer (10 mM MES pH 5.5, 10 mM MgCl₂, 2% sucrose, 200 μM acetosyringone) and the OD_{600 nm} was adjusted to 1. The suspension was incubated for 4 h at room temperature before adding 0.01% (v/v) Silwet L-77 immediately prior to vacuum infiltration. Leaves of 6-week-old N. benthamiana plants were vacuum infiltrated as previously described with an absolute pressure of 0.23 atm for 3 min before release. The infiltrated leaves were allowed to air dry under a fume hood for 1 h and the plants were then maintained at 24° C. and 60% humidity for 7 days with a 16-h photoperiod (10,000 lux). The leaves were harvested and stored at −80° C.

VLPs were recovered from ˜100 g of agroinfiltrated tissue by pulverization and homogenization in three volumes (300 ml) of cold 0.1 M sodium phosphate buffer (pH 7.0) containing 2% polyvinylpolypyrrolidone (Sigma-Aldrich). The cell lysate was filtered through Miracloth to remove debris and centrifuged (12,000×g, 20 min, 4° C.). The supernatant was supplemented with 0.2 M NaCl and 8% (w/v) PEG (MW 8000) and stirred overnight at 4° C. The solution was centrifuged (14,000×g, 15 min, 4° C.) and the pellet was redissolved in 50 ml 10 mM sodium phosphate buffer (pH 7.0) overnight. The next day, the solution was centrifuged (12,000×g, 15 min, 4° C.) and the cleared supernatant was pelleted by ultracentrifugation (169,000×g, 3 h, 4° C.). The pellet was resuspended in 2 ml 0.1 M sodium phosphate buffer (pH 7.0) overnight at 4° C. Finally, the VLPs were purified by ultracentrifugation on a 30% sucrose cushion (133,000×g, 3 h, 4° C.). The VLP band was collected, pelleted by ultracentrifugation (169,000×g, 3 h, 4° C.), and the pellet was resuspended in 0.1 M sodium phosphate buffer (pH 7.0). The total protein concentration was determined using a BCA assay (Thermo Fisher Scientific).

### Electrophoretic Characterization of VLPs

The VLPs were characterized by denaturing sodium dodecyl sulfate polyacrylamide gel electrophoresis (SDS-PAGE) and agarose gel electrophoresis. For SDS-PAGE, Applicant loaded 10-μg and 20-μg samples of total protein per lane on NuPAGE 4-12% gels and fractionated them for 37 min at 200 mV. The gels were stained with GelCode Blue Safe Protein Stain (Thermo Fisher Scientific). For agarose gel electrophoresis, 20-μg samples of total protein were loaded onto 1.2% agarose gels and fractionated for 1 h at 80 mV. For nucleic acid identification the gel was stained with GelRed (Gold Biotechnology) and for protein identification the gel was stained with Coomassie Brilliant Blue. All gels were imaged using the ProteinSimple FluorChem R imaging system.

### Transmission Electron Microscopy

Carbon-coated copper negative stain grids were glow-discharged for 30 s (easiGlow) before adding 10-μl samples of total protein, incubating for 2 min and blotting away excess liquid. The grids were washed twice in Milli-Q water before applying two lots of 10 μl 2% (w/v) uranyl acetate, leaving the stain for 30 s each time before blotting. The grids were air dried and analyzed on a JEOL JEM-1400 series 120 kV Transmission Electron Microscope. The size of the CiLV-C VLPs was determined analyzing three images at different magnifications (60,000×, 80,000× and 100,000×) and measuring the diameter of 100 particles from five random fields (20 particles/field) in each image. The diameter of the 300 particles in total was presented as a frequency-size distribution histogram and the polydispersity index (PDI) was calculated as follows:

PDI=(standard deviation/mean diameter size)²

### Size Exclusion Chromatography

CiLV-C VLPs were analyzed by size exclusion chromatography (SEC) using an AKTA Pure system fitted with a Superose 6 Increase 10/300 GL column (GE Healthcare). Samples (500 μg total protein) were analyzed at a flow rate of 0.5 ml/min using 0.1 M sodium phosphate buffer (pH 7.0).

### Cloning and Expression of the CiLV-C p29 Gene

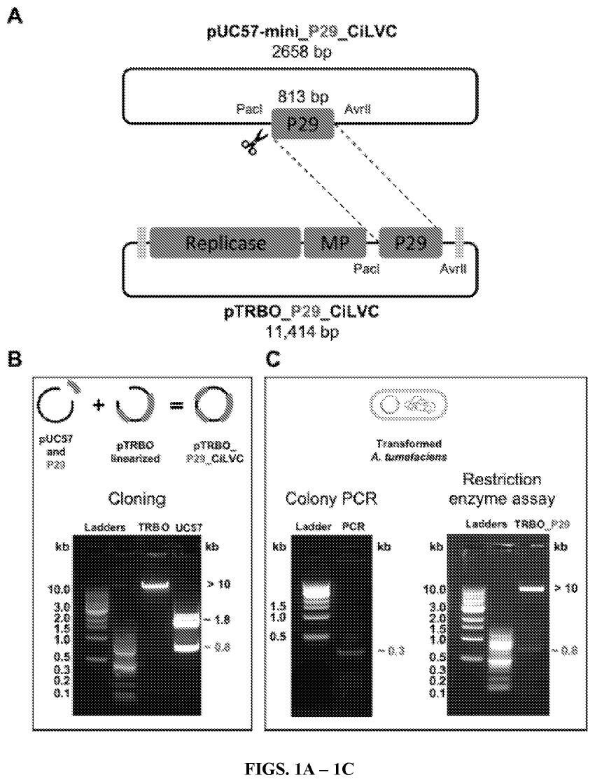

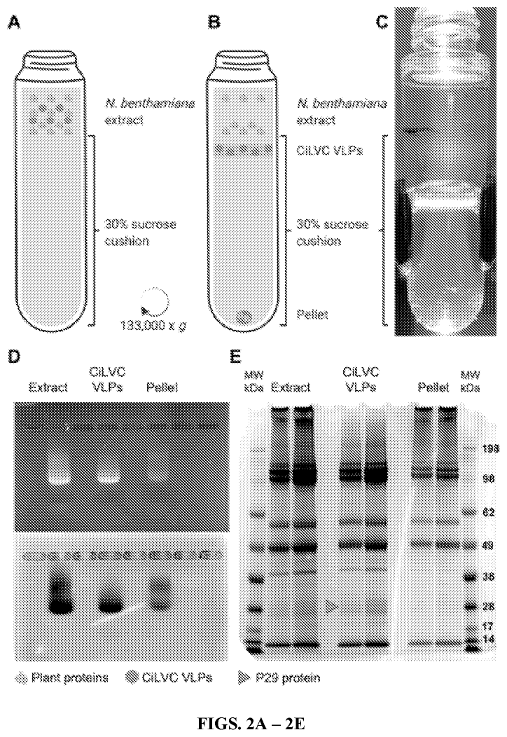

The codon-optimized CiLV-C p29 sequence (SEQ ID NO: 2, FIG. 6) was synthesized by GenScript Biotech and provided in vector pUC57-mini_P29_CiLVC. Applicant transferred the p29 ORF to the pTRBO vector, which contains the TMV replicase (SEQ ID NO: 5) and movement protein genes (SEQ ID NO: 6) and is widely used for the overexpression of recombinant proteins in plants (FIG. 1A). The recombinant vector pTRBO_P29_CiL VC was introduced into A. tumefaciens strain GV3101 by electroporation, and the presence of the insert was confirmed by colony PCR (FIG. 1B) and a diagnostic restriction assay, which yielded a product of ˜813 bp in addition to the >10-kb linearized pTRBO backbone (FIG. 1C).

The leaves of 6-week-old N. benthamiana plants were vacuum infiltrated with the suspension of A. tumefaciens and left to express the p29 protein for 7 days. VLPs were then recovered from leaf extracts by ultracentrifugation over a 30% sucrose cushion (FIGS. 2A-2C). Agarose gel electrophoresis showed that extracts before and after ultracentrifugation contained comigrating nucleic acid and protein bands (FIG. 2D). VLPs lack genomic RNA, but often package random host RNA molecules based on nonspecific electrostatic interactions with the nucleic acid backbone [23], given the absence of a common packaging motif. SDS-PAGE under denaturing conditions confirmed the presence of the 29-kDa capsid protein in the crude extract before ultracentrifugation, and in the VLP band, but not in the pellet (FIG. 2E).

### Size and Morphology of the CiLV-C VLPs

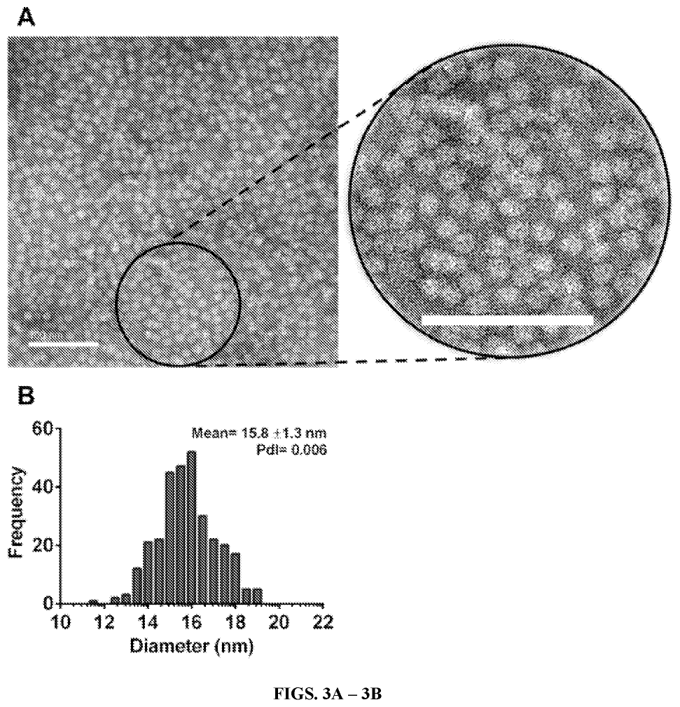

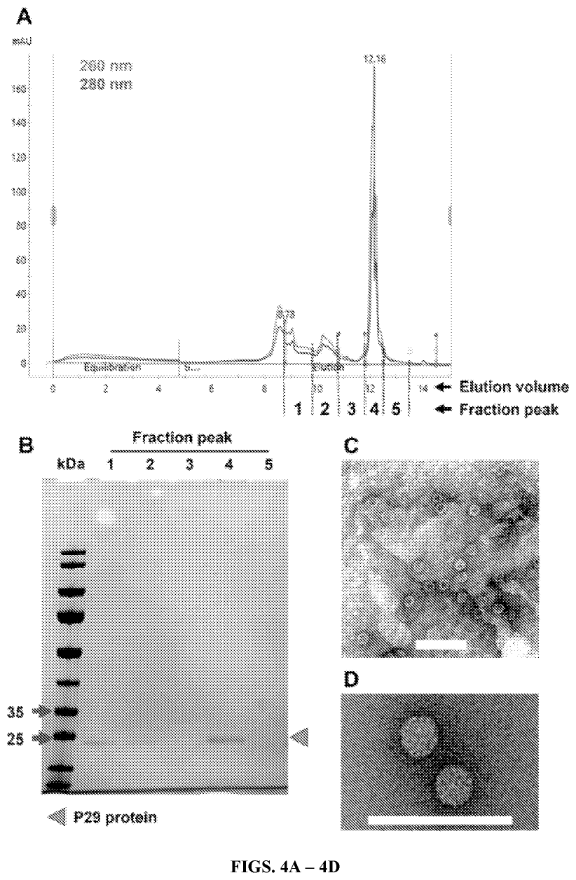

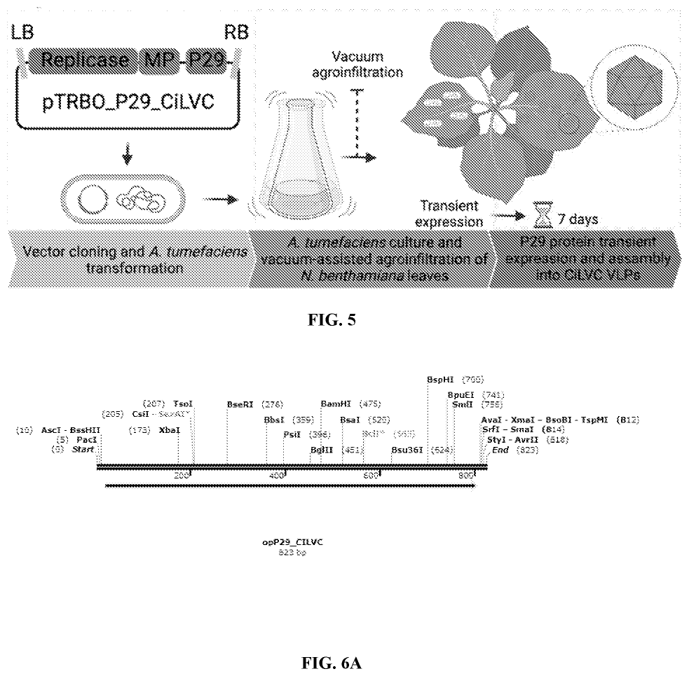

The original morphological description of CiLV-C referred to short bacilliform particles (60-70 nm×110-120 nm) in the cisternae of the ER, as well as electron-dense viroplasms distributed in the cytoplasm [24]. However, the analysis of TEM images revealed an icosahedral morphology with T=1 symmetry (FIG. 3A) and the VLPs were also smaller in size than the native particles (15.8±1.3 nm) with a PDI of 0.006 (FIG. 3B). SEC analysis revealed one major peak and two minor peaks among five fractions (FIG. 4A). The analysis of all five fractions by SDS-PAGE under denaturing conditions revealed the presence of the anticipated˜29-kDa capsid protein band in the fractions representing all three peaks (FIG. 4B). Applicant was unable to recover sufficient materials to perform further analysis of peaks 1 and 2, which are likely to represent aggregated VLPs or larger assemblies of p29. Applicant analyzed the major fraction (peak 4) by TEM and confirmed the presence of icosahedral particles (FIG. 4C, FIG. 4D).

The goal was to produce bacilliform VLPs larger than the typical 30-nm icosahedrons formed by other plant viruses. The CiLV-C VLPs did not assemble into the anticipated bacilliform particles; the icosahedral particles with T=1 symmetry were smaller than usual (˜15 nm) but offer an opportunity to test for size-specific behavior during cell uptake and in vivo trafficking. VLPs that self-assemble from capsid proteins expressed in microbes, animal cells, plant cells or cell-free systems often resemble the structure of the parent virus, but this is not always the case. For example, in contrast to most members of the Alfamovirus genus, many strains of alfalfa mosaic virus (AIMV) are composed of bacilliform particles, but expressing the coat protein in Escherichia coli produces icosahedral particles with T=1 symmetry similar to those Applicant observed for CiLV-C [25, 26]. This is likely to reflect the absence of viral genomic RNA, which normally coordinates the assembly of coat protein subunits. Indeed. AlMV can form particles with various morphologies, including bacilliform, icosahedral and long tubular structures resembling the cross-section of the icosahedral capsid (with or without icosahedral end caps) depending on the presence/absence of nucleic acids [27] and the specific type: heterologous virus genome, calf thymus DNA, yeast total RNA, or poly (A) RNA [28, 29]. These morphologies can also be replicated by limited trypsin digestion [30] and by coat protein mutants that influence the formation of coat protein dimers (as the basic unit of capsid assembly) and the choice between the formation of pentamers or hexamers [26, 31]. It is possible that peaks 1 and 2 in Applicant's SEC eluate contained higher-order assemblies, and that these could be scaled up and isolated by adjusting the expression conditions, including the co-expression of other structural proteins.

### Experimental Conclusions

Applicant expressed the CiLV-C p29 capsid protein in N. benthamiana and confirmed that it self-assembles into stable VLPs, albeit differing in size and morphology from the native CiLV-C particles. Such differences are often observed when some of the components required to assemble native particles are missing (in this case, the most likely candidates are the native RNA1 and RNA2, but potentially also one or more of the five additional CiLV-C proteins). The production of CiLV-C VLPs provides insight into the assembly mechanism and can facilitate the development of more effective countermeasures against citrus leprosis based on the direct inhibition of the viral replication cycle. CiLV-C VLPs could also be developed as delivery platforms for drugs, vaccines, and imaging reagents.

### Sequence Listing—Table 3

Syn* intends Synthetic construct

Mol Type intends molecule type

| | Sequence | | Mol | Or- |
|---|---|---|---|---|
| Seq ID | Name | Sequences | Type | ganism |
| SEQ ID | p29 gene | TTAATTAAGGCGCGCCATGTCTATTGTTACTTTTA | DNA | Syn* |
| NO: 1 | | CTCTTACTGATCCTTCTTCTGCTCTTATTGCTGAA | | |
| | | ATTATGCAAGCTATTGAAAGACATAACGTTTCTG | | |
| | | TTCTTGAAGGTCTTAGAGATATTTCTAAGCCTACT | | |
| | | AAGAAGAAGCAACAATCTCAACCTCAACAACTTT | | |
| | | CTAGAGCTTCTGCTAGACCTCAACAACTTCAACC | | |
| | | TGGTCCTTCTGGTTATCAAGCTAAGAAGCCTGCT | | |
| | | AAGCAAAAGGCTGAAGTTGTTAAGCCTAAGCAA | | |
| | | AAGCAACTTGCTCCTCCTATTAATAAGAAGGCTG | | |
| | | CTAAGGCTAAGCTTTATGGTCTTGAACAACATTG | | |
| | | TCCTAAGTATGCTGAAGCTAAGGGTCTTCAAAAG | | |
| | | CAAATTGGTATGACTTATTATAAGATTTCTGAACC | | |
| | | TTACGCTCTTCCTGATTTTAAGGTTATGGAAGCTT | | |
| | | CTGAAGATCTTGTTGCTGTTTCTGAAAAGGATCCT | | |
| | | ATGGGTTCTTTTGAAAAGAGACTTTATTCTATGGG | | |
| | | TTTTCCTAAGAGACCTATTAAGAACGTTGTTCCTG | | |
| | | TTTTTGAATTTTCTGATCATTATATTGTTGTTTTTT | | |
| | | TTCCTGGTTCTAATGCTGAAATTGTTAAGAATGTT | | |
| | | CCTAAGGATTCTGTTTCTGATTATGCTGAAGCTCA | | |
| | | ACTTGCTGCTCTTCTTGCTGCTAGACAACAAATTA | | |
| | | ATCAAATTCATGAACTTGGTGATATTCTTCCTACT | | |
| | | AATTATCTTAATGTTCTTGATTCTGGTACTCAAGA | | |
| | | TGTTGTTGTTTCTGATGAAGAAGATGATTCTGATT | | |
| | | CTGCTCAATAATAGCCCGGGCCTAGG | | |
| SEQ ID | p29 | MSIVTFTLTDPSSALIAEIMQAIERHNVSVPEGLRDIS | AA | Syn* |
| NO: 2 | poly- | KPTKKKQQSQPQQLSRASARPQQLQPGPSGYQAKK | | |
| | peptide | PAKQKAEVVKPKQKQLAPPINKKAAKAKLYGLEQ | | |
| | | HCPKYAEAKGLQKQIGMTYYKISEPYALPDFKVME | | |
| | | ASEDLVAVSEKD | | |
| | | PMGSFEKRLYSMGFPKRPIKNVVPVFEFSDHYIVVF | | |
| | | FPGSNAEIVKNVPKDSVSDYAEAQLAALLAARQQI | | |
| | | NQIHELGDILPTNYLNVLDSGTQDVVVSDEEDDSDS | | |
| | | AQ | | |
| SEQ ID | TMV Ω | acaattaccaacaacaacaaacaacagacaacattacaattactatttacaattac | DNA | Syn* |
| NO: 3 | | | | |
| SEQ ID | Replicase | “atggcatacacacagacagctaccacatcagctttgctggacactgtccgagga | DNA | Syn* |
| NO: 4 | | aacaactccttggtcaatgatctagcaaagcgtcgtctttacgacacagcggttga | | |
| | | agagtttaacgctcgtgaccgcaggcccaaggtgaacttttcaaaagtaataagc | | |
| | | gaggagcagacgcttattgctacccgggcgtatccagaattccaaattacattttat | | |
| | | aacacgcaaaatgccgtgcattcgcttgcaggtggattgcgatctttagaactgga | | |
| | | atatctgatgatgcaaattccctacggatcattgacttatgacataggcgggaatttt | | |
| | | gcatcgcatctgttcaagggacgagcatatgtacactgctgcatgcccaacctgg | | |
| | | acgttcgagacatcatgcggcacgaaggccagaaagacagtattgaactatacct | | |
| | | ttctaggctagagagaggggggaaaacagtccccaacttccaaaaggaagcattt | | |
| | | gacagatacgcagaaattcctgaagacgctgtctgtcacaatactttccagacatg | | |
| | | cgaacatcagccgatgcagcaatcaggcagagtgtatgccattgcgctacacag | | |
| | | catatatgacataccagccgatgagttcggggcggcactcttgaggaaaaatgtc | | |
| | | catacgtgctatgccgctttccacttctccgagaacctgcttcttgaagattcatgcg | | |
| | | tcaatttggacgaaatcaacgcgtgtttttcgcgcgatggagacaagttgaccttttc | | |
| | | ttttgcatcagagagtactcttaattactgtcatagttattctaatattcttaagtat | | |
| | | gtgtgcaaaacttacttcccggcctctaatagagaggtttacatgaaggagtttttag | | |
| | | tcaccagagttaatacctggttttgtaagttttctagaatagatacttttcttttgta | | |
| | | caaaggtgtggcccataaaagtgtagatagtgagcagttttatactgcaatggaagac | | |
| | | gcatggcattacaaaaagactcttgcaatgtgcaacagcgagagaatcctccttgagg | | |
| | | attcatcatcagtcaattactggtttcccaaaatgagggatatggtcatcgtaccatt | | |
| | | attcgacatttctttggagactagtaagaggacgcgcaaggaagtcttagtgtccaag | | |
| | | gatttcgtgtttacagtgcttaaccacattcgaacataccaggcgaaagctcttacat | | |
| | | acgcaaatgttttgtccttcgtcgaatcgattcgatcgagggtaatcattaacggtgt | | |
| | | gacagcgaggtccgaatgggatgtggacaaatctttgttacaatccttgtccatgac | | |
| | | gttttacctgcatactaagcttgccgttctaaaggatgacttactgattagcaagttt | | |
| | | agtctcggttcgaaaacggtgtgccagcatgtgtgggatgagatttcgctggcgttt | | |
| | | gggaacgcatttccctccgtgaaagagaggctcttgaacaggaaacttatcagag | | |
| | | tggcaggcgacgcattagagatcagggtgcctgatctatatgtgaccttcc | | |
| | | acgacagattagtgactgagtacaaggcctctgtggacatgcctgcgcttgacatt | | |
| | | aggaagaagatggaagaaacggaagtgatgtacaatgcactttcagaattatcgg | | |
| | | tgttaagggagtctgacaaattcgatgttgatgttttttcccagatgtgccaatcttt | | |
| | | ggaagttgacccaatgacggcagcgaaggttatagtcgcggtcatgagcaatgag | | |
| | | agcggtctgactctcacatttgaacgacctactgaggcgaatgttgcgctagcttta | | |
| | | caggatcaagagaaggcttcagaaggtgcattggtagttacctcaagagaagttg | | |
| | | aagaaccgtccatgaagggttcgatggccagaggagagttacaattagctggtct | | |
| | | tgctggagatcatccggaatcgtcctattctaagaacgaggagatagagtctttag | | |
| | | agcagtttcatatggcgacggcagattcgttaattcgtaagcagatgagctcgattg | | |
| | | tgtacacgggtccgattaaagttcagcaaatgaaaaactttatcgatagcctggtag | | |
| | | catcactatctgctgcggtgtcgaatctcgtcaagatcctcaaagatacagctgcta | | |
| | | ttgaccttgaaacccgtcaaaagtttggagtcttggatgttgcatctaggaagtggtt | | |
| | | aatcaaaccaacggccaagagtcatgcatggggtgttgttgaaacccacgcgag | | |
| | | gaagtatcatgtggcgcttttggaatatgatgagcagggtgtggtgacatgcgatg | | |
| | | attggagaagagtagctgttagctctgagtctgttgtttattccgacatggcgaaact | | |
| | | cagaactctgcgcagactgcttcgaaacggagaaccgcatgtcagtagcgcaaa | | |
| | | ggttgttcttgtggacggagttccgggctgtggaaaaaccaaagaaattctttcca | | |
| | | gggttaattttgatgaagatctaattttagtacctgggaagcaagccgcggaaatga | | |
| | | tcagaagacgtgcgaattcctcagggattattgtggccacgaaggacaacgttaa | | |
| | | aaccgttgattctttcatgatgaattttgggaaaagcacacgctgtcagttcaagag | | |
| | | gttattcattgatgaagggttgatgttgcatactggttgtgttaattttcttgtggcg | | |
| | | atgtcattgtgcgaaattgcatatgtttacggagacacacagcagattccatacatca | | |
| | | atagagtttcaggattcccgtaccccgcccattttgccaaattggaagttgacgaggt | | |
| | | ggagacacgcagaactactctccgttgtccagccgatgtcacacattatctgaaca | | |
| | | ggagatatgagggctttgtcatgagcacttcttcggttaaaaagtctgtttcgcagg | | |
| | | agatggtcggcggagccgccgtgatcaatccgatctcaaaacccttgcatggcaa | | |
| | | gatcttgacttttacccaatcggataaagaagctctgctttcaagagggtattcagat | | |
| | | gttcacactgtgcatgaagtgcaaggcgagacatactctgatgtttcactagttagg | | |
| | | ttaacccctacaccggtctccatcattgcaggagacagcccacatgttttggtcgca | | |
| | | ttgtcaaggcacacctgttcgctcaagtactacactgttgttatggatcctttagtta | | |
| | | gtatcattagagatctagagaaacttagctcgtacttgttagatatgtataaggtcga | | |
| | | tgcaggaacacaatagcaattacagattgactcggtgttcaaaggttccaatcttttt | | |
| | | gttgcagcgccaaagactggtgatatttctgatatgcagttttactatgataagtgtc | | |
| | | tcccaggcaacagcaccatgatgaataattttgatgctgttaccatgaggttgactga | | |
| | | catttcattgaatgtcaaagattgcatattggatatgtctaagtctgttgctgcgcct | | |
| | | aaggatcaaatcaaaccactaatacctatggtacgaacggcggcagaaatgccacg | | |
| | | ccagactggactattggaaaatttagtggcgatgattaaaagaaactttaacgcac | | |
| | | ccgagttgtctggcatcattgatattgaaaatactgcatctttggttgtagataagtt | | |
| | | ttttgatagttatttgcttaaagaaaaaagaaaaccaaataaaaatgtttctttgttc | | |
| | | agtagagagtctctcaatagatggttagaaaagcaggaacaggtaacaataggccagc | | |
| | | tcgcagattttgattttgtggatttgccagcagttgatcagtacagacacatgattaa | | |
| | | agcacaacccaaacaaaagttggacacttcaatccaaacggagtacccggctttgc | | |
| | | agacgattgtgtaccattcaaaaaagatcaatgcaatattcggcccgttgtttagtga | | |
| | | gcttactaggcaattactggacagtgttgattcgagcagatttttgtttttcacaaga | | |
| | | aagacaccagcgcagattgaggatttcttcggagatctcgacagtcatgtgccgat | | |
| | | ggatgtcttggagctggatatatcaaaatacgacaaatctcagaatgaattccactg | | |
| | | tgcagtagaatacgagatctggcgaagattgggtttcgaagacttcttgggagaa | | |
| | | gtttggaaacaagggcatagaaagaccaccctcaaggattataccgcaggtataa | | |
| | | aaacttgcatctggtatcaaagaaagagcggggacgtcacgacgttcattggaaa | | |
| | | cactgtgatcattgctgcatgtttggcctcgatgcttccgatggagaaaataatcaa | | |
| | | aggagccttttgcggtgacgatagtctgctgtactttccaaagggttgtgagtttcc | | |
| | | ggatgtgcaacactccgcgaatcttatgtggaattttgaagcaaaactgtttaaaaa | | |
| | | acagtatggatacttttgcggaagatatgtaatacatcacgacagaggatgcattgt | | |
| | | gtattacgatcccctaaagttgatctcgaaacttggtgctaaacacatcaaggattg | | |
| | | ggaacacttggaggagttcagaaggtctctttgtgatgttgctgtttcgttgaacaat | | |
| | | tgtgcgtattacacacagttggacgacgctgtatgggaggttcataagaccgccc | | |
| | | ctccaggttcgtttgtttataaaagtctggtgaagtatttgtctgataaagttctttt | | |
| | | tagaagtttgtttatagatggctctagttgttaa” | | |
| SEQ ID | TMV MP | atggctctagttgttaaaggaaaagtgaatatcaatgagtttatcgacctgacaaaa | DNA | Syn* |
| NO: 5 | poly- | atggagaagatcttaccgtcgatgtttacccctgtaaagagtgttatgtgttccaaag | | |
| | nucleotide | ttgataaaataatggttcatgagaatgagtcattgtcaggggtgaaccttcttaaag | | |
| | | gagttaagcttattgatagtggatacgtctgtttagccggtttggtcgtcacgggcg | | |
| | | agtggaacttgcctgacaattgcagaggaggtgtgagcgtgtgtctggtggacaa | | |
| | | aaggatggaaagagccgacgaggccactctcggatcttactacacagcagctgc | | |
| | | aaagaaaagatttcagttcaaggtcgttcccaattatgctataaccacccaggacg | | |
| | | cgatgaaaaacgtctggcaagttttagttaatattagaaatgtgaagatgtcagcgg | | |
| | | gtttctgtccgctttctctggagtttgtgtcggtgtgtattgtttatagaaataatat | | |
| | | aaaattaggtttgagagagaagattacaaacgtgagagacggagggcccatggaact | | |
| | | tacagaagaagtcgttgatgagttcatggaagatgtccctatgtcgatcaggcttgc | | |
| | | aaagtttcgatctcgaaccggaaaaaagagtgatgtccgcaaagggaaaaatagt | | |
| | | agtagtgatcggtcagtgccgaacaagaactatagaaatgttaaggattttggagg | | |
| | | aatgagttttaaaaagaataatttaatcgatgatgattcggaggctactgtcgccga | | |
| | | atcggattcgttttaa | | |
| SEQ ID | TMV | “MALVVKGKVNINEFIDLTKMEKILPSMFTPVKSVM | AA | Syn* |
| NO: 6 | movement | CSKVDKIMVHENESLSGVNLLKGVKLIDSGYVCLA | | |
| | protein | GLVVTGE | | |
| | (MP, | WNLPDNCRGGVSVCLVDKRMERADEATLGSYYTA | | |
| | poly- | AAKKRFQFKVVPNYAITTQDAMKNVWQVLVNIRN | | |
| | peptide) | VKMSAGFCP | | |
| | | LSLEFVSVCIVYRNNIKLGLREKITNVRDGGPMELT | | |
| | | EEVVDEFMEDVPMSIRLAKFRSRTGKKSDVRKGKN | | |
| | | SSSDRS | | |
| | | VPNKNYRNVKDFGGMSFKKNNLIDDDSEATVAESD | | |
| | | SF” | | |
| SEQ ID | CiLV-C- | “ATGTCTATTGTTACTTTTACTCTTACTGATCCTTC | DNA | Syn* |
| NO: 7 | poly- | TTCTGCTCTTATTGCTGAAATTATGCAAGCTATTG | | |
| | nucleotide | AAAGACATAACGTTTCTGTTCCTGAAGGTCTTAG | | |
| | | AGATATTTCTAAGCCTACTAAGAAGAAGCAACAA | | |
| | | TCTCAACCTCAACAACTTTCTAGAGCTTCTGCTAG | | |
| | | ACCTCAACAACTTCAACCTGGTCCTTCTGGTTATC | | |
| | | AAGCTAAGAAGCCTGCTAAGCAAAAGGCTGAAG | | |
| | | TTGTTAAGCCTAAGCAAAAGCAACTTGCTCCTCC | | |
| | | TATTAATAAGAAGGCTGCTAAGGCTAAGCTTTAT | | |
| | | GGTCTTGAACAACATTGTCCTAAGTATGCTGAAG | | |
| | | CTAAGGGTCTTCAAAAGCAAATTGGTATGACTTA | | |
| | | TTATAAGATTTCTGAACCTTACGCTCTTCCTGATT | | |
| | | TTAAGGTTATGGAAGCTTCTGAAGATCTTGTTGCT | | |
| | | GTTTCTGAAAAGGATCCTATGGGTTCTTTTGAAA | | |
| | | AGAGACTTTATTCTATGGGTTTTCCTAAGAGACCT | | |
| | | ATTAAGAACGTTGTTCCTGTTTTTGAATTTTCTGA | | |
| | | TCATTATATTGTTGTTTTTTTTCCTGGTTCTAATGC | | |
| | | TGAAATTGTTAAGAATGTTCCTAAGGATTCTGTTT | | |
| | | CTGATTATGCTGAAGCTCAACTTGCTGCTCTTCTT | | |
| | | GCTGCTAGACAACAAATTAATCAAATTCATGAAC | | |
| | | TTGGTGATATTCTTCCTACTAATTATCTTAATGTT | | |
| | | CTTGATTCTGGTACTCAAGATGTTGTTGTTTCTGA | | |
| | | TGAAGAAGATGATTCTGATTCTGCTCAATAA” | | |
| SEQ ID | CiLV-C- | “MSIVTFTLTDPSSALIAEIMQAIERHNVSVPEGLRDI | AA | Syn* |
| NO: 8 | poly- | SKPTKKKQQSQPQQLSRASARPQQLQPGPSGYQAK | | |
| | peptide | KPAKQ | | |
| | | KAEVVKPKQKQLAPPINKKAAKAKLYGLEQHCPK | | |
| | | YAEAKGLQKQIGMTYYKISEPYALPDFKVMEASED | | |
| | | LVAVSEKD | | |
| | | PMGSFEKRLYSMGFPKRPIKNVVPVFEFSDHYIVVF | | |
| | | FPGSNAEIVKNVPKDSVSDYAEAQLAALLAARQQI | | |
| | | NQIHEL | | |
| | | GDILPTNYLNVLDSGTQDVVVSDEEDDSDSAQ” | | |
| SEQ ID | Comp- | CCTAGGCCCGGGCTATTATTGAGCAGAATCAGAA | DNA | Syn* |
| NO: 9 | limentary | TCATCTTCTTCATCAGAAACAACAACATCTTGAGT | | |
| | strand of | ACCAGAATCAAGAACATTAAGATAATTAGTAGGA | | |
| | p29 gene | AGAATATCACCAAGTTCATGAATTTGATTAATTT | | |
| | (5′-3′) | GTTGTCTAGCAGCAAGAAGAGCAGCAAGTTGAGC | | |
| | | TTCAGCATAATCAGAAACAGAATCCTTAGGAACA | | |
| | | TTCTTAACAATTTCAGCATTAGAACCAGGAAAAA | | |
| | | AAACAACAATATAATGATCAGAAAATTCAAAAA | | |
| | | CAGGAACAACGTTATTAATAGGTCTCTTAGGAAA | | |
| | | ACCCATAGAATAAAGTCTCTTTTCAAAAGAACCC | | |
| | | ATAGGATCCTTTTCAGAAACAGCAACAAGATCTT | | |
| | | CAGAAGCTTCCATAACCTTAAAATCAGGAAGAGC | | |
| | | GTAAGGTTCAGAAATCTTATAATAAGTCATACCA | | |
| | | ATTTGCTTTTGAAGACCCTTAGCTTCAGCATACTT | | |
| | | AGGACAATGTTGTTCAAGACCATAAAGCTTAGCC | | |
| | | TTAGCAGCCTTCTTATTAATAGGAGGAGCAAGTT | | |
| | | GCTTTTGCTTAGGCTTAACAACTTCAGCCTTTTGC | | |
| | | TTAGCAGGCTTCTTAGCTTGATAACCAGAAGGAC | | |
| | | CAGGTTGAAGTTGTTGAGGTCTAGCAGAAGCTCT | | |
| | | AGAAAGTTGTTGAGGTTGAGATTGTTGCTTCTTCT | | |
| | | TAGTAGGCTTAGAAATATCTCTAAGACCTTCAGG | | |
| | | AACAGAAACGTTATGTCTTTCAATAGCTTGCATA | | |
| | | ATTTCAGCAATAAGAGCAGAAGAAGGATCAGTA | | |
| | | AGAGTAAAAGTAACAATAGACATGGCGCGCCTTA | | |
| | | ATTAA | | |
| SEQ ID | Comp- | gtaattgtaaatagtaattgtaatgttgtctgttgtttgttgttgttggtaattgt | DNA | Syn* |
| NO: 10 | limentary | | | |
| | strand of | | | |
| | TMV Ω | | | |
| | (5′-3′) | | | |
| SEQ ID | Comp- | aattgttgatctcggtagatatttgtttgaagatttttcttgaaatagtctgtttatg | DNA | Syn* |
| NO: 11 | limentary | aagtggtctgaaaatatttgtttgcttggacctccccgccagaatacttggagggtat | | |
| | strand of | gtcgcagcaggttgacacacattatgcgtgttaacaagttgctttgtcgttgtagtgt | | |
| | Replicase | ttctctggaagacttgaggaggttcacaagggttaggaactacacaaatcgtggttca | | |
| | (5′-3′) | aagctctagttgaaatcccctagcattatgtgttacgtaggagacagcactacataat | | |
| | | gtatagaaggcgttttcataggtatgacaaaaaatttgtcaaaacgaagttttaaggt | | |
| | | gtattctaagcgcctcacaacgtgtaggcctttgagtgttgggaaacctttcatgtc | | |
| | | gtctgatagcagtggcgttttccgaggaaactaataaaagaggtagccttcgtagc | | |
| | | tccggtttgtacgtcgttactagtgtcacaaaggttacttgcagcactgcaggggc | | |
| | | gagaaagaaactatggtctacgttcaaaaatatggacgccatattaggaactccca | | |
| | | ccagaaagatacgggaacaaaggtttgaagagggttcttcagaagctttgggtta | | |
| | | gaagcggtctagagcataagatgacgtgtcaccttaagtaagactctaaacagcat | | |
| | | aaaactatataggtcgaggttctgtaggtagccgtgtactgacagctctagaggctt | | |
| | | ctttaggagttagacgcgaccacagaaagaacactttttgtttttagacgagcttagt | | |
| | | tgtgacaggtcattaacggatcattcgagtgatttgttgcccggcttataacgtaact | | |
| | | agaaaaaacttaccatgtgttagcagacgtttcggcccatgaggcaaacctaactt | | |
| | | cacaggttgaaaacaaacccaacacgaaattagtacacagacatgactagttgac | | |
| | | gaccgtttaggtgttttagttttagacgctcgaccggataacaatggacaaggacg | | |
| | | aaaagattggtagataactctctgagagatgacttgtttctttgtaaaaataaaccaa | | |
| | | aagaaaaaagaaattcgtttattgatagttttttgaatagatgttggtttctacgtca | | |
| | | taaaagttatagttactacggtctgttgagcccacgcaatttcaaagaaaattagtag | | |
| | | cggtgatttaaaaggttatcaggtcagaccgcaccgtaaagacggcggcaagcat | | |
| | | ggtatccataatcaccaaactaaactaggaatccgcgtcgttgtctgaatctgtata | | |
| | | ggttatacgttagaaactgtaagttactttacagtcagttggagtaccattgtcgtag | | |
| | | ttttaataagtagtaccacgacaacggaccctctgtgaatagtatcattttgacgtat | | |
| | | agtctttatagtggtcagaaaccgcgacgttgtttttctaaccttggaaacttgtggc | | |
| | | tcagttagacattaacgataacacaaggacgtagctggaatatgtatagattgttcat | | |
| | | gctcgattcaaagagatctagagattactatgattgatttcctaggtattgttgtcac | | |
| | | atcatgaactcgcttgtccacacggaactgttacgctggttttgtacacccgacagag | | |
| | | gacgttactacctctggccacatccccaattggattgatcactttgtagtctcataca | | |
| | | gagcggaacgtgaagtacgtgtcacacttgtagacttatgggagaactttcgtctc | | |
| | | gaagaaataggctaacccattttcagttctagaacggtacgttcccaaaactctagc | | |
| | | ctaactagtgccgccgaggcggctggtagaggacgctttgtctgaaaaattggctt | | |
| | | cttcacgagtactgtttcgggagtatagaggacaagtctattacacactgtagccga | | |
| | | cctgttgcctctcatcaagacgcacagaggtggagcagttgaaggttaaaccgttt | | |
| | | tacccgccccatgcccttaggactttgagataactacataccttagacgacacaca | | |
| | | gaggcatttgtatacgttaaagcgtgttactgtagcggtgttcttttaattgtgttgg | | |
| | | tcatacgttgtagttgggaagtagttacttattggagaacttgactgtcgcacacgaa | | |
| | | aagggttttaagtagtactttcttagttgccaaaattgcaacaggaagcaccggtgtt | | |
| | | attagggactccttaagcgtgcagaagactagtaaaggcgccgaacgaagggtcc | | |
| | | atgattttaatctagaagtagttttaattgggacctttcttaaagaaaccaaaaaggt | | |
| | | gtcgggccttgaggcaggtgttcttgttggaaacgcgatgactgtacgccaagagg | | |
| | | caaagcttcgtcagacgcgtctcaagactcaaagcggtacagccttatttgttgtct | | |
| | | gagtctcgattgtcgatgagaagaggttagtagcgtacagtggtgtgggacgagt | | |
| | | agtataaggttttcgcggtgtactatgaaggagcgcacccaaagttgttgtggggt | | |
| | | acgtactgagaaccggcaaccaaactaattggtgaaggatctacgttgtaggttct | | |
| | | gaggtttgaaaactgcccaaagttccagttatcgtcgacatagaaactcctagaact | | |
| | | gctctaagctgtggcgtcgtctatcactacgatggtccgatagctatttcaaaaagt | | |
| | | aaacgacttgaaattagcctgggcacatgtgttagctcgagtagacgaatgcttaat | | |
| | | tgcttagacggcagcggtatactttgacgagatttctgagatagaggagcaagaat | | |
| | | cttatcctgctaaggcctactagaggtcgttctggtcgattaacattgagaggagac | | |
| | | cggtagcttgggaagtacctgccaagaagttgaagagaactccattgatggttac | | |
| | | gtggaagacttcggaagagaactaggacatttcgatcgcgttgtaagcggagtca | | |
| | | tccagcaagtttacactctcagtctggcgagagtaacgagtactggcgctgatatt | | |
| | | ggaagcgacggcagtaacccagttgaaggtttctaaccgtgtagacccttttttgta | | |
| | | gttgtagcttaaacagtctgagggaattgtggctattaagactttcacgtaacatgta | | |
| | | gtgaaggcaaagaaggtagaagaaggattacagttcgcgtccgtacaggtgtct | | |
| | | ccggaacatgagtcagtgattagacagcaccttccagtgtatatctagtccgtggg | | |
| | | actagagattacgcagcggacggtgagactattcaaaggacaagttctcggaga | | |
| | | gaaagtgcctccctttacgcaagggtttgcggtcgctttagagtagggtgtgtacg | | |
| | | accgtgtggcaaaagcttggctctgatttgaacgattagtcattcagtaggaaatctt | | |
| | | gccgttcgaatcatacgtccattttgcagtacctgttcctaacattgtttctaaacag | | |
| | | gtgtagggtaagcctggagcgacagtgtggcaattactaatgggagctagcttagc | | |
| | | taagctgcttcctgttttgtaaacgcatacattctcgaaagcggaccatacaagctta | | |
| | | caccaattcgtgacatttgtgctttaggaacctgtgattctgaaggaacgcgcagg | | |
| | | agaatgatcagaggtttctttacagcttattaccatgctactggtatagggagtaaaa | | |
| | | ccctttggtcattaactgactactacttaggagttcctcctaagagagcgacaacgt | | |
| | | gtaacgttctcagaaaaacattacggtacgcagaaggtaacgtcatattttgacga | | |
| | | gtgatagatgtgaaaatacccggtgtggaaacatgttttcttttcatagataagatct | | |
| | | tttgaatgttttggtccataattgagaccactgatttttgaggaagtacatttggaga | | |
| | | gataatctccggcccttcattcaaaacgtgtgtatgaattcttataatcttattgata | | |
| | | ctgtcattaattctcatgagagactacgttttcttttccagttgaacagaggtagcgc | | |
| | | gctttttgtgcgcaactaaagcaggtttaactgcgtacttagaagttcttcgtccaag | | |
| | | agcctcttcacctttcgccgtatcgtgcatacctgtaaaaaggagttctcacggcggg | | |
| | | gcttgagtagccgaccatacagtatatacgacacatcgcgttaccgtatgtgagacgg | | |
| | | actaacgacgtagccgactacaagcgtacagacctttcataacactgtctgtcgcaga | | |
| | | agtccttaaagacgcatagacagtttacgaaggaaaaccttcaacccctgacaaa | | |
| | | aggggggagagagatcggatctttccatatcaagttatgacagaaagaccggaa | | |
| | | gcacggcgtactacagagcttgcaggtccaacccgtacgtcgtcacatgtatacg | | |
| | | agcagggaacttgtctacgctacgttttaagggcggatacagtattcagttactagg | | |
| | | catcccttaaacgtagtagtctataaggtcaagatttctagcgttaggtggacgttcg | | |
| | | cttacgtgccgtaaaacgcacaatattttacattaaaccttaagacctatgcgggcc | | |
| | | catcgttattcgcagacgaggagcgaataatgaaaacttttcaagtggaacccgg | | |
| | | acgccagtgctcgcaatttgagaagttggcgacacagcatttctgctgcgaaacg | | |
| | | atctagtaactggttcctcaacaaaggagcctgtcacaggtcgtttcgactacacca | | |
| | | tcgacagacacacatacggta | | |
| SEQ ID | Compl- | ttaaaacgaatccgattcggcgacagtagcctccgaatcatcatcgattaaattattc | DNA | Syn* |
| NO: 12 | imentary | tttttaaaactcattcctccaaaatccttaacatttctatagttcttgttcggcactg | | |
| | strand of | accgatcactactactatttttccctttgcggacatcactcttttttccggttcgaga | | |
| | TMV MP | tcgaaactttgcaagcctgatcgacatagggacatcttccatgaactcatcaacgact | | |
| | poly- | tcttctgtaagttccatgggccctccgtctctcacgtttgtaatcttctctctcaaac | | |
| | nucleotide | ctaattttatattatttctataaacaatacacaccgacacaaactccagagaaagcgg | | |
| | (5′-3′) | acagaaacccgctgacatcttcacatttctaatattaactaaaacttgccagacgttt | | |
| | | ttcatcgcgtcctgggtggttatagcataattgggaacgaccttgaactgaaatcttt | | |
| | | tctttgcagctgctgtgtagtaagatccgagagtggcctcgtcggctctttccatcct | | |
| | | tttgtccaccagacacacgctcacacctcctctgcaattgtcaggcaagttccactcg | | |
| | | cccgtgacgaccaaaccggctaaacagacgtatccactatcaataagcttaactcctt | | |
| | | taagaaggttcacccctgacaatgactcattctcatgaaccattattttatcaacttt | | |
| | | ggaacacataacactctttacaggggtaaacatcgacggtaagatcttctccattttt | | |
| | | gtcaggtcgataaactcattgatattcacttttcctttaacaactagagccat | | |
| SEQ ID | Comp- | TTATTGAGCAGAATCAGAATCATCTTCTTCATCAG | DNA | Syn* |
| NO: 13 | limentary | AAACAACAACATCTTGAGTACCAGAATCAAGAAC | | |
| | strand of | ATTAAGATAATTAGTAGGAAGAATATCACCAAGT | | |
| | CiLV-C- | TCATGAATTTGATTAATTTGTTGTCTAGCAGCAAG | | |
| | poly- | AAGAGCAGCAAGTTGAGCTTCAGCATAATCAGAA | | |
| | nucleotide | ACAGAATCCTTAGGAACATTCTTAACAATTTCAG | | |
| | (5′-3′) | CATTAGAACCAGGAAAAAAAACAACAATATAAT | | |
| | | GATCAGAAAATTCAAAAACAGGAACAACGTTCTT | | |
| | | AATAGGTCTCTTAGGAAAACCCATAGAATAAAGT | | |
| | | CTCTTTTCAAAAGAACCCATAGGATCCTTTTCAGA | | |
| | | AACAGCAACAAGATCTTCAGAAGCTTCCATAACC | | |
| | | TTAAAATCAGGAAGAGCGTAAGGTTCAGAAATCT | | |
| | | TATAATAAGTCATACCAATTTGCTTTTGAAGACCC | | |
| | | TTAGCTTCAGCATACTTAGGACAATGTTGTTCAA | | |
| | | GACCATAAAGCTTAGCCTTAGCAGCCTTCTTATTA | | |
| | | ATAGGAGGAGCAAGTTGCTTTTGCTTAGGCTTAA | | |
| | | CAACTTCAGCCTTTTGCTTAGCAGGCTTCTTAGCT | | |
| | | TGATAACCAGAAGGACCAGGTTGAAGTTGTTGAG | | |
| | | GTCTAGCAGAAGCTCTAGAAAGTTGTTGAGGTTG | | |
| | | AGATTGTTGCTTCTTCTTAGTAGGCTTAGAAATAT | | |
| | | CTCTAAGACCTTCAGGAACAGAAACGTTATGTCT | | |
| | | TTCAATAGCTTGCATAATTTCAGCAATAAGAGCA | | |
| | | GAAGAAGGATCAGTAAGAGTAAAAGTAACAATA | | |
| | | GACAT | | |
| SEQ ID | trfA- | ctagcgtttgcaatgcaccaggtcatcattgacccaggcgtgttccaccaggccg | DNA | Syn* |
| NO: 14 | poly- | ctgcctcgcaactcttcgcaggcttcgccgacctgctcgcgccacttcttcacgcg | | |
| | nucleotide | ggtggaatccgatccgcacatgaggcggaaggtttccagcttgagcgggtacgg | | |
| | | ctcccggtgcgagctgaaatagtcgaacatccgtcgggccgtcggcgacagctt | | |
| | | gcggtacttctcccatatgaatttcgtgtagtggtcgccagcaaacagcacgacga | | |
| | | tttcctcgtcgatcaggacctggcaacgggacgttttcttgccacggtccaggacg | | |
| | | cggaagcggtgcagcagcgacaccgattccaggtgcccaacgcggtcggacgt | | |
| | | ggagcccatcgccgtcgcctgtaggcgcgacaggcattcctcggccttcgtgtaa | | |
| | | taccggccattgatcgaccagcccaggtcctggcaaagctcgtagaacgtgaag | | |
| | | gtgatcggctcgccgataggggtgcgcttcgcgtactccaacacctgctgccaca | | |
| | | ccagttcgtcatcgtcggcccgcagctcgacgccggtgtaggtgatcttcacgtc | | |
| | | cttgttgacgtggaaaatgaccttgttttgcagcgcctcgcgcgggattttcttgttg | | |
| | | cgcgtggtgaacagggcagagcgggccgtgtcgtttggcatcgctcgcatcgtg | | |
| | | tccggccacggcgcaatatcgaacaaggaaagctgcatttccttgatctgctgctt | | |
| | | cgtgtgtttcagcaacgcggcctgcttggcctcgctgacctgttttgccaggtcctc | | |
| | | gccggcggtttttcgcttcttggtcgtcatagttcctcgcgtgtcgatggtcatcgac | | |
| | | ttcgccaaacctgccgcctcctgttcgagacgacgcgaacgctccacggcggcc | | |
| | | gatggcgcgggcagggcagggggagccagttgcacgctgtcgcgctcgatctt | | |
| | | ggccgtagcttgctgggccatcgagccgacggactggaaggtttcgcggggcg | | |
| | | cacgcatgacggtgcggcttgcgatggtttcggcatcctcggcggaaaaccccg | | |
| | | cgtcgatcagttcttgcctgtatgccttccggtcaaacgtccgattcat | | |
| SEQ ID | Comp- | atgaatcggacgtttgaccggaaggcatacaggcaagaactgatcgacgcggg | DNA | Syn* |
| NO: 15 | limentary | gttttccgccgaggatgccgaaaccatcgcaagccgcaccgtcatgcgtgcgcc | | |
| | strand of | ccgcgaaaccttccagtccgtcggctcgatggcccagcaagctacggccaagat | | |
| | trfA- | cgagcgcgacagcgtgcaactggctccccctgccctgcccgcgccatcggccg | | |
| | poly- | ccgtggagcgttcgcgtcgtctcgaacaggaggcggcaggtttggcgaagtcga | | |
| | nucleotide | tgaccatcgacacgcgaggaactatgacgaccaagaagcgaaaaaccgccgg | | |
| | (5′-3′) | cgaggacctggcaaaacaggtcagcgaggccaagcaggccgcgttgctgaaa | | |
| | | cacacgaagcagcagatcaaggaaatgcagctttccttgttcgatattgcgccgtg | | |
| | | gccggacacgatgcgagcgatgccaaacgacacggcccgctctgccctgttca | | |
| | | ccacgcgcaacaagaaaatcccgcgcgaggcgctgcaaaacaaggtcattttcc | | |
| | | acgtcaacaaggacgtgaagatcacctacaccggcgtcgagctgcgggccgac | | |
| | | gatgacgaactggtgtggcagcaggtgttggagtacgcgaagcgcacccctatc | | |
| | | ggcgagccgatcaccttcacgttctacgagctttgccaggacctgggctggtcga | | |
| | | tcaatggccggtattacacgaaggccgaggaatgcctgtcgcgcctacaggcga | | |
| | | cggcgatgggctccacgtccgaccgcgttgggcacctggaatcggtgtcgctgc | | |
| | | tgcaccgcttccgcgtcctggaccgtggcaagaaaacgtcccgttgccaggtcct | | |
| | | gatcgacgaggaaatcgtcgtgctgtttgctggcgaccactacacgaaattcatat | | |
| | | gggagaagtaccgcaagctgtcgccgacggcccgacggatgttcgactatttca | | |
| | | gctcgcaccgggagccgtacccgctcaagctggaaaccttccgcctcatgtgcg | | |
| | | gatcggattccacccgcgtgaagaagtggcgcgagcaggtcggcgaagcctgc | | |
| | | gaagagttgcgaggcagcggcctggtggaacacgcctgggtcaatgatgacct | | |
| | | ggtgcattgcaaacgctag | | |
| SEQ ID | trfA- | “MNRTFDRKAYRQELIDAGFSAEDAETIASRTVMRA | AA | Syn* |
| NO: 16 | poly- | PRETFQSVGSMAQQATAKIERDSVQLAPPALPAPSA | | |
| | peptide | AVERSRRLEQEAAGLAKSMTIDTRGTMTTKKRKTA | | |
| | | GEDLAKQVSEAKQAALLKHTKQQIKEMQLSLFDIA | | |
| | | PWPDTMRAMPNDTARSALFTTRNKKIPREALQNKV | | |
| | | IFHVNKDVKITYTGVELRADDDELVWQQVLEYAKR | | |
| | | TPIGEPITFTFYELCQDLGWSINGRYYTKAEECLSRL | | |
| | | QATAMGFTSDRVGHLESVSLLHRFRVLDRGKKTSR | | |
| | | CQVLIDEEIVVLFAGDHYTKFIWEK | | |
| | | YRKLSPTARRMFDYFSSHREPYPLKLETFRLMCGSD | | |
| | | STRVKKWREQVGEACEELRGSGLVEHAWVNDDLV | | |
| | | HCKR” | | |
| SEQ ID | KanR- | “ctaaaacaattcatccagtaaaatataatattttattttctcccaatcaggcttgat | DNA | Syn* |
| NO: 17 | poly- | ccccagtaagtcaaaaaatagctcgacatactgttcttccccgatatcctccctgatc | | |
| | nucleotide | gaccggacgcagaaggcaatgtcataccacttgtccgccctgccgcttctcccaag | | |
| | | atcaataaagccacttactttgccatctttcacaaagatgttgctgtctcccaggtcg | | |
| | | ccgtgggaaaagacaagttcctcttcgggcttttccgtctttaaaaaatcatacagct | | |
| | | cgcgcggatctttaaatggagtgtcttcttcccagttttcgcaatccacatcggcca | | |
| | | gatcgttattcagtaagtaatccaattcggctaagcggctgtctaagctattcgtata | | |
| | | gggacaatccgatatgtcgatggagtgaaagagcctgatgcactccgcatacag | | |
| | | ctcgataatcttttcagggctttgttcatcttcatactcttccgagcaaaggacgcca | | |
| | | tcggcctcactcatgagcagattgctccagccatcatgccgttcaaagtgcaggac | | |
| | | ctttggaacaggcagctttccttccagccatagcatcatgtccttttcccgttccaca | | |
| | | tcataggtggtccctttataccggctgtccgtcatttttaaatataggttttcatttt | | |
| | | ctcccaccagcttatataccttagcaggagacattccttccgtatcttttacgcagcg | | |
| | | gtatttttcgatcagttttttcaattccggtgatattctcattttagccat” | | |
| SEQ ID | Comp- | atggctaaaatgagaatatcaccggaattgaaaaaactgatcgaaaaataccgct | DNA | Syn* |
| NO: 18 | limentary | gcgtaaaagatacggaaggaatgtctcctgctaaggtatataagctggtgggaga | | |
| | strand of | aaatgaaaacctatatttaaaaatgacggacagccggtataaagggaccacctat | | |
| | KanR- | gatgtggaacgggaaaaggacatgatgctatggctggaaggaaagctgcctgtt | | |
| | poly- | ccaaaggtcctgcactttgaacggcatgatggctggagcaatctgctcatgagtg | | |
| | nucleotide | aggccgatggcgtcctttgctcggaagagtatgaagatgaacaaagccctgaaa | | |
| | (5′-3′) | agattatcgagctgtatgcggagtgcatcaggctctttcactccatcgacatatcgg | | |
| | | attgtccctatacgaatagcttagacagccgcttagccgaattggattacttactgaa | | |
| | | taacgatctggccgatgtggattgcgaaaactgggaagaagacactccatttaaa | | |
| | | gatccgcgcgagctgtatgattttttaaagacggaaaagcccgaagaggaacttg | | |
| | | tcttttcccacggcgacctgggagacagcaacatctttgtgaaagatggcaaagta | | |
| | | agtggctttattgatcttgggagaagcggcagggcggacaagtggtatgacattg | | |
| | | ccttctgcgtccggtcgatcagggaggatatcggggaagaacagtatgtcgagct | | |
| | | attttttgacttactggggatcaagcctgattgggagaaaataaaatattatatttta | | |
| | | ctggatgaattgttttag | | |
| SEQ ID | KanR- | “FLEDLLIYYKIKEWDPKIGLLDFFLEVYQEEGIDERI | AA | Syn* |
| NO: 19 | poly- | SRVCFAIDYWKDARGSRGLDIFGSVKGDKVFINSDG | | |
| | peptide | LDGSFVLEEEPKETKLFDYLRPDKFPTDEEWNECyc | | |
| | | DVDALDNNLLYDLEALRSDLSNTYPCycDSIDISHFL | | |
| | | RICEAYLEIKEPSQEDEYEESCLVGDAESMLLNSWG | | |
| | | DHREFHLVKPVPLKGELWLMMDKEREVDYTTGKY | | |
| | | RSDTMKLYLNENEGVLKYVAKPSM | | |
| | | GETDKVCRYKEILKKLEPSIRMKAM” | | |
| SEQ ID | oriV (a) | “agcgggccgggagggttcgagaagggggggcaccccccttcggcgtgcgc | DNA | Syn* |
| NO: 20 | | ggtcacgcgcacagggcgcagccctggttaaaaacaaggtttataaatattggttt | | |
| | | aaaagcaggttaaaagacaggttagcggtggccgaaaaacgggcggaaaccct | | |
| | | tgcaaatgctggattttctgcctgtggacagcccctcaaatgtcaataggtgcgccc | | |
| | | ctcatctgtcagcactctgcccctcaagtgtcaaggatcgcgcccctcatctgtcag | | |
| | | tagtcgcgcccctcaagtgtcaataccgcagggcacttatccccaggcttgtccac | | |
| | | atcatctgtgggaaactcgcgtaaaatcaggcgttttcgccgatttgcgaggctgg | | |
| | | ccagctccacgtcgccggccgaaatcgagcctgcccctcatctgtcaacgccgc | | |
| | | gccgggtgagtcggcccctcaagtgtcaacgtccgcccctcatctgtcagtgagg | | |
| | | gccaagttttccgcgaggtatccacaacgccggcggcc” | | |
| SEQ ID | Comp- | ggccgccggcgttgtggatacctcgcggaaaacttggccctcactgacagatga | DNA | Syn* |
| NO: 21 | limentary | ggggcggacgttgacacttgaggggccgactcacccggcgcggcgttgacag | | |
| | strand of | atgaggggcaggctcgatttcggccggcgacgtggagctggccagcctcgcaa | | |
| | oriV (a) | atcggcgaaaacgcctgattttacgcgagtttcccacagatgatgtggacaagcct | | |
| | | ggggataagtgccctgcggtattgacacttgaggggcgcgactactgacagatg | | |
| | | aggggcgcgatccttgacacttgaggggcagagtgctgacagatgaggggcgc | | |
| | | acctattgacatttgaggggctgtccacaggcagaaaatccagcatttgcaagggt | | |
| | | ttccgcccgtttttcggccaccgctaacctgtcttttaacctgcttttaaaccaatat | | |
| | | ttataaaccttgtttttaaccagggctgcgccctgtgcgcgtgaccgcgcacgccga | | |
| | | aggggggtgcccccccttctcgaaccctcccggcccgct | | |
| SEQ ID | oriV (b) | “gggagggttcgagaagggggggcaccccccttcggcgtgcgcggtcacgcg | DNA | Syn* |
| NO: 22 | | cacagggcgcagccctggttaaaaacaaggtttataaatattggtttaaaagcagg | | |
| | | ttaaaagacaggttagcggtggccgaaaaacgggcggaaacccttgcaaatgct | | |
| | | ggattttctgcctgtggacagcccctcaaatgtcaataggtgcgcccctcatctgtc | | |
| | | agcactctgcccctcaagtgtcaaggatcgcgcccctcatctgtcagtagtcgcgc | | |
| | | ccctcaagtgtcaataccgcagggcacttatccccaggcttgtccacatcatctgtg | | |
| | | ggaaactcgcgtaaaatcaggcgttttcgccgatttgcgaggctggccagctcca | | |
| | | cgtcgccggccgaaatcgagcctgcccctcatctgtcaacgccgcgccgggtga | | |
| | | gtcggcccctcaagtgtcaacgtccgcccctcatctgtcagtgagggccaagtttt | | |
| | | ccgcgaggtatccacaacgccggcggccggccgcggtgtctcgcacacggctt | | |
| | | cgacggcgtttctggcgcgtttgcagggccatagacggccgccagcccagcgg | | |
| | | cgagggcaaccag” | | |
| SEQ ID | Comp- | ctggttgccctcgccgctgggctggcggccgtctatggccctgcaaacgcgcca | DNA | Syn* |
| NO: 23 | limentary | gaaacgccgtcgaagccgtgtgcgagacaccgcggccggccgccggcgttgt | | |
| | strand of | ggatacctcgcggaaaacttggccctcactgacagatgaggggcggacgttgac | | |
| | oriV (b) | acttgaggggccgactcacccggcgcggcgttgacagatgaggggcaggctc | | |
| | | gatttcggccggcgacgtggagctggccagcctcgcaaatcggcgaaaacgcc | | |
| | | tgattttacgcgagtttcccacagatgatgtggacaagcctggggataagtgccct | | |
| | | gcggtattgacacttgaggggcgcgactactgacagatgaggggcgcgatcctt | | |
| | | gacacttgaggggcagagtgctgacagatgaggggcgcacctattgacatttga | | |
| | | ggggctgtccacaggcagaaaatccagcatttgcaagggtttccgcccgtttttcg | | |
| | | gccaccgctaacctgtcttttaacctgcttttaaaccaatatttataaaccttgtttt | | |
| | | taaccagggctgcgccctgtgcgcgtgaccgcgcacgccgaaggggggtgccccc | | |
| | | ccttctcgaaccctccc | | |
| SEQ ID | LB T- | gtggcaggatatattgtggtgtaaac | DNA | Syn* |
| NO: 24 | DNA | | | |
| | repeat | | | |
| SEQ ID | Comp- | gtttacaccacaatatatcctgccacc | DNA | Syn* |
| NO: 25 | limentary | | | |
| | strand of | | | |
| | LB T- | | | |
| | DNA | | | |
| | repeat | | | |
| SEQ ID | KS primer | cgaggtcgacggtatcg | DNA | Syn* |
| NO: 26 | | | | |
| SEQ ID | Comp- | cgataccgtcgacctcg | DNA | Syn* |
| NO: 27 | limentary | | | |
| | strand of | | | |
| | KS primer | | | |
| SEQ ID | minimal | “gcaagaccttcctctatataaggaagttcatttcatttggagagg” | DNA | Syn* |
| NO: 28 | CaMV | | | |
| | 35S | | | |
| | promoter | | | |
| SEQ ID | Comp- | cctctccaaatgaaatgaacttccttatatagaggaaggtcttgc | DNA | Syn* |
| NO: 29 | limentary | | | |
| | strand of | | | |
| | minimal | | | |
| | CaMV | | | |
| | 35S | | | |
| | promoter | | | |
| SEQ ID | ″pTRBO_ | “gtatttttacaacaattaccaacaacaacaaacaacagacaacattacaattactat | DNA | Syn* |
| NO: 30 | p29_CiLV | ttacaattacaatggcatacacacagacagctaccacatcagctttgctggacact | | |
| | C” | gtccgaggaaacaactccttggtcaatgatctagcaaagcgtcgtctttacgacac | | |
| | | agcggttgaagagtttaacgctcgtgaccgcaggcccaaggtgaacttttcaaaa | | |
| | | gtaataagcgaggagcagacgcttattgctacccgggcgtatccagaattccaaa | | |
| | | ttacattttataacacgcaaaatgccgtgcattcgcttgcaggtggattgcgatcttt | | |
| | | agaactggaatatctgatgatgcaaattccctacggatcattgacttatgacataggc | | |
| | | gggaattttgcatcgcatctgttcaagggacgagcatatgtacactgctgcatgcc | | |
| | | caacctggacgttcgagacatcatgcggcacgaaggccagaaagacagtattga | | |
| | | actatacctttctaggctagagagaggggggaaaacagtccccaacttccaaaag | | |
| | | gaagcatttgacagatacgcagaaattcctgaagacgctgtctgtcacaatactttc | | |
| | | cagacatgcgaacatcagccgatgcagcaatcaggcagagtgtatgccattgcg | | |
| | | ctacacagcatatatgacataccagccgatgagttcggggcggcactcttgagga | | |
| | | aaaatgtccatacgtgctatgccgctttccacttctccgagaacctgcttcttgaaga | | |
| | | ttcatgcgtcaatttggacgaaatcaacgcgtgtttttcgcgcgatggagacaagtt | | |
| | | gaccttttcttttgcatcagagagtactcttaattactgtcatagttattctaatatt | | |
| | | cttaagtatgtgtgcaaaacttacttcccggcctctaatagagaggtttacatgaagg | | |
| | | agtttttagtcaccagagttaatacctggttttgtaagttttctagaatagatacttt | | |
| | | tcttttgtacaaaggtgtggcccataaaagtgtagatagtgagcagttttatactgca | | |
| | | atggaagacgcatggcattacaaaaagactcttgcaatgtgcaacagcgagagaatcc | | |
| | | tccttgaggattcatcatcagtcaattactggtttcccaaaatgagggatatggtcat | | |
| | | cgtaccattattcgacatttctttggagactagtaagaggacgcgcaaggaagtctta | | |
| | | gtgtccaaggatttcgtgtttacagtgcttaaccacattcgaacataccaggcgaaag | | |
| | | ctcttacatacgcaaatgttttgtccttcgtcgaatcgattcgatcgagggtaatcat | | |
| | | taacggtgtgacagcgaggtccgaatgggatgtggacaaatctttgttacaatcctt | | |
| | | gtccatgacgttttacctgcatactaagcttgccgttctaaaggatgacttactgatt | | |
| | | agcaagtttagtctcggttcgaaaacggtgtgccagcatgtgtgggatgagatttcg | | |
| | | ctggcgtttgggaacgcatttccctccgtgaaagagaggctcttgaacaggaaac | | |
| | | ttatcagagtggcaggcgacgcattagagatcagggtgcctgatctatatgtgacc | | |
| | | ttcc | | |
| | | acgacagattagtgactgagtacaaggcctctgtggacatgcctgcgcttgacatt | | |
| | | aggaagaagatggaagaaacggaagtgatgtacaatgcactttcagaattatcgg | | |
| | | tgttaagggagtctgacaaattcgatgttgatgttttttcccagatgtgccaatcttt | | |
| | | ggaagttgacccaatgacggcagcgaaggttatagtcgcggtcatgagcaatgag | | |
| | | agcggtctgactctcacatttgaacgacctactgaggcgaatgttgcgctagcttta | | |
| | | caggatcaagagaaggcttcagaaggtgcattggtagttacctcaagagaagttg | | |
| | | aagaaccgtccatgaagggttcgatggccagaggagagttacaattagctggtct | | |
| | | tgctggagatcatccggaatcgtcctattctaagaacgaggagatagagtctttag | | |
| | | agcagtttcatatggcgacggcagattcgttaattcgtaagcagatgagctcgattg | | |
| | | tgtacacgggtccgattaaagttcagcaaatgaaaaactttatcgatagcctggtag | | |
| | | catcactatctgctgcggtgtcgaatctcgtcaagatcctcaaagatacagctgcta | | |
| | | ttgaccttgaaacccgtcaaaagtttggagtcttggatgttgcatctaggaagtggtt | | |
| | | aatcaaaccaacggccaagagtcatgcatggggtgttgttgaaacccacgcgag | | |
| | | gaagtatcatgtggcgcttttggaatatgatgagcagggtgtggtgacatgcgatg | | |
| | | attggagaagagtagctgttagctctgagtctgttgtttattccgacatggcgaaact | | |
| | | cagaactctgcgcagactgcttcgaaacggagaaccgcatgtcagtagcgcaaa | | |
| | | ggttgttcttgtggacggagttccgggctgtggaaaaaccaaagaaattctttcca | | |
| | | gggttaattttgatgaagatctaattttagtacctgggaagcaagccgcggaaatga | | |
| | | tcagaagacgtgcgaattcctcagggattattgtggccacgaaggacaacgttaa | | |
| | | aaccgttgattctttcatgatgaattttgggaaaagcacacgctgtcagttcaagag | | |
| | | gttattcattgatgaagggttgatgttgcatactggttgtgttaattttcttgtggcg | | |
| | | atgtcattgtgcgaaattgcatatgtttacggagacacacagcagattccatacatca | | |
| | | atagagtttcaggattcccgtaccccgcccattttgccaaattggaagttgacgaggt | | |
| | | ggagacacgcagaactactctccgttgtccagccgatgtcacacattatctgaaca | | |
| | | ggagatatgagggctttgtcatgagcacttcttcggttaaaaagtctgtttcgcagg | | |
| | | agatggtcggcggagccgccgtgatcaatccgatctcaaaacccttgcatggcaa | | |
| | | gatcttgacttttacccaatcggataaagaagctctgctttcaagagggtattcagat | | |
| | | gttcacactgtgcatgaagtgcaaggcgagacatactctgatgtttcactagttagg | | |
| | | ttaacccctacaccggtctccatcattgcaggagacagcccacatgttttggtcgca | | |
| | | ttgtcaaggcacacctgttcgctcaagtactacactgttgttatggatcctttagtta | | |
| | | gtatcattagagatctagagaaacttagctcgtacttgttagatatgtataaggtcga | | |
| | | tgcaggaacacaatagcaattacagattgactcggtgttcaaaggttccaatcttttt | | |
| | | gttgcagcgccaaagactggtgatatttctgatatgcagttttactatgataagtgtc | | |
| | | tcccaggcaacagcaccatgatgaataattttgatgctgttaccatgaggttgactga | | |
| | | catttcattgaatgtcaaagattgcatattggatatgtctaagtctgttgctgcgcct | | |
| | | aaggatcaaatcaaaccactaatacctatggtacgaacggcggcagaaatgccacg | | |
| | | ccagactggactattggaaaatttagtggcgatgattaaaagaaactttaacgcac | | |
| | | ccgagttgtctggcatcattgatattgaaaatactgcatctttggttgtagataagtt | | |
| | | ttttgatagttatttgcttaaagaaaaaagaaaaccaaataaaaatgtttctttgttc | | |
| | | agtagagagtctctcaatagatggttagaaaagcaggaacaggtaacaataggccagc | | |
| | | tcgcagattttgattttgtggatttgccagcagttgatcagtacagacacatgattaa | | |
| | | agcacaacccaaacaaaagttggacacttcaatccaaacggagtacccggctttgc | | |
| | | agacgattgtgtaccattcaaaaaagatcaatgcaatattcggcccgttgtttagtga | | |
| | | gcttactaggcaattactggacagtgttgattcgagcagatttttgtttttcacaaga | | |
| | | aagacaccagcgcagattgaggatttcttcggagatctcgacagtcatgtgccgat | | |
| | | ggatgtcttggagctggatatatcaaaatacgacaaatctcagaatgaattccactg | | |
| | | tgcagtagaatacgagatctggcgaagattgggtttcgaagacttcttgggagaa | | |
| | | gtttggaaacaagggcatagaaagaccaccctcaaggattataccgcaggtataa | | |
| | | aaacttgcatctggtatcaaagaaagagcggggacgtcacgacgttcattggaaa | | |
| | | cactgtgatcattgctgcatgtttggcctcgatgcttccgatggagaaaataatcaa | | |
| | | aggagccttttgcggtgacgatagtctgctgtactttccaaagggttgtgagtttcc | | |
| | | ggatgtgcaacactccgcgaatcttatgtggaattttgaagcaaaactgtttaaaaa | | |
| | | acagtatggatacttttgcggaagatatgtaatacatcacgacagaggatgcattgt | | |
| | | gtattacgatcccctaaagttgatctcgaaacttggtgctaaacacatcaaggattg | | |
| | | ggaacacttggaggagttcagaaggtctctttgtgatgttgctgtttcgttgaacaat | | |
| | | tgtgcgtattacacacagttggacgacgctgtatgggaggttcataagaccgccc | | |
| | | ctccaggttcgtttgtttataaaagtctggtgaagtatttgtctgataaagttctttt | | |
| | | tagaagtttgtttatagatggctctagttgttaa | | |
| | | aggaaaagtgaatatcaatgagtttatcgacctgacaaaaatggagaagatcttac | | |
| | | cgtcgatgtttacccctgtaaagagtgttatgtgttccaaagttgataaaataatggt | | |
| | | tcatgagaatgagtcattgtcaggggtgaaccttcttaaaggagttaagcttattgat | | |
| | | agtggatacgtctgtttagccggtttggtcgtcacgggcgagtggaacttgcctga | | |
| | | caattgcagaggaggtgtgagcgtgtgtctggtggacaaaaggatggaaagagc | | |
| | | cgacgaggccactctcggatcttactacacagcagctgcaaagaaaagatttcag | | |
| | | ttcaaggtcgttcccaattatgctataaccacccaggacgcgatgaaaaacgtctg | | |
| | | gcaagttttagttaatattagaaatgtgaagatgtcagcgggtttctgtccgctttct | | |
| | | ctggagtttgtgtcggtgtgtattgtttatagaaataatataaaattaggtttgagag | | |
| | | agaagattacaaacgtgagagacggagggcccatggaacttacagaagaagtcgtt | | |
| | | gatgagttcatggaagatgtccctatgtcgatcaggcttgcaaagtttcgatctcga | | |
| | | accggaaaaaagagtgatgtccgcaaagggaaaaatagtagtagtgatcggtca | | |
| | | gtgccgaacaagaactatagaaatgttaaggattttggaggaatgagttttaaaaa | | |
| | | gaataatttaatcgatgatgattcggaggctactgtcgccgaatcggattcgttttaa | | |
| | | atagatcttac | | |
| | | agtatcactactccatctcagttcgtgttcttgtcattaatTAAGGCGCGCC | | |
| | | ATGTCTATTGTTACTTTTACTCTTACTGATCCTTCT | | |
| | | TCTGCTCTTATTGCTGAAATTATGCAAGCTATTGA | | |
| | | AAGACATAACGTTTCTGTTCCTGAAGGTCTTAGA | | |
| | | GATATTTCTAAGCCTACTAAGAAGAAGCAACAAT | | |
| | | CTCAACCTCAACAACTTTCTAGAGCTTCTGCTAGA | | |
| | | CCTCAACAACTTCAACCTGGTCCTTCTGGTTATCA | | |
| | | AGCTAAGAAGCCTGCTAAGCAAAAGGCTGAAGTT | | |
| | | GTTAAGCCTAAGCAAAAGCAACTTGCTCCTCCTA | | |
| | | TTAATAAGAAGGCTGCTAAGGCTAAGCTTTATGG | | |
| | | TCTTGAACAACATTGTCCTAAGTATGCTGAAGCT | | |
| | | AAGGGTCTTCAAAAGCAAATTGGTATGACTTATT | | |
| | | ATAAGATTTCTGAACCTTACGCTCTTCCTGATTTT | | |
| | | AAGGTTATGGAAGCTTCTGAAGATCTTGTTGCTG | | |
| | | TTTCTGAAAAGGATCCTATGGGTTCTTTTGAAAA | | |
| | | GAGACTTTATTCTATGGGTTTTCCTAAGAGACCTA | | |
| | | TTAAGAACGTTGTTCCTGTTTTTGAATTTTCTGAT | | |
| | | CATTATATTGTTGTTTTTTTTCCTGGTTCTAATGCT | | |
| | | GAAATTGTTAAGAATGTTCCTAAGGATTCTGTTTC | | |
| | | TGATTATGCTGAAGCTCAACTTGCTGCTCTTCTTG | | |
| | | CTGCTAGACAACAAATTAATCAAATTCATGAACT | | |
| | | TGGTGATATTCTTCCTACTAATTATCTTAATGTTC | | |
| | | TTGATTCTGGTACTCAAGATGTTGTTGTTTCTGAT | | |
| | | GAAGAAGATGATTCTGATTCTGCTCAATAA | | |
| | | TAGCCCGGGCctagggcggccgcggtcctgcaactt | | |
| | | Gaggtagtcaagatgcataataaataacggattgtgtccgtaatcacacgtggtg | | |
| | | Cgtacgataacgcatagtgtttttccctccacttaaatcgaagggttgtgtcttg | | |
| | | gatcgcgcgggtcaaatgtatatggttcatatacatccgcaggcacgtaataaag | | |
| | | Cgaggggttcgaatccccccgttacccccggtaggggcccaggtacccggatg | | |
| | | tg | | |
| | | Ttttccgggttgatgagtccgtgaggacgaaacctggctgcaggcatgcaagctt | | |
| | | Ggcgtaatcatggtcatagcctagagtccgcaaaaatcaccagtctctctctaca | | |
| | | Aatctatctctctctatttttctccagaataatgtgtgagtagttcccagataag | | |
| | | Ggaattagggttcttatagggtttcgctcatgtgttgagcatataagaaaccctt | | |
| | | Agtatgtatttgtatttgtaaaatacttctatcaataaaatttctaattcctaaa | | |
| | | Accaaaatccagtgacctgcagcccggccgggggatccactagcagattgtcgt | | |
| | | t Tcccgccttcagtttaaactatcagtgtttgacaggatatattgggggtaaacc | | |
| | | Taagagaaaagagcgtttattagaataatcggatatttaaaagggcgtgaaaagg | | |
| | | Tttatccgttcgtccatttgtatgtgcatgccaaccacaggagatctcagtaaag | | |
| | | Cgctggctgaacccccagccggaactgaccccacaaggcc | | |
| | | Ctagcgtttgcaatgcaccaggtcatcattgacccaggcgtgttccaccaggccg | | |
| | | ctgcctcgcaactcttcgcaggcttcgccgacctgctcgcgccacttcttcacgcg | | |
| | | ggtggaatccgatccgcacatgaggcggaaggtttccagcttgagcgggtacgg | | |
| | | ctcccggtgcgagctgaaatagtcgaacatccgtcgggccgtcggcgacagctt | | |
| | | gcggtacttctcccatatgaatttcgtgtagtggtcgccagcaaacagcacgacga | | |
| | | tttcctcgtcgatcaggacctggcaacgggacgttttcttgccacggtccaggacg | | |
| | | cggaagcggtgcagcagcgacaccgattccaggtgcccaacgcggtcggacgt | | |
| | | ggagcccatcgccgtcgcctgtaggcgcgacaggcattcctcggccttcgtgtaa | | |
| | | taccggccattgatcgaccagcccaggtcctggcaaagctcgtagaacgtgaag | | |
| | | gtgatcggctcgccgataggggtgcgcttcgcgtactccaacacctgctgccaca | | |
| | | ccagttcgtcatcgtcggcccgcagctcgacgccggtgtaggtgatcttcacgtc | | |
| | | cttgttgacgtggaaaatgaccttgttttgcagcgcctcgcgcgggattttcttgttg | | |
| | | cgcgtggtgaacagggcagagcgggccgtgtcgtttggcatcgctcgcatcgtg | | |
| | | tccggccacggcgcaatatcgaacaaggaaagctgcatttccttgatctgctgctt | | |
| | | cgtgtgtttcagcaacgcggcctgcttggcctcgctgacctgttttgccaggtcctc | | |
| | | gccggcggtttttcgcttcttggtcgtcatagttcctcgcgtgtcgatggtcatcgac | | |
| | | ttcgccaaacctgccgcctcctgttcgagacgacgcgaacgctccacggcggcc | | |
| | | gatggcgcgggcagggcagggggagccagttgcacgctgtcgcgctcgatctt | | |
| | | ggccgtagcttgctgggccatcgagccgacggactggaaggtttcgcggggcg | | |
| | | cacgcatgacggtgcggcttgcgatggtttcggcatcctcggcggaaaaccccg | | |
| | | cgtcgatcagttcttgcctgtatgccttccggtcaaacgtccgattcat | | |
| | | Tcaccctccttgcgggattgc | | |
| | | Cccgactcacgccggggcaatgtgcccttattcctgatttgacccgcctggtgcc | | |
| | | Ttggtgtccagataatccaccttatcggcaatgaagtcggtcccgtagaccgtct | | |
| | | Ggccgtccttctcgtacttggtattccgaatcttgccctgcacgaataccagcga | | |
| | | Ccccttgcccaaatacttgccgtgggcctcggcctgagagccaaaacacttgatg | | |
| | | Cggaagaagtcggtgcgctcctgcttgtcgccggcatcgttgcgccacatctag | | |
| | | g ta | | |
| | | ctaaaacaattcatccagtaaaatataatattttattttctcccaatcaggcttgatc | | |
| | | cccagtaagtcaaaaaatagctcgacatactgttcttccccgatatcctccctgatcg | | |
| | | accggacgcagaaggcaatgtcataccacttgtccgccctgccgcttctcccaaga | | |
| | | tcaataaagccacttactttgccatctttcacaaagatgttgctgtctcccaggtcgc | | |
| | | cgtgggaaaagacaagttcctcttcgggcttttccgtctttaaaaaatcatacagctc | | |
| | | gcgcggatctttaaatggagtgtcttcttcccagttttcgcaatccacatcggccag | | |
| | | atcgttattcagtaagtaatccaattcggctaagcggctgtctaagctattcgtatag | | |
| | | ggacaatccgatatgtcgatggagtgaaagagcctgatgcactccgcatacagct | | |
| | | cgataatcttttcagggctttgttcatcttcatactcttccgagcaaaggacgccatc | | |
| | | ggcctcactcatgagcagattgctccagccatcatgccgttcaaagtgcaggacct | | |
| | | ttggaacaggcagctttccttccagccatagcatcatgtccttttcccgttccacatc | | |
| | | ataggtggtccctttataccggctgtccgtcatttttaaatataggttttcattttct | | |
| | | cccaccagcttatataccttagcaggagacattccttccgtatcttttacgcagcggt | | |
| | | atttttcgatcagttttttcaattccggtgatattctcattttagccat | | |
| | | ttattatttccttcctcttttctacagt | | |
| | | atttaaagataccccaagaagctaattataacaagacgaactccaattcactgtt | | |
| | | ccttgcattctaaaaccttaaataccagaaaacagctttttcaaagttgttttca | | |
| | | aagttggcgtataacatagtatcgacggagccgattttgaaaccacaattatggg | | |
| | | tgatgctgccaactcgag | | |
| | | agcgggccgggagggttcgagaagggggggcaccccccttcggcgtgcgcg | | |
| | | gtcacgcgcacagggcgcagccctggttaaaaacaaggtttataaatattggttta | | |
| | | aaagcaggttaaaagacaggttagcggtggccgaaaaacgggcggaaaccctt | | |
| | | gcaaatgctggattttctgcctgtggacagcccctcaaatgtcaataggtgcgccc | | |
| | | ctcatctgtcagcactctgcccctcaagtgtcaaggatcgcgcccctcatctgtcag | | |
| | | tagtcgcgcccctcaagtgtcaataccgcagggcacttatccccaggcttgtccac | | |
| | | atcatctgtgggaaactcgcgtaaaatcaggcgttttcgccgatttgcgaggctgg | | |
| | | ccagctccacgtcgccggccgaaatcgagcctgcccctcatctgtcaacgccgc | | |
| | | gccgggtgagtcggcccctcaagtgtcaacgtccgcccctcatctgtcagtgagg | | |
| | | gccaagttttccgcgaggtatccacaacgccggcggcc | | |
| | | ggccgcggtgtctcgcacacggcttcgacggcgtttctggcgcgtttgcagggc | | |
| | | c | | |
| | | atagacggccgccagcccagcggcgagggcaaccagcccggtgagctctagt | | |
| | | gga | | |
| | | ctgatgggctgcctgtatcgagtggtgattttgtgccgagctgccggtcggggag | | |
| | | ctgttggctggctggtggcaggatatattgtggtgtaaacaaattgacgcttaga | | |
| | | caacttaataacacattgcggacgtttttaatgtactggggtggttttggtaccg | | |
| | | ggccccccctcgaggtcgacggtatcgataagcttgatatcgaattcctgcaggt | | |
| | | caacatggtggagcacgacactctcgtctactccaagaatatcaaagatacagtc | | |
| | | tcagaagaccaaagggctattgagacttttcaacaaagggtaatatcgggaaacc | | |
| | | tcctcggattccattgcccagctatctgtcacttcatcaaaaggacagtagaaaa | | |
| | | ggaaggtggcacctacaaatgccatcattgcgataaaggaaaggctatcgttcaa | | |
| | | gatgcctctgccgacagtggtcccaaagatggacccccacccacgaggagcatc | | |
| | | g | | |
| | | tggaaaaagaagacgttccaaccacgtcttcaaagcaagtggattgatgtgataa | | |
| | | catggtggagcacgacactctcgtctactccaagaatatcaaagatacagtctca | | |
| | | gaagaccaaagggcaattgagacttttcaacaaagggtaatatccggaaacctcc | | |
| | | tcggattccattgcccagctatctgtcactttattgtgaagatagtggaaaagga | | |
| | | aggtggctcctacaaatgccatcattgcgataaaggaaaggccatcgttgaagat | | |
| | | gcctctgccgacagtggtcccaaagatggacccccacccacgaggagcatcgt | | |
| | | gg | | |
| | | aaaaagaagacgttccaaccacgtcttcaaagcaagtggattgatgtgatatctc | | |
| | | cactgacgtaagggatgacgcacaatcccactatccttcgcaagaccttcctcta | | |
| | | tataaggaagttcatttcatttggagagg” | | |
| SEQ ID | “Comp- | cctctccaaatgaaatgaacttccttatatagaggaaggtcttgcgaaggatagtgg | DNA | Syn* |
| NO: 31 | limentary | gattgtgcgtcatcccttacgtcagtggagatatcacatcaatccacttgctttgaag | | |
| | strand of | acgtggttggaacgtcttctttttccacgatgctcctcgtggggggggtccatcttt | | |
| | pTRBO_ | gggaccactgtcggcagaggcatcttcaacgatggcctttcctttatcgcaatgat | | |
| | p29_ | ggcatttgtaggagccaccttccttttccactatcttcacaataaagtgacagatagc | | |
| | CiLVC” | tgggcaatggaatccgaggaggtttccggatattaccctttgttgaaaagtctcaat | | |
| | | tgccctttggtcttctgagactgtatctttgatattcttggagtagacgagagtgtcg | | |
| | | tgctccaccatgttatcacatcaatccacttgctttgaagacgtggttggaacgtctt | | |
| | | ctttttccacgatgctcctcgtgggtgggggtccatctttgggaccactgtcggcaga | | |
| | | ggcatcttgaacgatagcctttcctttatcgcaatgatggcatttgtaggtgccacct | | |
| | | tccttttctactgtccttttgatgaagtgacagatagctgggcaatggaatccgagg | | |
| | | aggtttcccgatattaccctttgttgaaaagtctcaatagccctttggtcttctgaga | | |
| | | tgtatctttgatattcttggagtagacgagagtgtcgtgctccaccatgttgacctgc | | |
| | | caggaattcgatatcaagcttatcgataccgtcgacctcgagggggggcccggtac | | |
| | | caaaaccaccccagtacattaaaaacgtccgcaatgtgttattaagttgtctaagcg | | |
| | | tcaatttgtttacaccacaatatatcctgccaccagccagccaacagctccccgac | | |
| | | cggcagctcggcacaaaatcaccactcgatacaggcagcccatcagtccactag | | |
| | | agctcaccgggctggttgccctcgccgctgggctggcggccgtctatggccctg | | |
| | | caaacgcgccagaaacgccgtcgaagccgtgtgcgagacaccgcggccggcc | | |
| | | gccggcgttgtggatacctcgcggaaaacttggccctcactgacagatgagggg | | |
| | | cggacgttgacacttgaggggccgactcacccggcgcggcgttgacagatgag | | |
| | | gggcaggctcgatttcggccggcgacgtggagctggccagcctcgcaaatcgg | | |
| | | cgaaaacgcctgattttacgcgagtttcccacagatgatgtggacaagcctgggg | | |
| | | ataagtgccctgcggtattgacacttgaggggcgcgactactgacagatgaggg | | |
| | | gcgcgatccttgacacttgaggggcagagtgctgacagatgaggggcgcacct | | |
| | | attgacatttgaggggctgtccacaggcagaaaatccagcatttgcaagggtttcc | | |
| | | gcccgtttttcggccaccgctaacctgtcttttaacctgcttttaaaccaatatttat | | |
| | | aaaccttgtttttaaccagggctgcgccctgtgcgcgtgaccgcgcacgccgaagg | | |
| | | ggggtgcccccccttctcgaaccctcccggcccgctctcgagttggcagcatcac | | |
| | | ccataattgtggtttcaaaatcggctccgtcgatactatgttatacgccaactttgaa | | |
| | | aacaactttgaaaaagctgttttctggtatttaaggttttagaatgcaaggaacagtg | | |
| | | aattggagttcgtcttgttataattagcttcttggggtatctttaaatactgtagaaa | | |
| | | agaggaaggaaataataaatggctaaaatgagaatatcaccggaattgaaaaaactga | | |
| | | tcgaaaaataccgctgcgtaaaagatacggaaggaatgtctcctgctaaggtatat | | |
| | | aagctggtgggagaaaatgaaaacctatatttaaaaatgacggacagccggtata | | |
| | | aagggaccacctatgatgtggaacgggaaaaggacatgatgctatggctggaag | | |
| | | gaaagctgcctgttccaaaggtcctgcactttgaacggcatgatggctggagcaa | | |
| | | tctgctcatgagtgaggccgatggcgtcctttgctcggaagagtatgaagatgaac | | |
| | | aaagccctgaaaagattatcgagctgtatgcggagtgcatcaggctctttcactcc | | |
| | | atcgacatatcggattgtccctatacgaatagcttagacagccgcttagccgaattg | | |
| | | gattacttactgaataacgatctggccgatgtggattgcgaaaactgggaagaag | | |
| | | acactccatttaaagatccgcgcgagctgtatgattttttaaagacggaaaagccc | | |
| | | gaagaggaacttgtcttttcccacggcgacctgggagacagcaacatctttgtgaa | | |
| | | agatggcaaagtaagtggctttattgatcttgggagaagcggcagggcggacaa | | |
| | | gtggtatgacattgccttctgcgtccggtcgatcagggaggatatcggggaagaa | | |
| | | cagtatgtcgagctattttttgacttactggggatcaagcctgattgggagaaaata | | |
| | | aaatattatattttactggatgaattgttttagtacctagatgtggcgcaacgatgcc | | |
| | | ggcgacaagcaggagcgcaccgacttcttccGcatcaagtgttttggctctcaggc | | |
| | | cgaggcccacggcaagtatttgggcaagggGtcgctggtattcgtgcagggca | | |
| | | agattcggaataccaagtacgagaaggacggcCagacggtctacgggaccga | | |
| | | cttcattgccgataaggtggattatctggacaccaAggcaccaggcgggtcaaat | | |
| | | caggaataagggcacattgccccggcgtgagtcggGgcaatcccgcaaggag | | |
| | | ggtgAatgaatcggacgtttgaccggaaggcatacaggcaagaactgatcgac | | |
| | | gcggggttttccgccgaggatgccgaaaccatcgcaagccgcaccgtcatgcgt | | |
| | | gcgccccgcgaaaccttccagtccgtcggctcgatggcccagcaagctacggc | | |
| | | caagatcgagcgcgacagcgtgcaactggctccccctgccctgcccgcgccatc | | |
| | | ggccgccgtggagcgttcgcgtcgtctcgaacaggaggcggcaggtttggcga | | |
| | | agtcgatgaccatcgacacgcgaggaactatgacgaccaagaagcgaaaaacc | | |
| | | gccggcgaggacctggcaaaacaggtcagcgaggccaagcaggccgcgttgc | | |
| | | tgaaacacacgaagcagcagatcaaggaaatgcagctttccttgttcgatattgcg | | |
| | | ccgtggccggacacgatgcgagcgatgccaaacgacacggcccgctctgccct | | |
| | | gttcaccacgcgcaacaagaaaatcccgcgcgaggcgctgcaaaacaaggtca | | |
| | | ttttccacgtcaacaaggacgtgaagatcacctacaccggcgtcgagctgcgggc | | |
| | | cgacgatgacgaactggtgtggcagcaggtgttggagtacgcgaagcgcaccc | | |
| | | ctatcggcgagccgatcaccttcacgttctacgagctttgccaggacctgggctg | | |
| | | gtcgatcaatggccggtattacacgaaggccgaggaatgcctgtcgcgcctaca | | |
| | | ggcgacggcgatgggctccacgtccgaccgcgttgggcacctggaatcggtgt | | |
| | | cgctgctgcaccgcttccgcgtcctggaccgtggcaagaaaacgtcccgttgcca | | |
| | | ggtcctgatcgacgaggaaatcgtcgtgctgtttgctggcgaccactacacgaaa | | |
| | | ttcatatgggagaagtaccgcaagctgtcgccgacggcccgacggatgttcgact | | |
| | | atttcagctcgcaccgggagccgtacccgctcaagctggaaaccttccgcctcat | | |
| | | gtgcggatcggattccacccgcgtgaagaagtggcgcgagcaggtcggcgaa | | |
| | | gcctgcgaagagttgcgaggcagcggcctggtggaacacgcctgggtcaatga | | |
| | | tgacctggtgcattgcaaacgctaGggccttgtggggtcagttccggctgggggt | | |
| | | tcagccagcGctttactgagatctcctgtggttggcatgcacatacaaatggacga | | |
| | | acggataaAccttttcacgcccttttaaatatccgattattctaataaacgctctttt | | |
| | | ctcttAggtttacccgccaatatatcctgtcaaacactgatagtttaaactgaagggg | | |
| | | gAaacgacaatctgctagtggatcccccggccgggctgcaggtcactggatttt | | |
| | | ggTtttaggaattagaaattttattgatagaagtattttacaaatacaaatacatacT | | |
| | | aagggtttcttatatgctcaacacatgagcgaaaccctataagaaccctaattcCct | | |
| | | tatctgggaactactcacacattattctggagaaaaatagagagagatagatTtgt | | |
| | | agagagagactggtgatttttgcggactctaggctatgaccatgattacgcCaag | | |
| | | cttgcatgcctgcagccaggtttcgtcctcacggactcatcaacccggaaaAcac | | |
| | | atccgggtacctgggcccctaccgggggtaacggggggattcgaacccctcGc | | |
| | | tttattacgtgcctgcggatgtatatgaaccatatacatttgacccgcgcgatccaag | | |
| | | acacaacccttcgatttaagtggagggaaaaacactatgcgttatcgtacGcacc | | |
| | | acgtgtgattacggacacaatccgttatttattatgcatcttgactacctCaagttgc | | |
| | | aggaccgcggccgccctagGCCCGGGCTATTATTGAGCAG | | |
| | | AATCAGAATCATCTTCTTCATCAGAAACAACAAC | | |
| | | ATCTTGAGTACCAGAATCAAGAACATTAAGATAA | | |
| | | TTAGTAGGAAGAATATCACCAAGTTCATGAATTT | | |
| | | GATTAATTTGTTGTCTAGCAGCAAGAAGAGCAGC | | |
| | | AAGTTGAGCTTCAGCATAATCAGAAACAGAATCC | | |
| | | TTAGGAACATTCTTAACAATTTCAGCATTAGAAC | | |
| | | CAGGAAAAAAAACAACAATATAATGATCAGAAA | | |
| | | ATTCAAAAACAGGAACAACGTTCTTAATAGGTCT | | |
| | | CTTAGGAAAACCCATAGAATAAAGTCTCTTTTCA | | |
| | | AAAGAACCCATAGGATCCTTTTCAGAAACAGCAA | | |
| | | CAAGATCTTCAGAAGCTTCCATAACCTTAAAATC | | |
| | | AGGAAGAGCGTAAGGTTCAGAAATCTTATAATAA | | |
| | | GTCATACCAATTTGCTTTTGAAGACCCTTAGCTTC | | |
| | | AGCATACTTAGGACAATGTTGTTCAAGACCATAA | | |
| | | AGCTTAGCCTTAGCAGCCTTCTTATTAATAGGAG | | |
| | | GAGCAAGTTGCTTTTGCTTAGGCTTAACAACTTCA | | |
| | | GCCTTTTGCTTAGCAGGCTTCTTAGCTTGATAACC | | |
| | | AGAAGGACCAGGTTGAAGTTGTTGAGGTCTAGCA | | |
| | | GAAGCTCTAGAAAGTTGTTGAGGTTGAGATTGTT | | |
| | | GCTTCTTCTTAGTAGGCTTAGAAATATCTCTAAGA | | |
| | | CCTTCAGGAACAGAAACGTTATGTCTTTCAATAG | | |
| | | CTTGCATAATTTCAGCAATAAGAGCAGAAGAAGG | | |
| | | ATCAGTAAGAGTAAAAGTAACAATAGACATGGC | | |
| | | GCGCCTTAattaatgacaagaacacgaactgagatggagtagtgatactgt | | |
| | | aagatctatttaaaacgaatccgattcggcgacagtagcctccgaatcatcatcgat | | |
| | | taaattattctttttaaaactcattcctccaaaatccttaacatttctatagttcttg | | |
| | | ttcggcactgaccgatcactactactatttttccctttgcggacatcactcttttttc | | |
| | | cggttcgagatcgaaactttgcaagcctgatcgacatagggacatcttccatgaactc | | |
| | | atcaacgacttcttctgtaagttccatgggccctccgtctctcacgtttgtaatcttc | | |
| | | aacctaattttatattatttctataaacaatacacaccgacacaaactccagagaaag | | |
| | | tctctcacggacagaaacccgctgacatcttcacatttctaatattaactaaaacttg | | |
| | | ccagacgtttttcatcgcgtcctgggtggttatagcataattgggaacgaccttgaac | | |
| | | tgaaatcttttctttgcagctgctgtgtagtaagatccgagagtggcctcgtcggctc | | |
| | | tttccatccttttgtccaccagacacacgctcacacctcctctgcaattgtcaggcaa | | |
| | | gttccactcgcccgtgacgaccaaaccggctaaacagacgtatccactatcaataagc | | |
| | | ttaactcctttaagaaggttcacccctgacaatgactcattctcatgaaccattattt | | |
| | | tatcaactttggaacacataacactctttacaggggtaaacatcgacggtaagatctt | | |
| | | ctccatttttgtcaggtcgataaactcattgatattcacttttcctttaacaactaga | | |
| | | gccatctataaacaaacttctaaaaagaactttatcagacaaatacttcaccagactt | | |
| | | ttataaacaaacgaacctggaggggcggtcttatgaacctcccatacagcgtcgtcca | | |
| | | actgtgtgtaatacgcacaattgttcaacgaaacagcaacatcacaaagagaccttct | | |
| | | gaactcctccaagtgttcccaatccttgatgtgtttagcaccaagtttcgagatcaac | | |
| | | tttaggggatcgtaatacacaatgcatcctctgtcgtgatgtattacatatcttccgc | | |
| | | aaaagtatccatactgttttttaaacagttttgcttcaaaattccacataagattcgc | | |
| | | ggagtgttgcacatccggaaactcacaaccctttggaaagtacagcagactatcgtca | | |
| | | ccgcaaaaggctcctttgattattttctccatcggaagcatcgaggccaaacatgca | | |
| | | gcaatgatcacagtgtttccaatgaacgtcgtgacgtccccgctctttctttgatacc | | |
| | | agatgcaagtttttatacctgcggtataatccttgagggtggtctttctatgcccttg | | |
| | | tttccaaacttctcccaagaagtcttcgaaacccaatcttcgccagatctcgtattct | | |
| | | actgcacagtggaattcattctgagatttgtcgtattttgatatatccagctccaaga | | |
| | | catccatcggcacatgactgtcgagatctccgaagaaatcctcaatctgcgctggtgt | | |
| | | ctttcttgtgaaaaacaaaaatctgctcgaatcaacactgtccagtaattgcctagta | | |
| | | agctcactaaacaacgggccgaatattgcattgatcttttttgaatggtacacaatcg | | |
| | | tctgcaaagccgggtactccgtttggattgaagtgtccaacttttgtttgggttgtgc | | |
| | | tttaatcatgtgtctgtactgatcaactgctggcaaatccacaaaatcaaaatctgcg | | |
| | | agctggcctattgttacctgttcctgcttttctaaccatctattgagagactctctac | | |
| | | tgaacaaagaaacatttttatttggttttcttttttctttaagcaaataactatcaaa | | |
| | | aaacttatctacaaccaaagatgcagtattttcaatatcaatgatgccagacaactcg | | |
| | | ggtgcgttaaagtttcttttaatcatcgccactaaattttccaatagtccagtctggc | | |
| | | gtggcatttctgccgccgttcgtaccataggtattagtggtttgatttgatccttagg | | |
| | | cgcagcaacagacttagacatatccaatatgcaatctttgacattcaatgaaatgtca | | |
| | | gtcaacctcatggtaacagcatcaaaattattcatcatggtgctgttgcctgggagac | | |
| | | acttatcatagtaaaactgcatatcagaaatatcaccagtctttggcgctgcaacaaa | | |
| | | aagattggaacctttgaacaccgagtcaatctgtaattgctattgtgttcctgcatcg | | |
| | | accttatacatatctaacaagtacgagctaagtttctctagatctctaatgatactaa | | |
| | | ctaaaggatccataacaacagtgtagtacttgagcgaacaggtgtgccttgacaatgc | | |
| | | gaccaaaacatgtgggctgtctcctgcaatgatggagaccggtgtaggggttaaccta | | |
| | | actagtgaaacatcagagtatgtctcgccttgcacttcatgcacagtgtgaacatctg | | |
| | | aataccctcttgaaagcagagcttctttatccgattgggtaaaagtcaagatcttgcc | | |
| | | atgcaagggttttgagatcggattgatcacggcggctccgccgaccatctcctgcgaa | | |
| | | acagactttttaaccgaagaagtgctcatgacaaagccctcatatctcctgttcagat | | |
| | | aatgtgtgacatcggctggacaacggagagtagttctgcgtgtctccacctcgtca | | |
| | | acttccaatttggcaaaatgggcggggtacgggaatcctgaaactctattgatgtat | | |
| | | ggaatctgctgtgtgtctccgtaaacatatgcaatttcgcacaatgacatcgccaca | | |
| | | agaaaattaacacaaccagtatgcaacatcaacccttcatcaatgaataacctcttg | | |
| | | aactgacagcgtgtgcttttcccaaaattcatcatgaaagaatcaacggttttaacgt | | |
| | | tgtccttcgtggccacaataatccctgaggaattcgcacgtcttctgatcatttccgc | | |
| | | ggcttgcttcccaggtactaaaattagatcttcatcaaaattaaccctggaaagaatt | | |
| | | tctttggtttttccacagcccggaactccgtccacaagaacaacctttgcgctactg | | |
| | | acatgcggttctccgtttcgaagcagtctgcgcagagttctgagtttcgccatgtcg | | |
| | | gaataaacaacagactcagagctaacagctactcttctccaatcatcgcatgtcac | | |
| | | cacaccctgctcatcatattccaaaagcgccacatgatacttcctcgcgtgggtttc | | |
| | | aacaacaccccatgcatgactcttggccgttggtttgattaaccacttcctagatgc | | |
| | | aacatccaagactccaaacttttgacgggtttcaaggtcaatagcagctgtatctttg | | |
| | | aggatcttgacgagattcgacaccgcagcagatagtgatgctaccaggctatcga | | |
| | | taaagtttttcatttgctgaactttaatcggacccgtgtacacaatcgagctcatctg | | |
| | | cttacgaattaacgaatctgccgtcgccatatgaaactgctctaaagactctatctcc | | |
| | | tcgttcttagaataggacgattccggatgatctccagcaagaccagctaattgtaact | | |
| | | ctcctctggccatcgaacccttcatggacggttcttcaacttctcttgaggtaactac | | |
| | | caatgcaccttctgaagccttctcttgatcctgtaaagctagcgcaacattcgcctca | | |
| | | gtaggtcgttcaaatgtgagagtcagaccgctctcattgctcatgaccgcgactata | | |
| | | accttcgctgccgtcattgggtcaacttccaaagattggcacatctgggaaaaaac | | |
| | | atcaacatcgaatttgtcagactcccttaacaccgataattctgaaagtgcattgtac | | |
| | | atcacttccgtttcttccatcttcttcctaatgtcaagcgcaggcatgtccacagagg | | |
| | | ccttgtactcagtcactaatctgtcgtggaaggtcacatatagatcaggcaccctga | | |
| | | tctctaatgcgtcgcctgccactctgataagtttcctgttcaagagcctctctttcac | | |
| | | ggagggaaatgcgttcccaaacgccagcgaaatctcatcccacacatgctggcac | | |
| | | accgttttcgaaccgagactaaacttgctaatcagtaagtcatcctttagaacggca | | |
| | | agcttagtatgcaggtaaaacgtcatggacaaggattgtaacaaagatttgtccac | | |
| | | atcccattcggacctcgctgtcacaccgttaatgattaccctcgatcgaatcgattc | | |
| | | gacgaaggacaaaacatttgcgtatgtaagagctttcgcctggtatgttcgaatgtg | | |
| | | gttaagcactgtaaacacgaaatccttggacactaagacttccttgcgcgtcctctt | | |
| | | actagtctccaaagaaatgtcgaataatggtacgatgaccatatccctcattttggg | | |
| | | aaaccagtaattgactgatgatgaatcctcaaggaggattctctcgctgttgcacatt | | |
| | | gcaagagtctttttgtaatgccatgcgtcttccattgcagtataaaactgctcactat | | |
| | | ctacacttttatgggccacacctttgtacaaaagaaaagtatctattctagaaaactt | | |
| | | acaaaaccaggtattaactctggtgactaaaaactccttcatgtaaacctctctatta | | |
| | | gaggccgggaagtaagttttgcacacatacttaagaatattagaataactatgacagt | | |
| | | aattaagagtactctctgatgcaaaagaaaaggtcaacttgtctccatcgcgcgaaa | | |
| | | aacacgcgttgatttcgtccaaattgacgcatgaatcttcaagaagcaggttctcgg | | |
| | | agaagtggaaagcggcatagcacgtatggacatttttcctcaagagtgccgcccc | | |
| | | gaactcatcggctggtatgtcatatatgctgtgtagcgcaatggcatacactctgcc | | |
| | | tgattgctgcatcggctgatgttcgcatgtctggaaagtattgtgacagacagcgtc | | |
| | | ttcaggaatttctgcgtatctgtcaaatgcttccttttggaagttggggactgttttc | | |
| | | ccccctctctctagcctagaaaggtatagttcaatactgtctttctggccttcgtgcc | | |
| | | gcatgatgtctcgaacgtccaggttgggcatgcagcagtgtacatatgctcgtccctt | | |
| | | gaacagatgcgatgcaaaattcccgcctatgtcataagtcaatgatccgtagggaat | | |
| | | ttgcatcatcagatattccagttctaaagatcgcaatccacctgcaagcgaatgcac | | |
| | | ggcattttgcgtgttataaaatgtaatttggaattctggatacgcccgggtagcaata | | |
| | | agcgtctgctcctcgcttattacttttgaaaagttcaccttgggcctgcggtcacgag | | |
| | | cgttaaactcttcaaccgctgtgtcgtaaagacgacgctttgctagatcattgacca | | |
| | | aggagttgtttcctcggacagtgtccagcaaagctgatgtggtagctgtctgtgtgt | | |
| | | atgccattgtaattgtaaaTagtaattgtaatgttgtctgttgtttgttgttgttggt | | |
| | | aattgttgtaaaaataC | | |
| SEQ ID | RB T- | tgacaggatatattggcgggtaaac | DNA | Syn* |
| NO: 32 | DNA | | | |
| | repeat | | | |
| SEQ ID | Comp- | gtttacccgccaatatatcctgtca | DNA | Syn* |
| NO: 33 | limentary | | | |
| | strand of | | | |
| | RB T- | | | |
| | DNA | | | |
| | repeat | | | |
| SEQ ID | trbo fwd | gatgattcggaggctactgtc | DNA | Syn* |
| NO: 34 | | | | |
| SEQ ID | p29 rev | CAGAAGGACCAGGTTGAAGTTG | DNA | Syn* |
| NO: 35 | | | | |
| SEQ ID | pTRBOrev | gccaggacgttgaactccatc | DNA | Syn* |
| NO: 36 | | | | |
| SEQ ID | pBluescrip | TCGAGGTCGACGGTATC | DNA | Syn* |
| NO: 37 | tKS | | | |

### EQUIVALENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims. All references are herein incorporated in their entirety for any and all purposes.

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. Certain references are identified by an Arabic numeral, the complete bibliographic citation for each of which immediately precedes the claims. In case of conflict, the present specification, including definitions, will control.

Other aspects are set forth within the following claims.

### REFERENCES

[1] R. Twarock, P. G. Stockley, RNA-Mediated Virus Assembly: Mechanisms and Consequences for Viral Evolution and Therapy, Annu Rev Biophys 48 (2019) 495-514.
[2] N. F. Steinmetz, S. Lim, F. Sainsbury, Protein cages and virus-like particles: from fundamental insight to biomimetic therapeutics, Biomater Sci 8 (10) (2020) 2771-2777.
[3] P. L. Chariou, A. B. Dogan, A. G. Welsh, G. M. Saidel, H. Baskaran, N. F. Steinmetz, Soil mobility of synthetic and virus-based model nanopesticides, Nat Nanotechnol 14 (7) (2019) 712-718.
[4] J. Cao, R. H. Guenther, T. L. Sit, S. A. Lommel, C. H. Opperman, J. A. Willoughby, Development of abamectin loaded plant virus nanoparticles for efficacious plant parasitic nematode control, ACS Appl Mater Interfaces 7 (18) (2015) 9546-53.
[5] P. J. Hoopes, R. J. Wagner, K. Duval, K. Kang, D. J. Gladstone, K. L. Moodie, M. Crary-Burney, H. Ariaspulido, F. A. Veliz, N. F. Steinmetz, S. N. Fiering, Treatment of Canine Oral Melanoma with Nanotechnology-Based Immunotherapy and Radiation, Mol Pharm 15 (9) (2018) 3717-3722.
[6] Y. H. Chung, D. Church, E. C. Koellhoffer, E. Osota, S. Shukla, E. P. Rybicki, J. K. Pokorski, N. F. Steinmetz, Integrating plant molecular farming and materials research for next-generation vaccines, Nat Rev Mater 7 (5) (2022) 372-388.
[7] J. M. Royal, C. A. Simpson, A. A. McCormick, A. Phillips, S. Hume, J. Morton, J. Shepherd, Y. Oh, K. Swope, J. L. DeBeauchamp, R. J. Webby, R. W. Cross, V. Borisevich, T. W. Geisbert, J. K. Demarco, B. Bratcher, H. Haydon, G. P. Pogue, Development of a SARS-CoV-2 Vaccine Candidate Using Plant-Based Manufacturing and a Tobacco Mosaic Virus-like Nano-Particle, Vaccines (Basel) 9 (11) (2021).
[8] P. H. Lizotte, A. M. Wen, M. R. Sheen, J. Fields, P. Rojanasopondist, N. F. Steinmetz, S. Fiering, In situ vaccination with cowpea mosaic virus nanoparticles suppresses metastatic cancer, Nat Nanotechnol 11 (3) (2016) 295-303.
[9] M. Brasch, R. M. Putri, M. V. de Ruiter, D. Luque, M. S. Koay, J. R. Caston, J. J. Cornelissen, Assembling Enzymatic Cascade Pathways inside Virus-Based Nanocages Using Dual-Tasking Nucleic Acid Tags, J Am Chem Soc 139 (4) (2017) 1512-1519.
[10] M. Nuzzaci, G. Piazzolla, A. Vitti, M. Lapelosa, C. Tortorella, I. Stella, A. Natilla, S. Antonaci, P. Piazzolla, Cucumber mosaic virus as a presentation system for a double hepatitis C virus-derived epitope, Arch Virol 152 (5) (2007) 915-28.
[11] Y. Ren, S. M. Wong, L. Y. Lim, Folic acid-conjugated protein cages of a plant virus: a novel delivery platform for doxorubicin, Bioconjug Chem 18 (3) (2007) 836-43.
[12] R. Toy, P. M. Peiris, K. B. Ghaghada, E. Karathanasis, Shaping cancer nanomedicine: the effect of particle shape on the in vivo journey of nanoparticles, Nanomedicine (Lond) 9 (1) (2014) 121-34.
[13] E. C. Locali-Fabris, J. Freitas-Astua, A. A. Souza, M. A. Takita, G. Astua-Monge, R. Antonioli-Luizon, V. Rodrigues, M. Targon, M. A. Machado, Complete nucleotide sequence, genomic organization and phylogenetic analysis of Citrus leprosis virus cytoplasmic type, J Gen Virol 87 (Pt 9) (2006) 2721-2729.
[14] J. C. V. Rodrigues, Relac, õespatógeno-vetor-planta no sistema leprose dos citros, Centro de Energia Nuclear na Agricultura, Universidade de Sa o Paulo, 2000.
[15] J. Freitas-Astua, P. L. Ramos-Gonzalez, G. D. Arena, A. D. Tassi, E. W. Kitajima, Brevipalpus-transmitted viruses: parallelism beyond a common vector or convergent evolution of distantly related pathogens?, Curr Opin Virol 33 (2018) 66-73.
[16] C. Chabi-Jesus, P. L. Ramos-Gonzalez, M. Postclam-Barro, R. S. Fontenele, R. Harakava, R. B. Bassanezi, A. S. Moreira, E. W. Kitajima, A. Varsani, J. Freitas-Astua, Molecular Epidemiology of Citrus Leprosis Virus C: A New Viral Lineage and Phylodynamic of the Main Viral Subpopulations in the Americas, Front Microbiol 12 (2021) 641252.
[17] R. C. Pascon, J. P. Kitajima, M. C. Breton, L. Assumpcao, C. Greggio, A. S. Zanca, V. K. Okura, M. C. Alegria, M. E. Camargo, G. G. Silva, J. C. Cardozo, M. A. Vallim, S. F. Franco, V. H. Silva, H. Jordao, Jr., F. Oliveira, P. F. Giachetto, F. Ferrari, C. I. Aguilar-Vildoso, F. J. Franchiscini, J. M. Silva, P. Arruda, J. A. Ferro, F. Reinach, A. C. da Silva, The complete nucleotide sequence and genomic organization of Citrus Leprosis associated Virus, Cytoplasmatic type (CiLV-C), Virus Genes 32 (3) (2006) 289-98.
[18] M. O. Leastro, E. W. Kitajima, M. S. Silva, R. O. Resende, J. Freitas-Astua, Dissecting the Subcellular Localization, Intracellular Trafficking, Interactions, Membrane Association, and Topology of Citrus Leprosis Virus C Proteins, Front Plant Sci 9 (2018) 1299.
[19] D. B. Kuchibhatla, W. A. Sherman, B. Y. Chung, S. Cook, G. Schneider, B. Eisenhaber, D. G. Karlin, Powerful sequence similarity search methods and in-depth manual analyses can identify remote homologs in many apparently “orphan” viral proteins, J Virol 88 (1) (2014) 10-20.
[20] A. Zeltins, Construction and characterization of virus-like particles: a review, Mol Biotechnol 53 (1) (2013) 92-107.
[21] J. A. Lindbo, TRBO: a high-efficiency tobacco mosaic virus RNA-based overexpression vector, Plant Physiol 145 (4) (2007) 1232-40.
[22] M. A. Plesha, T. K. Huang, A. M. Dandekar, B. W. Falk, K. A. McDonald, Optimization of the bioprocessing conditions for scale-up of transient production of a heterologous protein in plants using a chemically inducible viral amplicon expression system, Biotechnol Prog 25 (3) (2009) 722-34.
[23] M. O. Mohsen, A. C. Gomes, M. Vogel, M. F. Bachmann, Interaction of Viral Capsid-Derived Virus-Like Particles (VLPs) with the Innate Immune System, Vaccines (Basel) 6 (3) (2018).
[24] E. W. Kitajima, G. W. Muller, A. S. Costa, W. Yuki, Short, rod-like particles associated with Citrus leprosis, Virology 50 (1) (1972) 254-8.
[25] V. Yusibov, A. Kumar, A. North, J. E. Johnson, L. S. Loesch-Fries, Purification, characterization, assembly and crystallization of assembled alfalfa mosaic virus coat protein expressed in Escherichia coli, J Gen Virol 77 (Pt 4) (1996) 567-73.
[26] J. Choi, L. S. Loesch-Fries, Effect of C-terminal mutations of alfalfa mosaic virus coat protein on dimer formation and assembly in vitro, Virology 260 (1) (1999) 182-9.
[27] K. Fukuyama, S. S. Abdel-Meguid, J. E. Johnson, M. G. Rossmann, Structure of a T=1 aggregate of alfalfa mosaic virus coat protein seen at 4.5 A resolution, J Mol Biol 167 (4) (1983) 873-90.
[28] R. Hull, Studies on alfalfa mosaic virus. 3. Reversible dissociation and reconstitution studies, Virology 40 (1) (1970) 34-47.
[29] R. A. Driedonks, P. C. Krijgsman, J. E. Mellema, Alfalfa mosaic virus protein polymerization, J Mol Biol 113 (1) (1977) 123-40.
[30] J. F. Bol, B. Kraal, F. T. Brederode, Limited proteolysis of alfalfa mosaic virus: influence on the structural and biological function of the coat protein, Virology 58 (1) (1974) 101-10.
[31] A. Kumar, V. S. Reddy, V. Yusibov, P. R. Chipman, Y. Hata, I. Fita, K. Fukuyama, M. G. Rossmann, L. S. Loesch-Fries, T. S. Baker, J. E. Johnson, The structure of alfalfa mosaic virus capsid protein assembled as a T=1 icosahedral particle at 4.0-A resolution, J Virol 71 (10) (1997) 7911-6.

1. A nanoparticle comprising a cytoplasmic type citrus leprosis virus-like particle (CiLV-C) p29 protein coat and an optional agent encapsulated with the p29 protein coat.

2. The nanoparticle of claim 1, wherein the p29 protein comprises SEQ ID NO: 2.

3. The nanoparticle of claim 1, with the proviso that the CiLV-C virus like-particle lacks the p61 glycoprotein and the integral membrane protein p24.

4. The nanoparticle of claim 1, wherein the CiLV-C virus-like particle further comprises the CiLV-C movement protein (MP) p32, optionally the MP of SEQ ID NO: 6, or a tobacco mosaic virus (TMV) movement protein (MP), optionally the TMV MP of SEQ ID NO: 6.

5. The nanoparticle of claim 1, wherein the optional agent comprises a therapeutic agent, that further optionally comprises a polynucleotide, optionally an RNA or a DNA molecule.

6. The nanoparticle of claim 1, wherein the optional agent is a therapeutic agent, that is optionally antisense RNA.

7. The nanoparticle of claim 1, wherein the optional agent comprises an agricultural agent that is further optionally a pesticide.

8. A population of any of the nanoparticles of claim 1, that are optionally detectably labeled.

9. The population of claim 8, wherein the nanoparticles are the same or different from each other.

10. A method to deliver a CiLV-C nanoparticle to a cell comprising contacting the cell with the CiLV-C virus-like nanoparticle of claim 1.

11. The method of claim 10, wherein the contacting is in vitro or in vivo.

12. A method to deliver a CiLV-C nanoparticle to a subject in need thereof, comprising administering the CiL V-C virus-like nanoparticle of claim 1.

13. A polynucleotide encoding the nanoparticle of claim 1, optionally wherein the polynucleotide is RNA or DNA.

14. A recombinant polynucleotide comprising: an expression vector; a TMV replicase; a polynucleotide encoding a CiLV-C p29 protein, a movement protein p32 or a TMV movement protein.

15. A recombinant polynucleotide of claim 14, comprising the polynucleotide shown in FIG. 7B (SEQ ID NO: 7).

16. An organism or host cell comprising the recombinant polynucleotide of claim 13, optionally wherein the organism comprises Brevipalpus spp. or Agrobacterium tumefaciens or wherein the host cell comprises N. benthamiana.

17-18. (canceled)

19. A CiLV-C virus particle produced by expressing the polynucleotide of claim 13 in an organism infected plant cell.

20. The CiLV-C virus particle of claim 19, wherein the organism comprises A. tumefaciens and the plant cell comprises N. benthamiana.

21. A method to package an agent comprising contacting the CiLV-C virus nanoparticle of claim 1 with the agent.

22. The method of claim 21, further comprising isolating the CiLV-C virus particle from the plant cell.
